SEQUENCE LISTING (9 sequences)

SEQ ID NO: 1 - protein, 210 aa, Homo sapiens
MADGSSDAAREPRPAPAPIRRRSSNYRAYATEPHAKKKSKISASRKLQLKTLLLQIAKQELEREAEERRGEKGRALSTRCQPLELAGLGFAELQDLCRQLHARVDKVDEERYDIEAKVTKNITEIADLTQKIFDLRGKFKRPTLRRVRISADAMMQALLGARAKESLDLRAHLKQVKKEDTEKENREVGDWRKNIDALSGMEGRKKKFES

SEQ ID NO: 2 - protein, 531 aa, Homo sapiens
MSKPHSEAGTAFIQTQQLHAAMADTFLEHMCRLDIDSPPITARNTGIICTIGPASRSVETLKEMIKSGMNVARLNFSHGTHEYHAETIKNVRTATESFASDPILYRPVAVALDTKGPEIRTGLIKGSGTAEVELKKGATLKITLDNAYMEKCDENILWLDYKNICKVVEVGSKIYVDDGLISLQVKQKGADFLVTEVENGGSLGSKKGVNLPGAAVDLPAVSEKDIQDLKFGVEQDVDMVFASFIRKASDVHEVRKVLGEKGKNIKIISKIENHEGVRRFDEILEASDGIMVARGDLGIEIPAEKVFLAQKMMIGRCNRAGKPVICATQMLESMIKKPRPTRAEGSDVANAVLDGADCIMLSGETAKGDYPLEAVRMQHLIAREAEAAMFHRKLFEELVRASSHSTDLMEAMAMGSVEASYKCLAAALIVLTESGRSAHQVARYRPRAPIIAVTRNPQTARQAHLYRGIFPVLCKDPVQEAWAEDVDLRVNFAMNVGKARGFFKKGDVVIVLTGWRPGSGFTNTMRVVPVP

SEQ ID NO: 3 - protein, 531 aa, Homo sapiens
MSKPHSEAGTAFIQTQQLHAAMADTFLEHMCRLDIDSPPITARNTGIICTIGPASRSVETLKEMIKSGMNVARLNFSHGTHEYHAETIKNVRTATESFASDPILYRPVAVALDTKGPEIRTGLIKGSGTAEVELKKGATLKITLDNAYMEKCDENILWLDYKNICKVVEVGSKIYVDDGLISLQVKQKGADFLVTEVENGGSLGSKKGVNLPGAAVDLPAVSEKDIQDLKFGVEQDVDMVFASFIRKASDVHEVRKVLGEKGKNIKIISKIENHEGVRRFDEILEASDGIMVARGDLGIEIPAEKVFLAQKMMIGRCNRAGKPVICATQMLESMIKKPRPTRAEGSDVANAVLDGADCIMLSGETAKGDYPLEAVRMQHLIAREAEAAIYHLQLFEELRRLAPITSDPTEATAVGAVEASFKCCSGAIIVLTKSGRSAHQVARYRPRAPIIAVTRNPQTARQAHLYRGIFPVLCKDPVQEAWAEDVDLRVNFAMNVGKARGFFKKGDVVIVLTGWRPGSGFTNTMRVVPVP

SEQ ID NO: 4 - protein, 134 aa, Homo sapiens
MDPQTAPSRALLLLLFLHLAFLGGRSHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 5 - protein, 201 aa, Homo sapiens
MKWVWALLLLAALGSGRAERDCRVSSFRVKENFDKARFSGTWYAMAKKDPEGLFLQDNIVAEFSVDETGQMSATAKGRVRLLNNWDVCADMVGTFTDTEDPAKFKMKYWGVASFLQKGNDDHWIVDTDYDTYAVQYSCRLLNLDGTCADSYSFVFSRDPNGLPPEAQKIVRQRQEELCLARQYRLIVHNGYCDGRSERNLL

SEQ ID NO: 6 - protein, 359 aa, Homo sapiens
MKATIILLLLAQVSWAGPFQQRGLFDFMLEDEASGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 7 - protein, 220 aa, Homo sapiens
MGAAARTLRLALGLLLLATLLRPADACSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGCECKITRCPMIPCYISSPDECLWMDWVTEKNINGHQAKFFACIKRSDGSCAWYRGAAPPKQEFLDIEDP

SEQ ID NO: 8 - protein, 446 aa, Homo sapiens
MLLPQLCWLPLLAGLLPPVPAQKFSALTFLRVDQDKDKDCSLDCAGSPQKPLCASDGRTFLSRCEFQRAKCKDPQLEIAYRGNCKDVSRCVAERKYTQEQARKEFQQVFIPECNDDGTYSQVQCHSYTGYCWCVTPNGRPISGTAVAHKTPRCPGSVNEKLPQREGTGKTDDAAAPALETQPQGDEEDIASRYPTLWTEQVKSRQNKTNKNSVSSCDQEHQSALEEAKQPKNDNVVIPECAHGGLYKPVQCHPSTGYCWCVLVDTGRPIPGTSTRYEQPKCDNTARAHPAKARDLYKGRQLQGCPGAKKHEFLTSVLDALSTDMVHAASDPSSSSGRLSEPDPSHTLEERVVHWYFKLLDKNSSGDIGKKEIKPFKRFLRKKSKPKKCVKKFVEYCDVNNDKSISVQELMGCLGVAKEDGKADTKKRHTPRGHAESTSNRQPRKQG

SEQ ID NO: 9 - protein, 543 aa, Homo sapiens
MSSFSYEPYYSTSYKRRYVETPRVHISSVRSGYSTARSAYSSYSAPVSSSLSVRRSYSSSSGSLMPSLENLDLSQVAAISNDLKSIRTQEKAQLQDLNDRFASFIERVHELEQQNKVLEAELLVLRQKHSEPSRFRALYEQEIRDLRLAAEDATNEKQALQGEREGLEETLRNLQARYEEEVLSREDAEGRLMEARKGADEAALARAELEKRIDSLMDEISFLKKVHEEEIAELQAQIQYAQISVEMDVTKPDLSAALKDIRAQYEKLAAKNMQNAEEWFKSRFTVLTESAAKNTDAVRAAKDEVSESRRLLKAKTLEIEACRGMNEALEKQLQELEDKQNADISAMQDTINKLENELRTTKSEMARYLKEYQDLLNVKMALDIEIAAYRKLLEGEETRLSFTSVGSITSGYSQSSQVFGRSAYGGLQTSSYLMSTRSFPSYYTSHVQEEQIEVEETIEAAKAEEAKDEPPSEGEAEEEEKDKEEAEEEEAAEEEEAAKEESEEAKEEEEGGEGEEGEETKEAEEEEKKVEGAGEEQAAKKKD

(19) United States
(12) Patent Application Publication
(10) Pub. No.: US 2025/0027943 A1
(43) Pub. Date: Jan. 23, 2025

(54) BIOMARKER COMPOSITIONS AND METHODS OF USE THEREOF

(71) Applicant: Siemens Healthcare Diagnostics Inc., Tarrytown, NY (US)

(72) Inventors: Arejas James Uzgiris, Berkeley, CA (US); Chris Green, Suisun City, CA (US); Mark Baumeister, Concord, CA (US)

(73) Assignee: Siemens Healthcare Diagnostics Inc., Tarrytown, NY (US)

(21) Appl. No.: 18/684,221

(22) PCT Filed: Sep. 7, 2022
(86) PCT No.: PCT/US2022/076076

§ 371 Date: Feb. 16, 2024

Related U.S. Application Data
(60) Provisional application No. 63/241,518, filed on Sep. 7, 2021.

(51) Int. Cl. G01N 33/573; G01N 33/68

(52) U.S. Cl. CPC G01N 33/573; G01N 33/6893; G01N 2333/9121; G01N 2800/10

(57) ABSTRACT

The present disclosure provides methods and kits for identifying and treating individuals at risk of or suffering from amyloid transthyretin cardiomyopathy. In general, detection or measurement of one or more biomarkers, such as TnI, PKM1, PKM2, NT-proBNP, RBP4, DCN, TIMP2, SMOC-2, NfL, or combinations thereof, assists in the identification of amyloid transthyretin cardiomyopathy. The present disclosure also provides methods for selecting patients for treatment of amyloid transthyretin cardiomyopathy, such as with transthyretin stabilizing agents.

### CROSS REFERENCE TO RELATED APPLICATION

This application claims priority or the benefit under 35 U.S.C. § 119 of U.S. Provisional Application No. 63/241,518, filed on Sep. 7, 2021, the entire contents of which is incorporated herein by reference.

### CROSS-REFERENCE TO A SEQUENCE LISTING

This application contains a Sequence Listing in computer readable form entitled “AttrSEQLST.xml”, created on Sep. 7, 2022, which is 12 KB in size. The computer readable form is incorporated herein by reference.

### TECHNICAL FIELD

The present disclosure relates generally to the fields of molecular biology and cardiac health.

### BACKGROUND

Amyloid transthyretin cardiomyopathy (TTR-CM) is a rare condition that arises due to misfolding of the protein transthyretin (TTR), resulting in amyloid fibril deposition in the heart tissue and ultimately leading to heart failure. Early treatment of TTR-CM is therefore important for improved prognostic impact in affected individuals. However, treatment may be problematically delayed as screening for wild-type transthyretin amyloidosis (ATTRwt) involves performing multiple costly and invasive procedures that are not particularly specific to TTR-CM (including TTR-CM resulting from wild-type ATTR amyloidosis). Moreover, there is a lack of non-invasive, in-vitro screening tests for detecting and/or identifying amyloid transthyretin cardiomyopathy (TTR-CM) resulting from wild-type ATTR amyloidosis (ATTRwt).

Difficulty in identifying TTR-CM resulting from ATTRwt by current methods may contribute to this disease being largely underdiagnosed. Accordingly, there is a need in the art for compositions, kits, and methods for detecting TTR-CM, and particularly TTR-CM resulting from wild-type ATTR amyloidosis, with an acceptable level of specificity. Moreover, there is a continuous need for compositions, assays, devices, and methods for testing, identifying, or staging individuals or groups of individuals having misfolded protein TTR.

### SUMMARY

The present disclosure provides compositions, kits, and methods for detecting and/or identifying TTR-CM, and particularly TTR-CM resulting from wild-type ATTR amyloidosis. Further, the present disclosure provides methods of classifying TTR-CM patients (including TTR-CM resulting from wild-type ATTR amyloidosis) compared to normal individuals or individuals experiencing other heart conditions. Additionally, the present disclosure recognizes for the first time that certain biomarkers (e.g., ATTR Biomarkers, e.g., troponin I (TnI), pyruvate kinase muscle isoform 1 (PKM1), pyruvate kinase muscle isoform 2 (PKM2), N-terminal-pro hormone B-type natriuretic peptide (NT-proBNP), retinol binding protein 4 (RBP4), decorin (DCN), tissue inhibitor of metalloproteinase 2 (TIMP2), SPARC related modular calcium binding 2 (SMOC-2), neurofilament light chain (NfL), and combinations thereof) can assist in detecting and/or diagnosing TTR-CM, and/or assist in classification of TTR-CM patients. The present disclosure further recognizes that these biomarkers (e.g., ATTR Biomarkers, e.g., TnI, PKM1, PKM2, NT-proBNP, RBP4, DCN, TIMP2, SMOC-2, NfL, or combinations thereof) in compositions, kits, and methods can be useful for classifying, detecting and/or diagnosing TTR-CM (including TTR-CM resulting from wild-type ATTR amyloidosis) without the need for performing invasive or costly tests. This represents a significant advancement in patient care, as TTR-CM resulting from ATTRwt can be detected and identified by methods that are more comfortable for the patient, inflict less harm to the patient, and/or decrease the amount of time a patient needs to recover following the detection and/or diagnostic method.

In addition, the present disclosure provides that certain biomarkers (e.g., ATTR Biomarkers, e.g., TnI, PKM1, PKM2, NT-proBNP, RBP4, DCN, TIMP2, SMOC-2, NfL, or combinations thereof) are useful in detecting TTR-CM with improved sensitivity. The increased sensitivity achieved with ATTR Biomarkers described herein decreases the number of false negatives obtained when detecting and/or identifying TTR-CM. The decrease in false negatives will, in turn, help to ensure that more TTR-CM patients receive earlier treatments critical for reducing signs, symptoms, and conditions associated with TTR-CM, as well as promoting long-term survival of TTR-CM patients.

Among other things, the present disclosure provides a method including detecting a level of each of two or more transthyretin amyloidosis (ATTR) Biomarkers in a sample. In some embodiments, a sample is obtained from a subject.

The present disclosure also provides a method including: (a) detecting a level of each of two or more ATTR Biomarkers in a sample obtained from a subject to obtain an ATTR Biomarker profile, and (b) using the ATTR Biomarker profile to compute a ATTR Biomarker score.

In some embodiments of methods provided herein, two or more ATTR Biomarkers include (i) troponin I (TnI), (ii) pyruvate kinase muscle isoform 1 (PKM1), (iii) pyruvate kinase muscle isoform 2 (PKM2), (iv) N-terminal-pro hormone B-type natriuretic peptide (NT-proBNP), (v) retinol binding protein 4 (RBP4), (vi) tissue inhibitor of metalloproteinase 2 (TIMP2), (vii) neurofilament light chain (NfL), or (viii) a combination thereof.

In some embodiments, two or more ATTR Biomarkers include TnI and PKM1. In some embodiments, two or more ATTR Biomarkers include TnI and PKM2. In some embodiments, two or more ATTR Biomarkers include TnI, PKM1, and PKM2.

In some embodiments, two or more ATTR Biomarkers include TnI, PKM2, NT-proBNP, and RBP4. In some embodiments, two or more ATTR Biomarkers include TnI, PKM2, and NT-proBNP. In some embodiments, two or more ATTR Biomarkers include TnI, PKM2, and RBP4. In some embodiments, two or more ATTR Biomarkers include TnI, PKM1, and NT-proBNP. In some embodiments, two or more ATTR Biomarkers include TnI, PKM1, and RBP4. In some embodiments, two or more ATTR Biomarkers include TnI, PKM1, PKM2, NT-proBNP, and RBP4. In some embodiments, two or more ATTR Biomarkers include TnI, PKM1, PKM2, and NT-proBNP. In some embodiments, two or more ATTR Biomarkers include TnI, PKM1, PKM2, and RBP4. In some embodiments, two or more ATTR Biomarkers include NT-proBNP, RBP4, and TnI. In some embodiments, two or more ATTR Biomarkers include RBP4, SMOC-2, and TnI. In some embodiments, two or more ATTR Biomarkers include DCN, NT-proBNP, and TnI. In some embodiments, two or more ATTR Biomarkers include DCN, RBP4, and TnI. In some embodiments, two or more ATTR Biomarkers include NT-proBNP, SMOC-2, and TnI. In some embodiments, two or more ATTR Biomarkers include NT-proBNP, TIMP2, and TnI. In some embodiments, two or more ATTR Biomarkers include NT-proBNP and TnI. In some embodiments, two or more ATTR Biomarkers include DCN and TnI. In some embodiments, two or more ATTR Biomarkers include DCN, TIMP2, and TnI. In some embodiments, two or more ATTR Biomarkers include RBP4 and TnI. In some embodiments, two or more ATTR Biomarkers include RBP4, TIMP2, and TnI. In some embodiments, two or more ATTR Biomarkers include DCN, SMOC-2, and TnI. In some embodiments, two or more ATTR Biomarkers include TIMP2 and TnI. In some embodiments, two or more ATTR Biomarkers include SMOC-2, TIMP2, and TnI.

In some embodiments, two or more ATTR Biomarkers do not include PKM1. In some embodiments, two or more ATTR Biomarkers do not include PKM2. In some embodiments, two or more ATTR Biomarkers do not include either PKM1 or PKM2.

In some embodiments, two or more ATTR Biomarkers do not include SMOC-2. In some embodiments, two or more ATTR Biomarkers do not include DCN. In some embodiments, two or more ATTR Biomarkers do not include either SMOC-2 or DCN.

In some embodiments, the step of using an ATTR Biomarker profile to compute an ATTR Biomarker score includes applying an algorithm to the ATTR Biomarker profile to compute an ATTR Biomarker score. In some embodiments, an algorithm is or is derived from a decision tree methodology, a neural boosted methodology, a bootstrap forest methodology, a boosted tree methodology, a K nearest neighbors methodology, a generalized regression forward selection methodology, a generalized regression pruned forward selection methodology, a fit stepwise methodology, a generalized regression lasso methodology, a generalized regression elastic net methodology, a generalized regression ridge methodology, a nominal logistic methodology, a support vector machines methodology, a discriminant methodology, a naïve Bayes methodology, or a combination thereof. In some embodiments, an algorithm is or is derived from a decision tree methodology, a neural boosted methodology, a bootstrap forest methodology, a boosted tree methodology, a generalized regression lasso methodology, a generalized regression elastic net methodology, a generalized regression ridge methodology, a nominal logistic methodology, a support vector machines methodology, a discriminant methodology, or a combination thereof. In some embodiments, an algorithm is or is derived from a decision tree methodology, a neural boosted methodology, a bootstrap forest methodology, a boosted tree methodology, a support vector machines methodology, or a combination thereof.

In some embodiments, a method described herein includes the step of using the ATTR Biomarker score to determine if a subject from which the sample was obtained is at risk of or suffering from transthyretin amyloid cardiomyopathy (TTR-CM). In some embodiments, a method described herein includes the step of using the ATTR Biomarker score to diagnose a subject with TTR-CM. In some embodiments, a method described herein includes the step of using the ATTR Biomarker score to determine if a subject from which the sample was obtained is selected for one or more cardiomyopathy tests. In some embodiments, a method described herein includes the step of using the ATTR Biomarker score to determine if a subject from which the sample was obtained is selected to receive one or more doses of a TTR stabilizing agent.

In some embodiments, a subject is a human subject.

In some embodiments, a sample includes blood, serum, plasma, or cardiac tissue.

The present disclosure also provides a non-transitory computer readable medium. In some embodiments, a non-transitory computer readable medium contains executable instructions that when executed cause a processor to perform operations including a method described herein.

In addition, the present disclosure provides compositions. In some embodiments, a composition includes one or more ATTR Biomarkers. In some embodiments, one or more ATTR Biomarkers include TnI, PKM1, PKM2, NT-proBNP, RBP4, TIMP2, NfL, or a combination thereof.

In some embodiments, a composition includes one or more anti-ATTR Biomarker agents. In some embodiments, one or more anti-ATTR Biomarker agents include an anti-TnI agent, an anti-PKM1 agent, an anti-PKM2 agent, an anti-NT-proBNP agent, an anti-RBP4 agent, an anti-TIMP2 agent, an anti-NfL agent, or a combination thereof.

The present disclosure further provides kits. In some embodiments, a kit includes one or more ATTR Biomarkers. In some embodiments, one or more ATTR Biomarkers include TnI, PKM1, PKM2, NT-proBNP, RBP4, TIMP2, NfL, or a combination thereof.

In some embodiments, a kit includes one or more anti-ATTR Biomarker agents. In some embodiments, one or more anti-ATTR Biomarker agents include an anti-TnI agent, an anti-PKM1 agent, an anti-PKM2 agent, an anti-NT-proBNP agent, an anti-RBP4 agent, an anti-TIMP2 agent, an anti-NfL agent, or a combination thereof.

In some embodiments, a kit includes instructions for use.

In some embodiments, a kit includes one or more anti-ATTR Biomarker agents, where the one or more anti-ATTR Biomarkers include one or more antibody agents. In some embodiments, one or more of the antibody agents are labeled with a detectable moiety.

In some embodiments, a kit includes one or more control samples. In some embodiments, control samples include one or more ATTR Biomarker standards.

The present disclosure provides uses of a kit described herein. In some embodiments, a kit can be used in an in vitro diagnostic assay to diagnose TTR-CM in a subject.

### BRIEF DESCRIPTION OF THE DRAWING

FIG. 1 shows patient cohorts used in exemplary methods described herein.

FIG. 2 shows a summary of data resulting from exemplary methods described herein.

FIG. 3A includes a graph showing the individual sensitivity and specificity performance determined with TIMP2, and FIG. 3B includes a graph showing the individual sensitivity and specificity performance determined with TnI.

FIG. 4A includes a graph showing the individual sensitivity and specificity performance determined with PKM, and FIG. 4B includes a graph showing the individual sensitivity and specificity performance determined with RBP4.

FIG. 5A includes a graph showing the individual sensitivity and specificity performance determined with RBP, and FIG. 5B includes a graph showing the individual sensitivity and specificity performance determined with DCN.

FIG. 6A includes a graph showing the individual sensitivity and specificity performance determined with NT-proBNP, and FIG. 6B includes a graph showing the individual sensitivity and specificity performance determined with SMOC-2.

FIG. 7 includes a bar graph showing representative biomarker effects for each NYHA class.

FIG. 8 includes a table providing the results of screens with exemplary biomarkers described herein, including an assessment of regression fits used.

FIG. 9 includes tables summarizing data obtained when select subsets of biomarkers were used in various predictive models.

FIG. 10 presents optimization of detection cutoff values for assays measuring PKM, TIMP2, LIMS-1, C3, and A11. FIG. 10A depicts ROC plot data for optimization of detection cutoff values. FIG. 10B depicts exemplary values for sensitivity, specificity, accuracy and detection cutoff values for the listed biomarkers.

FIG. 11 depicts an exemplary block diagram of a computer system 1100.

FIG. 12 depicts an exemplary flow chart of a method 1200.

FIG. 13 depicts an exemplary flow chart of a method 1300.

FIG. 14 includes a table showing the S/N ratios of selected markers.

### DEFINITIONS

Antibody agent: As used herein, the term “antibody agent” refers to an agent that specifically binds to a particular antigen. In some embodiments, the term encompasses any polypeptide or polypeptide complex that includes immunoglobulin structural elements sufficient to confer specific binding. Such polypeptide may be naturally produced (e.g., generated by an organism reacting to an antigen), or produced by recombinant engineering, chemical synthesis, or other artificial system or methodology. Exemplary antibody agents include, but are not limited to, human antibodies, primatized antibodies, chimeric antibodies, bi-specific antibodies, humanized antibodies, conjugated antibodies (e.g., antibodies conjugated or fused to other proteins, radiolabels, cytotoxins), Small Modular ImmunoPharmaceuticals (“SMIPs™”), single chain antibodies, cameloid antibodies, and antibody fragments. As used herein, the term “antibody agent” also includes intact monoclonal antibodies, polyclonal antibodies, single domain antibodies (e.g., shark single domain antibodies (e.g., IgNAR or fragments thereof)), multispecific antibodies (e.g. bi-specific antibodies) formed from at least two intact antibodies, and antibody fragments so long as they exhibit the desired biological activity. An antibody agent can have antibody constant region sequences that are characteristic of mouse, rabbit, primate, or human antibodies. In some embodiments, the term encompasses stapled peptides. In some embodiments, the term encompasses one or more antibody-like binding peptidomimetics. In some embodiments, the term encompasses one or more antibody-like binding scaffold proteins. In some embodiments, the term encompasses monobodies or adnectins. In many embodiments, an antibody agent is or includes a polypeptide whose amino acid sequence includes one or more structural elements recognized by those skilled in the art as a complementarity determining region (CDR); in some embodiments an antibody agent is or includes a polypeptide whose amino acid sequence includes at least one CDR (e.g., at least one heavy chain CDR and/or at least one light chain CDR) that is substantially identical to one found in a reference antibody. In some embodiments, an antibody agent is or includes a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain. In some embodiments, an antibody agent may contain a covalent modification (e.g., attachment of a glycan, a payload (e.g., a detectable moiety, a therapeutic moiety, a catalytic moiety, etc.), or other pendant group (e.g., poly-ethylene glycol, etc.).

Biomarker: The term “biomarker” or “biological marker” is used herein, consistent with its use in the art, to refer to an entity whose presence, level, or form, correlates with a particular biological event or state of interest, so that it is considered to be a “marker” of that event or state. To give but a few examples, in some embodiments, a biomarker may be or include a marker for a particular disease state, or for likelihood that a particular disease, disorder or condition may develop, occur, or reoccur. In some embodiments, a biomarker may be or include a marker for a particular disease or therapeutic outcome, or likelihood thereof. Thus, in some embodiments, a biomarker is predictive, in some embodiments, a biomarker is prognostic, in some embodiments, a biomarker is diagnostic, of the relevant biological event or state of interest. In some embodiments, a biomarker is a possible biomarker of the relevant biological event or state of interest. A biomarker may be an entity of any chemical class. For example, in some embodiments, a biomarker may be or include a nucleic acid, a polypeptide, a small molecule, or a combination thereof. In some embodiments, a biomarker is a cell surface marker. In some embodiments, a biomarker is intracellular. In some embodiments, a biomarker is found in a particular tissue (e.g., cardiac tissue). In some embodiments, a biomarker is found outside of cells (e.g., is secreted or is otherwise generated or present outside of cells, e.g., in a body fluid such as blood, urine, tears, saliva, cerebrospinal fluid, etc.

As described herein, in some embodiments, a biomarker is an ATTR Biomarker. An “ATTR Biomarker” as used herein refers to a biological marker for ATTR amyloidosis or TTR-CM. In some embodiments, one or more one or more ATTR Biomarkers include TnI, PKM1, PKM2, NT-proBNP, RBP4, DCN, TIMP2, SMOC-2, NfL, or a combination thereof. For avoidance of doubt, an ATTR Biomarker includes a gene product associated with the specific recited biomarker. For example, depending on context, “TnI” refers to a nucleotide encoding TnI or a characteristic fragment thereof, as well as a TnI protein or a characteristic fragment thereof.

Characteristic fragment: The term “characteristic fragment” refers to a fragment of a biomarker (e.g., ATTR Biomarker) that is sufficient to identify the biomarker from which the fragment was derived. For example, in some embodiments, a “characteristic fragment” of a biomarker is one that contains an amino acid sequence, or a collection of amino acid sequences, that together allow for the biomarker from which the fragment was derived to be distinguished from other possible biomarkers, proteins, or polypeptides. In some embodiments, a characteristic fragment includes at least 10, at least 20, at least 30, at least 40, or at least 50 amino acids.

Gene product or expression product: As used herein, the term “gene product” generally refers to an RNA transcribed from the gene (pre- and/or post-processing) or a polypeptide (pre- and/or post-modification) encoded by an RNA transcribed from the gene.

Hybridization: The term “hybridization” refers to the physical property of single-stranded nucleic acid molecules (e.g., DNA or RNA) to anneal to complementary nucleic acid molecules. Hybridization can typically be assessed in a variety of contexts-including where interacting nucleic acid molecules are studied in isolation or in the context of more complex systems (e.g., while covalently or otherwise associated with a carrier entity and/or in a biological system or cell). In some embodiments, hybridization can be detected by a hybridization technique, such as a technique selected from the group consisting of in situ hybridization (ISH), microarray, Northern blot, and Southern blot. In some embodiments, hybridization refers to 100% annealing between the single-stranded nucleic acid molecules and the complementary nucleic acid molecule. In some embodiments, annealing is less than 100% (e.g., at least 95%, at least 90%, at least 85%, at least 80%, at least 75%, at least 70% of a single-stranded nucleic acid molecule anneals to a complementary nucleic acid molecule). Hybridization techniques, and methods for evaluating hybridization, are well known in the art. See, e.g., Sambrook, et al., 1989, Molecular Cloning: A Laboratory Manual, Second Edition, Cold Spring Harbor Press, Plainview, N.Y. Those skilled in the art understand how to estimate and adjust the stringency of hybridization conditions such that sequences having at least a desired level of complementary will stably hybridize, while those having lower complementary will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology. John Wiley & Sons, Secaucus, N.J.

Detection agent: The term “detection agent” as used herein refers to any element, molecule, functional group, compound, fragment or moiety that is detectable. In some embodiments, a detection agent is provided or utilized alone. In some embodiments, a detection agent is provided and/or utilized in association with (e.g., joined to) another agent. Examples of detection agents include, but are not limited to: various ligands, radionuclides (e.g., ³H, ¹⁴C, ¹⁸F, ¹⁹F, ³²P, ³⁵S, ¹³⁵I, ¹²⁵I, ¹²³I, ⁶⁴Cu, ¹⁸⁷Re, ¹¹¹In, ⁹⁰Y, ^{99m}Tc, ¹⁷⁷Lu, ⁸⁹Zr etc.), fluorescent dyes, chemiluminescent agents (such as, for example, acridinum esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes, colorimetric labels (such as, for example, dyes, colloidal gold, and the like), biotin, dioxigenin, haptens, and proteins for which antisera or monoclonal antibodies are available.

Diagnostic test: As used herein, “diagnostic test” is a step or series of steps that is or has been performed to attain information that is useful in determining whether a patient has a disease, disorder or condition and/or in classifying a disease, disorder or condition into a phenotypic category or any category having significance with regard to prognosis of a disease, disorder or condition, or likely response to treatment (either treatment in general or any particular treatment) of a disease, disorder or condition. Similarly, “diagnosis” refers to providing any type of diagnostic information, including, but not limited to, whether a subject is likely to have or develop a disease, disorder or condition, state, staging or characteristic of a disease, disorder or condition as manifested in the subject, information related to the nature or classification of a tumor, information related to prognosis and/or information useful in selecting an appropriate treatment or additional diagnostic testing. Selection of treatment may include the choice of a particular therapeutic agent or other treatment modality such as surgery, radiation, etc., a choice about whether to withhold or deliver therapy, a choice relating to dosing regimen (e.g., frequency or level of one or more doses of a particular therapeutic agent or combination of therapeutic agents), etc. Selection of additional diagnostic testing may include more specific testing for a given disease, disorder, or condition.

Sample: As used herein, the term “sample” refers to a biological sample obtained or derived from a human subject, as described herein. In some embodiments, a biological sample includes biological tissue or fluid. In some embodiments, a biological sample may include blood; blood cells; tissue or fine needle biopsy samples; cell-containing body fluids; free floating nucleic acids; cerebrospinal fluid; lymph; tissue biopsy specimens; surgical specimens; other body fluids, secretions, and/or excretions; and/or cells therefrom. In some embodiments, a biological sample includes cells obtained from an individual, e.g., from a human or animal subject. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood). In some embodiments, a sample is cardiac tissue obtained from the subject. In some embodiments, as will be clear from context, the term “sample” refers to a preparation that is obtained by processing (e.g., by removing one or more components of and/or by adding one or more agents to) a primary sample. For example, filtering using a semi-permeable membrane. As another example of sample processing, the sample may be a plasma sample that is treated with an anticoagulant selected from the group consisting of EDTA, heparin, and citrate. As another example of sample processing, the sample may be processed to isolate one or more proteins (e.g., by capturing proteins with one or more antibodies). A “processed sample” may include, for example, nucleic acids or polypeptides extracted from a sample or obtained by subjecting a primary sample to techniques such as amplification or reverse transcription of mRNA, isolation and/or purification of certain components.

Subject: As used herein, the term “subject” refers to an organism, for example, a mammal (e.g., a human). In some embodiments a human subject is an adult, adolescent, or pediatric subject. In some embodiments, a subject is at least 50, at least 55, at least 60, at least 65, at least 70, at least 75, or at least 80 years of age. In some embodiments, a subject is suffering from a disease, disorder or condition, e.g., a disease, disorder or condition that can be treated as provided herein. In some embodiments, a subject is susceptible to a disease, disorder, or condition; in some embodiments, a susceptible subject is predisposed to and/or shows an increased risk (as compared to the average risk observed in a reference subject or population) of developing the disease, disorder or condition. In some embodiments, a subject displays one or more symptoms of a disease, disorder or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered.

Threshold value: As used herein, the term “threshold value” refers to a value (or values) that are used as a reference to attain information on and/or classify the results of a measurement, for example, the results of a measurement attained in an assay. A threshold value can be determined based on one or more control samples. A threshold value can be determined prior to, concurrently with, or after the measurement of interest is taken. In some embodiments, a threshold value can be a range of values. In some embodiments, a threshold value can be a value (or range of values) reported in the relevant field (e.g., a value found in a standard table).

### DETAILED DESCRIPTION OF CERTAIN EMBODIMENTS

### Amyloid Transthyretin Cardiomyopathy

Transthyretin (TTR) is a transport protein in the serum and cerebrospinal fluid that carries the thyroid hormone thyroxin (T₄) and retinol-binding protein. The liver secretes TTR into the blood and the choroid plexus secretes TTR into the cerebrospinal fluid. TTR is produced as a homotetrameric complex. However, TTR can undergo conformational transformation to aggregate into abnormal amyloid form leading to pathologic conditions.

The condition of ATTR amyloidosis is characterized by the deposition of amyloid fibrils, derived from transthyretin (TTR) protein, in various organs and tissues. For example, misfolding of TTR protein can cause amyloid fibril deposition in heart tissue and lead to cardiomyopathy (referred to here as “amyloid transthyretin cardiomyopathy” or “TTR-CM”). Clinical symptoms of TTR-CM include increasing ventricular wall thickness and heart failure.

There are three types of ATTR amyloidosis: (1) familial amyloid polyneuropathy (FAP), (2) familial amyloid cardiomyopathy (FAC), and (3) senile systemic amyloidosis, which is also called wild-type ATTR amyloidosis (ATTRwt). Familial amyloid polyneuropathy (FAP) affects the nervous system, in addition to the heart and sometimes the kidneys and eyes. Symptoms of FAP may include peripheral neuropathy, autonomic neuropathy, and heart failure. Familial amyloid cardiomyopathy (FAC) affects the heart and also manifests with carpal tunnel syndrome. FAP and FAC are hereditary conditions caused by mutations in the TTR gene that lead to production of abnormal (“variant”) TTR. Over 100 different mutations in the TTR gene have been observed, most of which cause production of variant TTR that can misfold into amyloid fibrils and cause aggregates of amyloid deposits in tissues. Most affected individuals are heterozygotes; thus, both mutant and wild-type TTR can be included in aggregates. As used herein, hereditary cardiomyopathy resulting from mutant or variant forms of TTR is referred to as familial amyloid cardiomyopathy (abbreviated ATTRm). Wild-type ATTR amyloidosis (ATTRwt) is a slowly-progressive, non-hereditary (sporadic) disease. Individuals with ATTRwt do not have a mutation in the TTR gene and the amyloid fibrils consist of wild-type TTR. Symptoms of ATTRwt include heart failure and, in some individuals, carpal tunnel syndrome. ATTRwt occurs more often in aged subjects 65 years or older.

While accurate prevalence is unknown, a rough estimate at 0.4 cases/million people/year in the United States has been given for ATTRm, and at least a two-fold higher incidence for ATTRwt. However, autopsy studies of patients over 80 years of age have shown a prevalence of about 25% for ATTRwt, suggesting that the disease is largely underdiagnosed and the actual prevalence, particularly in the elderly population is much higher. The challenge of testing numerous individuals with costly and invasive tests and procedures is a likely contributing factor to significant underdiagnoses of TTR-CM.

### Current Diagnosis of Amyloid Transthyretin Cardiomyopathy

TTR-CM is currently diagnosed using a series of different tests that begin with an echocardiogram (Gertz, M. A. et al. JACC 66:2452-2466, 2015). This technique is used for determining general heart function and looking for structural abnormalities (Ashley, E. A. and Niebauer, J., Cardiology Explained, London: Remedica, Chapter 4, 2004) and is used as a screen for cardiac amyloidosis as evidenced by thickening and hypertrophy of the heart ventricles. Although the test is not able to distinguish between hypertropic and hypertensive cardiomyopathy, it has shown relatively good specificity (e.g., 82%) in differentiating cardiac amyloidosis from cardiac hypertrophy in one study (Ashley 2004). However, because there are more than one form of cardiac amyloidosis (e.g., Light Chain Amyloidosis (AL)), this test is not specific for TTR-CM.

Patients that test positive with an echocardiogram are next tested by cardiac magnetic resonance imaging (CMR) (Gertz, M. A. et al. JACC 66:2452-2466, 2015; Krishnamurthy, R. et al. Current Cardiology Reviews, 9:185-190, 2013; Doltra, A. et al. Curr Cardiol Rev. 9(3):185-90, 2013). This technique uses the contrast agent gadolinium which is able to concentrate in areas of cardiac cellular damage (e.g., myocardial infarction) or in areas where the extracellular space has increased due to scarring or amyloid deposits (Krishnamurthy, R. et al. Current Cardiology Reviews, 9:185-190, 2013). The technique is better able to distinguish between hypertensive and hypertropic cardiomyopathy than an echocardiogram and in one study has shown greater specificity for detecting ATTR amyloidosis over AL (Ashley 2004). However, this test on its own is also not a definitive test for TTR-CM diagnosis.

Another imaging method for the detection of cardiac amyloidosis is scintigraphy, using radioisotope conjugates such as 99mTc-Pyrophosphate (Bokhari et al. Circ Cardiovasc Imaging. 6(2):195-201, 2013). This is performed using single photon emission computed tomography (SPECT) to gain a 3D image and while the radioactive tracer is not specific to heart tissue it is useful in detecting areas of poor blood flow, such as occurs in diseased heart tissue. This technique has been shown in one study to differentiate ATTR amyloidosis from AL amyloidosis with high specificity and sensitivity (100% and 97%, respectively).

Currently, the most definitive test for diagnosis of TTR-CM, which is typically conducted only after positive scores have been obtained using the above mentioned tests, is a cardiac biopsy followed by immunochemical staining. Polyclonal antibodies to kappa or light chain amyloid deposits in heart tissue are used for detection of AL, while polyclonal antibodies to transthyretin deposits are used for the detection of TTR-CM (Crotty, T. B., et al. Cardiovacular Pathology 4: 39-42, 1995).

Finally, antibodies for transthyretin (TTR) have previously been disclosed and applications of such antibodies in the diagnosis of amyloid diseases such as ATTRwt have been described (WO 2014/124334 A2 and WO 2016/120811). The disadvantages to using such anti-TTR antibodies for diagnosis of cardiomyopathies such as cardiomyopathy resulting from ATTRwt is that TTR may be unrecognizable in a misfolded or aggregate state in some patients, resulting in false negative test results and underdiagnosis. For example, both WO 2014/124334 A2 and WO 2016/120811 rely on exposure of epitopes within specific amino acid residues of TTR; however, such epitopes may not be readily accessible in all ATTR amyloidosis patients depending on the non-native form of TTR. Wild-type TTR, mutant TTR, or mixed TTR tetramers can dissociate, misfold, aggregate and/or form fibrils in ATTR amyloidosis disease. Such different forms may not be readily detected by anti-TTR antibodies. Additionally, the gene encoding TTR is reported to have many different mutations associated with ATTR amyloidosis. Therefore, anti-TTR antibodies may not be able to recognize disease in many patients.

### ATTR Biomarkers

Embodiments described herein provide a number of advantages over the prior techniques discussed herein. For example, technologies of the present disclosure are non-invasive, require minimal patient discomfort, are quick to perform, and are relatively cost-effective. Accordingly, technologies described herein offer advantages over these prior techniques including, but not limited to providing: a non-invasive in vitro diagnostic (IVD) test for TTR-CM resulting from ATTRwt, one or more specific in vitro biomarkers suitable for use in an IVD testing, alternatives to a single marker IVD test including more than one marker to effectively rule in or rule out candidates for the more costly and invasive procedures in the diagnosis of the disease.

The present disclosure relates particularly to biomarkers for ATTR amyloidosis and TTR-CM. Such biomarkers are referred to herein as “ATTR Biomarkers.” ATTR Biomarkers can include, for example, TnI, PKM1, PKM2, NT-proBNP, RBP4, DCN, TIMP2, SMOC-2, NfL, characteristic fragments thereof, and/or variants thereof.

As provided herein, an ATTR Biomarker includes gene products associated with the specific recited biomarker. For example, ATTR Biomarkers can include, for example, a protein or nucleotide (e.g., RNA, e.g., mRNA). ATTR Biomarkers also encompass full-length proteins, as well as fragments (e.g., characteristic fragments) of an ATTR Biomarker. For example, in some embodiments, an ATTR Biomarker can include a protein as listed in Table 1. In some embodiments, an ATTR Biomarker includes a fragment having an amino acid sequence identical to a contiguous span of at least 10 amino acids, at least 20 amino acids, at least 30 amino acids, at least 40 amino acids, at least 50 amino acids, at least 60 amino acids, at least 70 amino acids, at least 80 amino acids, at least 90 amino acids, or at least 100 amino acids of an amino acid sequence provided in Table 1. In some embodiments, an ATTR Biomarker includes a fragment having at least 70%, at least 75%, at least 80%, at least 85%, at least 90%, at least 95%, at least 96%, at least 97%, at least 98%, or at least 99% identity to an amino acid sequence provided in Table 1. Variant or alternative forms of the biomarker include for example polypeptides encoded by any splice-variants of transcripts encoding the disclosed biomarkers.

Biomarkers contemplated herein also include truncated forms or polypeptide fragments of any of the proteins described herein. Truncated forms or polypeptide fragments of a protein can include N-terminally deleted or truncated forms and C-terminally deleted or truncated forms. Truncated forms or fragments of a protein can include fragments arising by any mechanism, such as, without limitation, by alternative translation, exo- and/or endo-proteolysis and/or degradation, for example, by physical, chemical and/or enzymatic proteolysis. Without limitation, a biomarker may include a truncated or fragment of a protein, polypeptide or peptide may include at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 8%, or at least 99% of the amino acid sequence of an ATTR Biomarker protein.

In some instances, a fragment is N-terminally and/or C-terminally truncated by 1-20 amino acids, such as, for example, by 1-15 amino acids, by 1-10 amino acids, or by 1-5 amino acids, compared to the corresponding mature, full-length ATTR Biomarker protein.

Any ATTR Biomarker protein of the present disclosure such as a peptide, polypeptide or protein and fragments thereof may also encompass modified forms of said marker, peptide, polypeptide or protein and fragments such as bearing post-expression modifications including but not limited to, modifications such as phosphorylation, glycosylation, lipidation, methylation, selenocystine modification, cysteinylation, sulphonation, glutathionylation, acetylation, and/or oxidation of methionine to methionine sulphoxide or methionine sulphone.

In some embodiments, an ATTR Biomarker has different isoforms. Although only one or more isoforms may be disclosed herein, all isoforms of the ATTR Biomarkers are contemplated for use in the disclosed technologies.

In some embodiments, an ATTR Biomarker can be a nucleotide. In some embodiments, a nucleotide can be RNA or DNA. In some cases, corresponding RNA or DNA may exhibit better discriminatory power in diagnosis than the full-length protein.

Exemplary ATTR Biomakers for use with technologies provided herein are described briefly below.

### Troponin I (TnI)

Troponins are a group of proteins found in skeletal and cardiac muscle fibers. One function of troponins is to regulate muscular contraction. Three types of troponin proteins are known: troponin C, troponin I, and troponin T. Together, the three types of troponin form of a complex. Within the complex, troponin C binds to calcium ions. This binding initiates contraction by producing a conformational change in troponin I. Troponin I binds to actin in thin myofilaments to hold the actin-tropomyosin complex in place. Troponin T anchors the troponin complex to tropomyosin, a muscle fiber structure.

Prior analysis shows that there is little or no difference in troponin C between skeletal and cardiac muscle, while forms of troponin I and troponin T are understood to be different between skeletal and cardiac muscle. Normally, troponin is present in very small to undetectable quantities in the blood. However, when there is damage to heart muscle cells, troponin is released into the blood. A greater concentration of troponin I in the blood generally correlates with more damage to cardiac tissue.

In some embodiments of the present disclosure, TnI is an ATTR Biomarker. In some embodiments, detection of TnI, a characteristic fragment of TnI, and/or a variant of TnI is used in a method for assessing a subject's risk of developing TTR-CM, diagnosing a subject with TTR-CM or recommending a subject for additional cardiomyopathy testing. In some embodiments, TnI is detected in a sample with anti-TnI agents (e.g., anti-TnI antibody agents, probes, etc.). In some embodiments, detection of nucleotides that encode TnI, nucleotides that encode characteristic fragments TnI, and/or nucleotides that encode variants of TnI is used in a method for assessing a subject's risk of developing TTR-CM, diagnosing a subject with TTR-CM or recommending a subject for additional cardiomyopathy testing. In some embodiments, nucleotides that encode TnI are detected in a sample with anti-TnI nucleotide sequence agents (e.g., anti-TnI nucleotide sequence antibody agents, probes, complementary nucleic acids, etc.).

### Pyruvate Kinase Muscle Isoform 1 and Isoform 2 (PKM1 and PKM2)

Pyruvate kinase is an enzyme that catalyzes the final step in glycolysis by catalyzing the transfer of a phosphate group from phosphoenolpyruvate (PEP) to adenosine diphosphate (ADP), yielding pyruvate and ATP. In vertebrates, there are four tissue-specific isozymes of pyruvate kinase: L (liver), R (erythrocytes), muscle isoform 1 (muscles, heart and brain) and muscle isoform 2 (early fetal tissue and most adult tissues). Pyruvate kinase protein can form dimers and tetramers.

The PKM gene encodes the muscle isoform 1 and muscle isoform 2 isozymes (PKM1 and PKM2). Exons 9 and 10 of the PKM gene contain sequences for the muscle isoform 1 and muscle isoform 2 isozymes, respectively. There are at least 14 splice variants of PKM, including 1 noncoding variant. Among the splice variants of PKM are PKM1 and PKM2, which are produced by differential splicing and differ by 22 amino acids at their carboxy termini. Because the amino acid sequences of PKM1 and PKM2 share regions of identity, certain fragments of PKM1 and PKM2 will be characteristic fragments of both PKM1 and PKM2, and certain fragments (e.g., fragments from the 22 amino acids at the carboxy terminus) will be characteristic fragments of PKM1 or PKM2. Additionally, because the amino acid sequences of PKM1 and PKM2 share regions of identity, certain anti-PKM1 agents will also detect PKM2, and vice versa.

In some embodiments of the present disclosure, PKM (e.g., PKM1 and/or PKM2) is an ATTR Biomarker. In some embodiments, detection of PKM, a characteristic fragment of PKM, and/or a variant of PKM is used in a method for assessing a subject's risk of developing TTR-CM, diagnosing a subject with TTR-CM or recommending a subject for additional cardiomyopathy testing. In some embodiments, PKM is detected in a sample with anti-PKM agents (e.g., anti-PKM antibody agents, probes, etc.). In some embodiments, detection of nucleotides that encode PKM, nucleotides that encode a characteristic fragment of PKM, and/or nucleotides that encode variants of PKM is used in a method for assessing a subject's risk of developing TTR-CM, diagnosing a subject with TTR-CM or recommending a subject for additional cardiomyopathy testing. In some embodiments, nucleotides that encode PKM are detected in a sample with anti-PKM nucleotide sequence agents (e.g., anti-PKM nucleotide sequence antibody agents, probes, complementary nucleic acids, etc.).

In some embodiments of the present disclosure, PKM1 is an ATTR Biomarker. In some embodiments, detection of PKM1, a characteristic fragment of PKM1, and/or a variant of PKM1 is used in a method for assessing a subject's risk of developing TTR-CM, diagnosing a subject with TTR-CM or recommending a subject for additional cardiomyopathy testing. In some embodiments, PKM1 is detected in a sample with anti-PKM1 agents (e.g., anti-PKM1 antibody agents, probes, etc.). In some embodiments, detection of nucleotides that encode PKM1, nucleotides that encode a characteristic fragment of PKM1, and/or nucleotides that encode variants of PKM1 is used in a method for assessing a subject's risk of developing TTR-CM, diagnosing a subject with TTR-CM or recommending a subject for additional cardiomyopathy testing. In some embodiments, nucleotides that encode PKM1 are detected in a sample with anti-PKM1 nucleotide sequence agents (e.g., anti-PKM1 nucleotide sequence antibody agents, probes, complementary nucleic acids, etc.).

In some embodiments of the present disclosure, PKM2 is an ATTR Biomarker. In some embodiments, detection of PKM2, a characteristic fragment of PKM2, and/or a variant of PKM2 is used in a method for assessing a subject's risk of developing TTR-CM, diagnosing a subject with TTR-CM or recommending a subject for additional cardiomyopathy testing. In some embodiments, PKM2 is detected in a sample with anti-PKM2 agents (e.g., anti-PKM2 antibody agents, probes, etc.). In some embodiments, detection of nucleotides that encode PKM2, nucleotides that encode a characteristic fragment of PKM2, and/or nucleotides that encode variants of PKM2 is used in a method for assessing a subject's risk of developing TTR-CM, diagnosing a subject with TTR-CM or recommending a subject for additional cardiomyopathy testing. In some embodiments, nucleotides that encode PKM2 are detected in a sample with anti-PKM2 nucleotide sequence agents (e.g., anti-PKM2 nucleotide sequence antibody agents, probes, complementary nucleic acids, etc.).

### N-Terminal-Pro Hormone B-Type Natriuretic Peptide (NT-proBNP)

B-type natriuretic peptide (BNP) is a hormone produced by your heart. BNP is a small, ringed peptide secreted by the heart to regulate blood pressure and fluid balance. N-terminal (NT)-pro hormone BNP (NT-proBNP or NT-proBNP) is a non-active prohormone that is released from the same molecule that produces BNP. In particular, BNP is stored in and secreted predominantly from membrane granules in the heart ventricles in a pro form (proBNP). Once released from the heart in response to ventricle volume expansion or pressure overload, the N-terminal (NT) piece of 76 amino acids (NT-proBNP) is rapidly cleaved by the enzymes corin and furin to release the active 32-amino acid peptide (BNP).

In some embodiments of the present disclosure, NT-proBNP is an ATTR Biomarker. In some embodiments, detection of NT-proBNP, a characteristic fragment of NT-proBNP, and/or a variant of NT-proBNP is used in a method for assessing a subject's risk of developing TTR-CM, diagnosing a subject with TTR-CM or recommending a subject for additional cardiomyopathy testing. In some embodiments, NT-proBNP is detected in a sample with anti-NT-proBNP agents (e.g., anti-NT-proBNP antibody agents, probes, etc.). In some embodiments, detection of nucleotides that encode NT-proBNP, nucleotides that encode a characteristic fragment of NT-proBNP, and/or nucleotides that encode variants of NT-proBNP is used in a method for assessing a subject's risk of developing TTR-CM, diagnosing a subject with TTR-CM or recommending a subject for additional cardiomyopathy testing. In some embodiments, nucleotides that encode NT-proBNP are detected in a sample with anti-NT-proBNP nucleotide sequence agents (e.g., anti-NT-proBNP nucleotide sequence antibody agents, probes, complementary nucleic acids, etc.).

### Retinol Binding Protein 4 (RBP4)

Retinol binding protein 4 (RBP4) is a transporter protein for retinol (vitamin A alcohol). RBP4 has a molecular weight of approximately 21 kDa and is encoded by the RBP4 gene in humans. It is mainly, though not exclusively, synthesized in the liver. RBP4 delivers retinol from liver stores to the peripheral tissues. In plasma, RBP-retinol complexes interact with transthyretin, which prevents its loss by filtration through the kidney glomeruli. A deficiency of vitamin A blocks secretion of the binding protein posttranslationally and results in defective delivery and supply to the epidermal cells. Circulating RBP4 has previously been proposed as a way to discriminate ATTRm from non-amyloid heart failure (Arvanitis, M. et al., JAMA Cardiol., 2017). However, this prior study only examined ATTRm caused by a particular mutation in RBP4 (substitution of valine for isoleucine at codon 122 of the TTR gene (V122I)), and did not examine whether RBP4 could be used more generally as a biomarker for TTR-CM, whether assessed alone or with other possible biomarkers.

The present disclosure provides the recognition that RBP4 can be used as an ATTR Biomarker. Accordingly, in some embodiments of the present disclosure, RBP4 is an ATTR Biomarker. In some embodiments, detection of RBP4, a characteristic fragment of RBP4, and/or a variant of RBP4 is used in a method for assessing a subject's risk of developing TTR-CM, diagnosing a subject with TTR-CM or recommending a subject for additional cardiomyopathy testing. In some embodiments, RBP4 is detected in a sample with anti-RBP4 agents (e.g., anti-RBP4 antibody agents, probes, etc.). In some embodiments, detection of nucleotides that encode RBP4, nucleotides that encode a characteristic fragment of RBP4, and/or nucleotides that encode variants of RBP4 is used in a method for assessing a subject's risk of developing TTR-CM, diagnosing a subject with TTR-CM or recommending a subject for additional cardiomyopathy testing. In some embodiments, nucleotides that encode RBP4 are detected in a sample with anti-RBP4 nucleotide sequence agents (e.g., anti-RBP4 nucleotide sequence antibody agents, probes, complementary nucleic acids, etc.).

### Tissue Inhibitor of Metalloproteinases 2 (TIMP2)

Tissue inhibitor of metalloproteinases 2 (TIMP2) is a gene that encodes the TIMP2 protein. The TIMP2 gene is encoded by 5 exons spanning 83 kb of genomic DNA. The 5-prime end of the TIMP2 gene contains several regulatory elements, including Sp1-, AP2-, AP1-, and PEA3-binding sites.

The TIMP2 gene is a member of the TIMP gene family. Proteins encoded by genes of the TIMP gene family inhibit matrix metalloproteinases (MMP), a group of peptidases involved in degradation of the extracellular matrix. TIMP2 also has the ability to directly suppress the proliferation of endothelial cells. TIMP2 has been shown to suppress tumor metastasis.

In some embodiments of the present disclosure, TIMP2 is an ATTR Biomarker. In some embodiments, detection of TIMP2, a characteristic fragment of TIMP2, and/or a variant of TIMP2 is used in a method for assessing a subject's risk of developing TTR-CM, diagnosing a subject with TTR-CM or recommending a subject for additional cardiomyopathy testing. In some embodiments, TIMP2 is detected in a sample with anti-TIMP2 agents (e.g., anti-TIMP2 antibody agents, probes, etc.). In some embodiments, detection of nucleotides that encode TIMP2 nucleotides that encode a characteristic fragment of TIMP2, and/or nucleotides that encode variants of TIMP2 is used in a method for assessing a subject's risk of developing TTR-CM, diagnosing a subject with TTR-CM or recommending a subject for additional cardiomyopathy testing. In some embodiments, nucleotides that encode TIMP2 are detected in a sample with anti-TIMP2 nucleotide sequence agents (e.g., anti-TIMP2 nucleotide sequence antibody agents, probes, complementary nucleic acids, etc.).

### Neurofilament Light Chain (NfL)

Neurofilaments are cytoskeletal components of neurons that are particularly abundant in axons. The functions of neurofilaments include provision of structural support and maintenance of the size, shape, and caliber of axons. Neurofilaments include three subunits: neurofilament light chain (NfL), neurofilament medium chain, and neurofilament heavy chain. NfL levels increase in cerebrospinal fluid (CSF) and blood proportionally to the degree of axonal damage in a variety of neurological disorders, including inflammatory, neurodegenerative, traumatic and cerebrovascular diseases. While NfL has been used as a biomarker for neurodegenerative disorders, its connection to other diseases and conditions, including cardiac conditions, has not been fully explored.

As described herein, in some embodiments, neurofilament light chain (NfL) can be useful for detecting and diagnosing TTR-CM. In some embodiments, NfL is an ATTR Biomarker. In some embodiments, detection of NfL, a characteristic fragment of NfL, and/or a variant of NfL is used in a method for assessing a subject's risk of developing TTR-CM, diagnosing a subject with TTR-CM or recommending a subject for additional cardiomyopathy testing. In some embodiments, NfL is detected in a sample with anti-NfL agents (e.g., anti-NfL antibody agents, probes, etc.). In some embodiments, detection of nucleotides that encode NfL nucleotides that encode a characteristic fragment of NfL, and/or nucleotides that encode variants of NfL is used in a method for assessing a subject's risk of developing TTR-CM, diagnosing a subject with TTR-CM or recommending a subject for additional cardiomyopathy testing. In some embodiments, nucleotides that encode NfL are detected in a sample with anti-NfL nucleotide sequence agents (e.g., anti-NfL nucleotide sequence antibody agents, probes, complementary nucleic acids, etc.).

### Decorin (DCN)

Decorin (DCN) is a proteoglycan that is on average 90-140 kilodaltons (kDa) in molecular weight. It belongs to the small leucine-rich proteoglycan (SLRP) family and includes a protein core containing leucine repeats with a glycosaminoglycan (GAG) chain of either chondroitin sulfate (CS) or dermatan sulfate (DS). DCN is a component of connective tissue, binds to type I collagen fibrils. DCN also acts as a ligand of various cytokines and growth factors by directly or indirectly interacting with corresponding signalling molecules involved in cell growth, differentiation, proliferation, adhesion and metastasis and that DCN especially plays vital roles in cancer cell proliferation, spread, pro-inflammatory processes and anti-fibrillogenesis.

In some embodiments of the present disclosure, DCN is an ATTR Biomarker. In some embodiments, detection of DCN, a characteristic fragment of DCN, and/or a variant of DCN is used in a method for assessing a subject's risk of developing TTR-CM, diagnosing a subject with TTR-CM or recommending a subject for additional cardiomyopathy testing. In some embodiments, DCN is detected in a sample with anti-DCN agents (e.g., anti-DCN antibody agents, probes, etc.). In some embodiments, detection of nucleotides that encode DCN, nucleotides that encode a characteristic fragment of DCN, and/or nucleotides that encode variants of DCN is used in a method for assessing a subject's risk of developing TTR-CM, diagnosing a subject with TTR-CM or recommending a subject for additional cardiomyopathy testing. In some embodiments, nucleotides that encode DCN are detected in a sample with anti-DCN nucleotide sequence agents (e.g., anti-DCN nucleotide sequence antibody agents, probes, complementary nucleic acids, etc.).

### SPARC-Related Modular Calcium-Binding 2 (SMOC-2)

SPARC-related, modular calcium-binding protein 2 (SMOC-2), previously called SMAP2 (smooth muscle-associated protein 2), is a 55 kDa glycoprotein that is a member of the SPARC family of matricellular proteins. SMOC-2 promotes cell cycle progression by signaling through the integrin-linked kinase (ILK) to upregulate cyclin-D1. When expressed in the endothelial extracellular matrix, it potentiates growth factor-induced angiogenesis. SMOC-2 expression is upregulated during neointima formation, promoting proliferation and migration of vascular smooth muscle. In the skin, it promotes keratinocyte attachment and migration. SMOC-2 may also inhibit proteases in the lung and artery. SMOC-2 has been proposed as a biomarker for a number of cancers.

In some embodiments, SPARC-related modular calcium-binding 2 (SMOC2) is an ATTR Biomarker. In some embodiments, detection of SMOC2, a characteristic fragment of SMOC2, and/or a variant of SMOC2 is used in a method for assessing a subject's risk of developing TTR-CM, diagnosing a subject with TTR-CM or recommending a subject for additional cardiomyopathy testing. In some embodiments, SMOC2 is detected in a sample with anti-SMOC2 agents (e.g., anti-SMOC2 antibody agents, probes, etc.). In some embodiments, detection of nucleotides that encode SMOC2 nucleotides that encode a characteristic fragment of SMOC2, and/or nucleotides that encode variants of SMOC2 is used in a method for assessing a subject's risk of developing TTR-CM, diagnosing a subject with TTR-CM or recommending a subject for additional cardiomyopathy testing. In some embodiments, nucleotides that encode SMOC2 are detected in a sample with anti-SMOC2 nucleotide sequence agents (e.g., anti-SMOC2 nucleotide sequence antibody agents, probes, complementary nucleic acids, etc.).

Exemplary amino acid sequences for certain ATTR Biomarkers disclosed herein are included in Table 1 below.

TABLE 1
Exemplary ATTR Biomarker Amino Acid Sequences

| ATTR | UniProt | |
|---|---|---|
| Biomarker | ID | Exemplary Amino Acid Sequence |
| TnI | P19429 | MADGSSDAAREPRPAPAPIRRRSSNYRAYATEPHAKKKSKISAS |
| | | R |
| | | KLQLKTLLLQIAKQELEREAEERRGEKGRALSTRCQPLELAGLG |
| | | F |
| | | AELQDLCRQLHARVDKVDEERYDIEAKVTKNITEIADLTQKIFD |
| | | L |
| | | RGKFKRPTLRRVRISADAMMQALLGARAKESLDLRAHLKQVKKE |
| | | D TEKENREVGDWRKNIDALSGMEGRKKKFES (SEQ ID NO: |
| | | 1) |
| PKM1 | P14618-2 | MSKPHSEAGTAFIQTQQLHAAMADTFLEHMCRLDIDSPPITARN |
| | | T |
| | | GIICTIGPASRSVETLKEMIKSGMNVARLNFSHGTHEYHAETIK |
| | | N |
| | | VRTATESFASDPILYRPVAVALDTKGPEIRTGLIKGSGTAEVEL |
| | | K |
| | | KGATLKITLDNAYMEKCDENILWLDYKNICKVVEVGSKIYVDDG |
| | | L |
| | | ISLQVKQKGADFLVTEVENGGSLGSKKGVNLPGAAVDLPAVSEK |
| | | D |
| | | IQDLKFGVEQDVDMVFASFIRKASDVHEVRKVLGEKGKNIKIIS |
| | | K |
| | | IENHEGVRREDEILEASDGIMVARGDLGIEIPAEKVFLAQKMMI |
| | | G |
| | | RCNRAGKPVICATQMLESMIKKPRPTRAEGSDVANAVLDGADCI |
| | | M |
| | | LSGETAKGDYPLEAVRMOHLIAREAEAAMFHRKLFEELVRASSH |
| | | S |
| | | TDLMEAMAMGSVEASYKCLAAALIVLTESGRSAHQVARYRPRAP |
| | | I |
| | | IAVTRNPQTARQAHLYRGIFPVLCKDPVQEAWAEDVDLRVNFAM |
| | | N VGKARGFFKKGDVVIVLTGWRPGSGFTNTMRVVPVP (SEQ |
| | | ID NO: 2) |
| PKM2 | P14618-1 | MSKPHSEAGTAFIQTQQLHAAMADTFLEHMCRLDIDSPPITARN |
| | | T |
| | | GIICTIGPASRSVETLKEMIKSGMNVARLNFSHGTHEYHAETIK |
| | | N |
| | | VRTATESFASDPILYRPVAVALDTKGPEIRTGLIKGSGTAEVEL |
| | | K |
| | | KGATLKITLDNAYMEKCDENILWLDYKNICKVVEVGSKIYVDDG |
| | | L |
| | | ISLQVKQKGADFLVTEVENGGSLGSKKGVNLPGAAVDLPAVSEK |
| | | D |
| | | IQDLKFGVEQDVDMVFASFIRKASDVHEVRKVLGEKGKNIKIIS |
| | | K |
| | | IENHEGVRRFDEILEASDGIMVARGDLGIEIPAEKVFLAQKMMI |
| | | G |
| | | RCNRAGKPVICATQMLESMIKKPRPTRAEGSDVANAVLDGADCI |
| | | M |
| | | LSGETAKGDYPLEAVRMOHLIAREAEAAIYHLQLFEELRRLAPI |
| | | T |
| | | SDPTEATAVGAVEASFKCCSGAIIVLTKSGRSAHQVARYRPRAP |
| | | I |
| | | IAVTRNPQTARQAHLYRGIFPVLCKDPVQEAWAEDVDLRVNFAM |
| | | N VGKARGFFKKGDVVIVLTGWRPGSGFTNTMRVVPVP (SEQ |
| | | ID NO: 3) |
| NT- | P16860 | MDPQTAPSRALLLLLFLHLAFLGGRSHPLGSPGSASDLETSGLQ |
| proBNP | | E |
| | | QRNHLQGKLSELQVEQTSLEPLQESPRPTGVWKSREVATEGIRG |
| | | H |
| | | RKMVLYTLRAPRSPKMVQGSGCFGRKMDRISSSSGLGCKVLRRH |
| | | (SEQ ID NO: 4) |
| RBP4 | P02753 | MKWVWALLLLAALGSGRAERDCRVSSERVKENEDKARFSGTWYA |
| | | MA |
| | | KKDPEGLFLQDNIVAEFSVDETGQMSATAKGRVRLLNNWDVCAD |
| | | MV |
| | | GTFTDTEDPAKFKMKYWGVASFLQKGNDDHWIVDTDYDTYAVQY |
| | | SC |
| | | RLLNLDGTCADSYS FVFSRDPNGLPPEAQKIVRQRQEELCLARQ |
| | | YR LIVHNGYCDGRSERNLL (SEQ ID NO: 5) |
| DCN | P07585 | MKATI ILLLLAQVSWAGPFQQRGLEDEMLEDEASGIGPEVPDDR |
| | | DF |
| | | EPSLGPVCPFRCQCHLRVVQCSDLGLDKVPKDLPPDTTLLDLQN |
| | | NK |
| | | ITEIKDGDFKNLKNLHALILVNNKISKVSPGAFTPLVKLERLYL |
| | | SK |
| | | NQLKELPEKMPKTLQELRAHENEITKVRKVTENGLNQMIVIELG |
| | | TN |
| | | PLKSSGIENGAFQGMKKLSYIRIADTNITSIPQGLPPSLTELHL |
| | | DG |
| | | NKISRVDAASLKGLNNLAKLGLSFNSISAVDNGSLANTPHLREL |
| | | HL |
| | | DNNKLTRVPGGLAEHKYIQVVYLHNNNISVVGSSDFCPPGHNTK |
| | | KASYSGVSLESNPVQYWEIQPSTFRCVYVRSAIQLGNYK |
| | | (SEQ ID NO: 6) |
| TIMP2 | P16035 | MGAAARTLRLALGLLLLATLLRPADACSCSPVHPQQAFCNADVV |
| | | IR |
| | | AKAVSEKEVDSGNDIYGNPIKRIQYEIKQIKMFKGPEKDIEFIY |
| | | TA |
| | | PSSAVCGVSLDVGGKKEYLIAGKAEGDGKMHITLCDFIVPWDTL |
| | | ST |
| | | TOKKSLNHRYQMGCECKITRCPMIPCYISSPDECLWMDWVTEKN |
| | | IN GHQAKFFACIKRSDGSCAWYRGAAPPKQEFLDIEDP (SEQ |
| | | ID NO: 7) |
| SMOC-2 | Q9H3U7 | MLLPQLCWLPLLAGLLPPVPAQKFSALTFLRVDQDKDKDCSLDC |
| | | AG |
| | | SPQKPLCASDGRTFLSRCEFQRAKCKDPQLEIAYRGNCKDVSRC |
| | | VA |
| | | ERKYTQEQARKEFQQVFIPECNDDGTYSQVQCHSYTGYCWCVTP |
| | | NG |
| | | RPISGTAVAHKTPRCPGSVNEKLPQREGTGKTDDAAAPALETQP |
| | | QG |
| | | DEEDIASRYPTLWTEQVKSRONKTNKNSVSSCDQEHQSALEEAK |
| | | QP |
| | | KNDNVVIPECAHGGLYKPVQCHPSTGYCWCVLVDTGRPIPGTST |
| | | RY |
| | | EQPKCDNTARAHPAKARDLYKGRQLQGCPGAKKHEFLTSVLDAL |
| | | ST |
| | | DMVHAASDPSSSSGRLSEPDPSHTLEERVVHWYFKLLDKNSSGD |
| | | IG |
| | | KKEIKPFKRFLRKKSKPKKCVKKFVEYCDVNNDKSISVQELMGC |
| | | LG VAKEDGKADTKKRHTPRGHAESTSNRQPRKQG (SEQ ID |
| | | NO: 8) |
| NfL | P07196 | MSSFSYEPYYSTSYKRRYVETPRVHISSVRSGYSTARSAYSSYS |
| | | AP |
| | | VSSSLSVRRSYSSSSGSLMPSLENLDLSQVAAISNDLKSIRTQE |
| | | KA |
| | | QLQDLNDRFASFIERVHELEQQNKVLEAELLVLRQKHSEPSRER |
| | | AL |
| | | YEQEIRDLRLAAEDATNEKQALQGEREGLEETLRNLQARYEEEV |
| | | LS |
| | | REDAEGRLMEARKGADEAALARAELEKRIDSLMDEISFLKKVHE |
| | | EE |
| | | IAELQAQIQYAQISVEMDVTKPDLSAALKDIRAQYEKLAAKNMQ |
| | | NA |
| | | EEWFKSRFTVLTESAAKNTDAVRAAKDEVSESRRLLKAKTLEIE |
| | | AC |
| | | RGMNEALEKQLQELEDKQNADISAMQDTINKLENELRTTKSEMA |
| | | RY |
| | | LKEYQDLLNVKMALDIEIAAYRKLLEGEETRLSFTSVGSITSGY |
| | | SQ |
| | | SSQVFGRSAYGGLQTSSYLMSTRSFPSYYTSHVQEEQIEVEETI |
| | | EA |
| | | AKAEEAKDEPPSEGEAEEEEKDKEEAEEEEAAEEEEAAKEESEE |
| | | AKEEEEGGEGEEGEETKEAEEEEKKVEGAGEEQAAKKKD |
| | | (SEQ ID NO: 9) |

In some embodiments, additional markers may be analyzed or assessed. In some such embodiments, the additional markers include misfolded or aggregate transthyretin (TTR).

In some embodiments, additional factors are considered, including but not limited to demographic factors (e.g., one or more of age, weight, biological sex, ethnicity, BMI, medical history, risk factors, family history, and geographic location) and/or imaging-based biomarkers (e.g., left ventricle septal wall thickness and/or ejection fraction) of a subject from which a sample was obtained.

### Exemplary ATTR Biomarker Combinations

As provided herein, each ATTR Biomarker may be a full-length protein or a fragment thereof. In some embodiments, a fragment of an ATTR Biomarker is a characteristic fragment. In some embodiments, the ATTR Biomarkers are full length ATTR Biomarker proteins. In some embodiments, the ATTR Biomarkers are characteristic fragments of ATTR Biomarkers. In some embodiments, a subset of the ATTR Biomarkers are full length ATTR Biomarker proteins and a subset of the ATTR Biomarkers are characteristic fragments of ATTR Biomarkers.

In some embodiments, an ATTR Biomarker has a wild-type amino acid sequence. In some embodiments, an ATTR Biomarker has a variant amino acid sequence, e.g., an amino acid sequence including one or more mutations. In some embodiments, the ATTR Biomarkers each have a wild-type amino acid sequence. In some embodiments, the ATTR Biomarkers each have a variant amino acid sequence. In some embodiments, a subset of the ATTR Biomarkers each have a wild-type amino acid sequence and a subset of the ATTR Biomarkers each have a variant amino acid sequence.

In some embodiments, a combination (e.g., one or more, two or more, three or more, four or more, etc.) of ATTR Biomarkers includes a combination of ATTR Biomarkers listed below in Tables 2-5. In some embodiments, a combination (e.g., one or more, two or more, three or more, four or more, etc.) of ATTR Biomarkers includes a combination of ATTR Biomarkers listed below in Table 2. In some embodiments, a combination (e.g., one or more, two or more, three or more, four or more, etc.) of ATTR Biomarkers includes a combination of ATTR Biomarkers listed below in Table 3. In some embodiments, a combination (e.g., one or more, two or more, three or more, four or more, etc.) of ATTR Biomarkers includes a combination of ATTR Biomarkers listed below in Table 4. In some embodiments, a combination (e.g., one or more, two or more, three or more, four or more, etc.) of ATTR Biomarkers includes a combination of ATTR Biomarkers listed below in Table 5.

In some embodiments, a combination of ATTR Biomarkers includes one or more of TnI, PKM1, PKM2, NT-proBNP, RBP4, DCN, TIMP2, SMOC-2, NfL, or combinations thereof. In some embodiments, a combination of ATTR Biomarkers includes two or more of TnI, PKM1, PKM2, NT-proBNP, RBP4, DCN, TIMP2, SMOC-2, NfL, or combinations thereof. In some embodiments, a combination of ATTR Biomarkers includes three or more of TnI, PKM1, PKM2, NT-proBNP, RBP4, DCN, TIMP2, SMOC-2, NfL, or combinations thereof. In some embodiments, a combination of ATTR Biomarkers includes four or more of TnI, PKM1, PKM2, NT-proBNP, RBP4, DCN, TIMP2, SMOC-2, NfL, or combinations thereof. In some embodiments, a combination of ATTR Biomarkers includes five or more of TnI, PKM1, PKM2, NT-proBNP, RBP4, DCN, TIMP2, SMOC-2, NfL, or combinations thereof. In some embodiments, a combination of ATTR Biomarkers includes six or more of TnI, PKM1, PKM2, NT-proBNP, RBP4, DCN, TIMP2, SMOC-2, NfL, or combinations thereof. In some embodiments, a combination of ATTR Biomarkers includes seven or more of TnI, PKM1, PKM2, NT-proBNP, RBP4, DCN, TIMP2, SMOC-2, NfL, or combinations thereof. In some embodiments, a combination of ATTR Biomarkers includes eight or more of TnI, PKM1, PKM2, NT-proBNP, RBP4, DCN, TIMP2, SMOC-2, NfL, or combinations thereof. In some embodiments, a combination of ATTR Biomarkers includes TnI, PKM1, PKM2, NT-proBNP, RBP4, DCN, TIMP2, SMOC-2, or NfL.

In some embodiments, a combination of ATTR Biomarkers includes TnI. In some embodiments, a combination of ATTR Biomarkers includes TnI and PKM1. In some embodiments, a combination of ATTR Biomarkers includes TnI and PKM2. In some embodiments, a combination of ATTR Biomarkers includes TnI, PKM1, and PKM2. In some embodiments, a combination of ATTR Biomarkers further includes NT-proBNP, RBP4, or both. In some embodiments, a combination of ATTR Biomarkers further includes TIMP2, NfL, or both.

In some embodiments, a combination of ATTR Biomarkers includes NT-proBNP. In some embodiments, a combination of ATTR Biomarkers further includes TnI, PKM1, PKM2, RBP4, or a combination thereof. In some embodiments, a combination of ATTR Biomarkers further includes TIMP2, NfL, or both.

In some embodiments, a combination (e.g., one or more, two or more, three or more, four or more, etc.) of ATTR Biomarkers includes TnI, PKM2, NT-proBNP, and RBP4. In some embodiments, a combination (e.g., one or more, two or more, three or more, four or more, etc.) of ATTR Biomarkers includes TnI, PKM2, and NT-proBNP. In some embodiments, a combination (e.g., one or more, two or more, three or more, four or more, etc.) of ATTR Biomarkers includes TnI, PKM2, and RBP4. In some embodiments, a combination (e.g., one or more, two or more, three or more, four or more, etc.) of ATTR Biomarkers includes TnI, PKM1, and NT-proBNP. In some embodiments, a combination (e.g., one or more, two or more, three or more, four or more, etc.) of ATTR Biomarkers includes TnI, PKM1, and RBP4. In some embodiments, a combination (e.g., one or more, two or more, three or more, four or more, etc.) of ATTR Biomarkers includes TnI, PKM1, PKM2, NT-proBNP, and RBP4. In some embodiments, a combination (e.g., one or more, two or more, three or more, four or more, etc.) of ATTR Biomarkers includes TnI, PKM1, PKM2, and NT-proBNP. In some embodiments, a combination (e.g., one or more, two or more, three or more, four or more, etc.) of ATTR Biomarkers includes TnI, PKM1, PKM2, and RBP4. In some embodiments, a combination (e.g., one or more, two or more, three or more, four or more, etc.) of ATTR Biomarkers includes NT-proBNP, RBP4, and TnI. In some embodiments, a combination (e.g., one or more, two or more, three or more, four or more, etc.) of ATTR Biomarkers includes RBP4, SMOC-2, and TnI. In some embodiments, a combination (e.g., one or more, two or more, three or more, four or more, etc.) of ATTR Biomarkers includes DCN, NT-proBNP, and TnI. In some embodiments, a combination (e.g., one or more, two or more, three or more, four or more, etc.) of ATTR Biomarkers includes DCN, RBP4, and TnI. In some embodiments, a combination (e.g., one or more, two or more, three or more, four or more, etc.) of ATTR Biomarkers includes NT-proBNP, SMOC-2, and TnI. In some embodiments, a combination (e.g., one or more, two or more, three or more, four or more, etc.) of ATTR Biomarkers includes NT-proBNP, TIMP2, and TnI. In some embodiments, a combination (e.g., one or more, two or more, three or more, four or more, etc.) of ATTR Biomarkers includes NT-proBNP and TnI. In some embodiments, a combination (e.g., one or more, two or more, three or more, four or more, etc.) of ATTR Biomarkers includes DCN and TnI. In some embodiments, a combination (e.g., one or more, two or more, three or more, four or more, etc.) of ATTR Biomarkers includes DCN, TIMP2, and TnI. In some embodiments, a combination (e.g., one or more, two or more, three or more, four or more, etc.) of ATTR Biomarkers includes RBP4 and TnI. In some embodiments, a combination (e.g., one or more, two or more, three or more, four or more, etc.) of ATTR Biomarkers includes RBP4, TIMP2, and TnI. In some embodiments, a combination (e.g., one or more, two or more, three or more, four or more, etc.) of ATTR Biomarkers includes DCN, SMOC-2, and TnI. In some embodiments, a combination (e.g., one or more, two or more, three or more, four or more, etc.) of ATTR Biomarkers includes TIMP2 and TnI. In some embodiments, a combination (e.g., one or more, two or more, three or more, four or more, etc.) of ATTR Biomarkers includes SMOC-2, TIMP2, and TnI.

Exemplary combinations of ATTR Biomarkers consistent with the present disclosure are included in Table 2 below. Also disclosed herein are combinations of ATTR Biomarkers including the combinations listed Table 2 below.

TABLE 2
Exemplary Combinations of ATTR Biomarkers
Exemplary ATTR Biomarker Combinations

TnI, PKM2, NT-proBNP, RBP4
TnI, PKM2, NT-proBNP
TnI, PKM2, RBP4
TnI, PKM1, NT-proBNP, RBP4
TnI, PKM1, NT-proBNP
TnI, PKM1, RBP4
TnI, PKM1, PKM2, NT-proBNP, RBP4
TnI, PKM1, PKM2, NT-proBNP
TnI, PKM1, PKM2, RBP4
NT-proBNP, RBP4, TnI
RBP4, SMOC-2, TnI
DCN, NT-proBNP, TnI
DCN, RBP4, TnI
NT-proBNP, SMOC-2, TnI
NT-proBNP, TIMP2, TnI
NT-proBNP, TnI
DCN, TnI
DCN, TIMP2, TnI
RBP4, TnI
RBP4, TIMP2, TnI
DCN, SMOC-2, TnI
TIMP2, TnI
SMOC-2, TIMP2, TnI

Exemplary combinations including two ATTR Biomarkers consistent with present disclosure are included in Table 3 below. Also disclosed herein are combinations ATTR Biomarkers including the combinations listed Table 3 below.

TABLE 3
Exemplary Combinations Including Two ATTR Biomarkers
Exemplary Two ATTR Biomarker Combinations

TnI, PKM1
TnI, PKM2
TnI, NT-proBNP
TnI, RBP4
TnI, DCN
TnI, TIMP2
TnI, SMOC-2
TnI, NfL
PKM1, NT-proBNP
PKM1, RBP4
PKM1, DCN
PKM1, TIMP2
PKM1, SMOC-2
PKM1, NfL
PKM2, NT-proBNP
PKM2, RBP4
PKM2, DCN
PKM2, TIMP2
PKM2, SMOC-2
PKM2, NfL
NT-proBNP, RBP4
NT-proBNP, DCN
NT-proBNP, TIMP2
NT-proBNP, SMOC-2
NT-proBNP, NfL
RBP4, DCN
RBP4, TIMP2
RBP4, SMOC-2
RBP4, NfL
DCN, TIMP2
DCN, SMOC-2
DCN, NfL
TIMP2, SMOC-2
TIMP2, NfL
SMOC-2, NfL

Exemplary combinations including three ATTR Biomarkers consistent with the present disclosure are included in Table 4 below. Also disclosed herein are combination of ATTR Biomarkers including the combinations listed Table 4 below.

TABLE 4
Exemplary Combinations Including Three ATTR Biomarkers
Exemplary Three ATTR Biomarker Combinations

TnI, PKM1, PKM2
TnI, PKM1, NT-proBNP
TnI, PKM1, RBP4
TnI, PKM1, DCN
TnI, PKM1, TIMP2
TnI, PKM1, SMOC-2
TnI, PKM1, NfL
TnI, PKM2, NT-proBNP
TnI, PKM2, RBP4
TnI, PKM2, DCN
TnI, PKM2, TIMP2
TnI, PKM2, SMOC-2
TnI, PKM2, NfL
TnI, NT-proBNP, RBP4
TnI, NT-proBNP, DCN
TnI, NT-proBNP, TIMP2
TnI, NT-proBNP, SMOC-2
TnI, NT-proBNP, NfL
TnI, RBP4, DCN
TnI, RBP4, TIMP2
TnI, RBP4, SMOC-2
TnI, RBP4, NfL
TnI, DCN, TIMP2
TnI, DCN, SMOC-2
TnI, DCN, NfL
TnI, TIMP2, SMOC-2
TnI, TIMP2, NfL
TnI, SMOC-2, NfL
PKM1, PKM2, NT-proBNP
PKM1, PKM2, RBP4
PKM1, PKM2, DCN
PKM1, PKM2, TIMP2
PKM1, PKM2, SMOC-2
PKM1, PKM2, NfL
PKM1, NT-proBNP, RBP4
PKM1, NT-proBNP, DCN
PKM1, NT-proBNP, TIMP2
PKM1, NT-proBNP, SMOC-2
PKM1, NT-proBNP, NfL
PKM1, RBP4, DCN
PKM1, RBP4, TIMP2
PKM1, RBP4, SMOC-2
PKM1, RBP4, NfL
PKM1, DCN, TIMP2
PKM1, DCN, SMOC-2
PKM1, DCN, NfL
PKM1, TIMP2, SMOC-2
PKM1, TIMP2, NfL
PKM1, SMOC-2, NfL
PKM2, NT-proBNP, RBP4
PKM2, NT-proBNP, DCN
PKM2, NT-proBNP, TIMP2
PKM2, NT-proBNP, SMOC-2
PKM2, NT-proBNP, NfL
PKM2, RBP4, DCN
PKM2, RBP4, TIMP2
PKM2, RBP4, SMOC-2
PKM2, RBP4, NfL
PKM2, DCN, TIMP2
PKM2, DCN, SMOC-2
PKM2, DCN, NfL
PKM2, TIMP2, SMOC-2
PKM2, TIMP2, NfL
PKM2, SMOC-2, NfL
NT-proBNP, RBP4, DCN
NT-proBNP, RBP4, TIMP2
NT-proBNP, RBP4, SMOC-2
NT-proBNP, RBP4, NfL
NT-proBNP, DCN, TIMP2
NT-proBNP, DCN, SMOC-2
NT-proBNP, DCN, NfL
NT-proBNP, TIMP2, SMOC-2
NT-proBNP, TIMP2, NfL
NT-proBNP, SMOC-2, NfL
RBP4, DCN, TIMP2
RBP4, DCN, SMOC-2
RBP4, DCN, NfL
RBP4, TIMP2, SMOC-2
RBP4, TIMP2, NfL
RBP4, SMOC-2, NfL
DCN, TIMP2, SMOC-2
DCN, TIMP2, NfL
DCN, SMOC-2, NfL
TIMP2, SMOC-2, NfL

Exemplary combinations including four ATTR Biomarkers consistent with the present disclosure are included in Table 5 below. Also disclosed herein are combinations of ATTR Biomarkers including the combinations listed Table 5 below.

TABLE 5
Exemplary Combinations Including Four ATTR Biomarkers
Exemplary Four ATTR Biomarker Combinations

TnI, PKM1, PKM2, NT-proBNP
TnI, PKM1, PKM2, RBP4
TnI, PKM1, PKM2, DCN
TnI, PKM1, PKM2, TIMP2
TnI, PKM1, PKM2, SMOC-2
TnI, PKM1, PKM2, NfL
TnI, PKM1, NT-proBNP, RBP4
TnI, PKM1, NT-proBNP, DCN
TnI, PKM1, NT-proBNP, TIMP2
TnI, PKM1, NT-proBNP, SMOC-2
TnI, PKM1, NT-proBNP, NfL
TnI, PKM1, RBP4, DCN
TnI, PKM1, RBP4, TIMP2
TnI, PKM1, RBP4, SMOC-2
TnI, PKM1, RBP4, NfL
TnI, PKM1, DCN, TIMP2
TnI, PKM1, DCN, SMOC-2
TnI, PKM1, DCN, NfL
TnI, PKM1, TIMP2, SMOC-2
TnI, PKM1, TIMP2, NfL
TnI, PKM1, SMOC-2, NfL
TnI, PKM2, NT-proBNP, RBP4
TnI, PKM2, NT-proBNP, DCN
TnI, PKM2, NT-proBNP, TIMP2
TnI, PKM2, NT-proBNP, SMOC-2
TnI, PKM2, NT-proBNP, NfL
TnI, PKM2, RBP4, DCN
TnI, PKM2, RBP4, TIMP2
TnI, PKM2, RBP4, SMOC-2
TnI, PKM2, RBP4, NfL
TnI, PKM2, DCN, TIMP2
TnI, PKM2, DCN, SMOC-2
TnI, PKM2, DCN, NfL
TnI, PKM2, TIMP2, SMOC-2
TnI, PKM2, TIMP2, NfL
TnI, PKM2, SMOC-2, NfL
TnI, NT-proBNP, RBP4, DCN
TnI, NT-proBNP, RBP4, TIMP2
TnI, NT-proBNP, RBP4, SMOC-2
TnI, NT-proBNP, RBP4, NfL
TnI, NT-proBNP, DCN, TIMP2
TnI, NT-proBNP, DCN, SMOC-2
TnI, NT-proBNP, DCN, NfL
TnI, NT-proBNP, TIMP2, SMOC-2
TnI, NT-proBNP, TIMP2, NfL
TnI, NT-proBNP, SMOC-2, NfL
TnI, RBP4, DCN, TIMP2
TnI, RBP4, DCN, SMOC-2
TnI, RBP4, DCN, NfL
TnI, RBP4, TIMP2, SMOC-2
TnI, RBP4, TIMP2, NfL
TnI, RBP4, SMOC-2, NfL
TnI, DCN, TIMP2, SMOC-2
TnI, DCN, TIMP2, NfL
TnI, DCN, SMOC-2, NfL
TnI, TIMP2, SMOC-2, NfL
PKM1, PKM2, NT-proBNP, RBP4
PKM1, PKM2, NT-proBNP, DCN
PKM1, PKM2, NT-proBNP, TIMP2
PKM1, PKM2, NT-proBNP, SMOC-2
PKM1, PKM2, NT-proBNP, NfL
PKM1, PKM2, RBP4, DCN
PKM1, PKM2, RBP4, TIMP2
PKM1, PKM2, RBP4, SMOC-2
PKM1, PKM2, RBP4, NfL
PKM1, PKM2, DCN, TIMP2
PKM1, PKM2, DCN, SMOC-2
PKM1, PKM2, DCN, NfL
PKM1, PKM2, TIMP2, SMOC-2
PKM1, PKM2, TIMP2, NfL
PKM1, PKM2, SMOC-2, NfL
PKM1, NT-proBNP, RBP4, DCN
PKM1, NT-proBNP, RBP4, TIMP2
PKM1, NT-proBNP, RBP4, SMOC-2
PKM1, NT-proBNP, RBP4, NfL
PKMI, NT-proBNP, DCN, TIMP2
PKM1, NT-proBNP, DCN, SMOC-2
PKM1, NT-proBNP, DCN, NfL
PKM1, NT-proBNP, TIMP2, SMOC-2
PKM1, NT-proBNP, TIMP2, NfL
PKMI, NT-proBNP, SMOC-2, NfL
PKM1, RBP4, DCN, TIMP2
PKM1, RBP4, DCN, SMOC-2
PKM1, RBP4, DCN, NfL
PKM1, RBP4, TIMP2, SMOC-2
PKM1, RBP4, TIMP2, NfL
PKM1, RBP4, SMOC-2, NfL
PKM1, DCN, TIMP2, SMOC-2
PKM1, DCN, TIMP2, NfL
PKM1, DCN, SMOC-2, NfL
PKM1, TIMP2, SMOC-2, NfL
PKM2, NT-proBNP, RBP4, DCN
PKM2, NT-proBNP, RBP4, TIMP2
PKM2, NT-proBNP, RBP4, SMOC-2
PKM2, NT-proBNP, RBP4, NfL
PKM2, NT-proBNP, DCN, TIMP2
PKM2, NT-proBNP, DCN, SMOC-2
PKM2, NT-proBNP, DCN, NIL
PKM2, NT-proBNP, TIMP2, SMOC-2
PKM2, NT-proBNP, TIMP2, NfL
PKM2, NT-proBNP, SMOC-2, NfL
PKM2, RBP4, DCN, TIMP2
PKM2, RBP4, DCN, SMOC-2
PKM2, RBP4, DCN, NfL
PKM2, RBP4, TIMP2, SMOC-2
PKM2, RBP4, TIMP2, NfL
PKM2, RBP4, SMOC-2, NfL
PKM2, DCN, TIMP2, SMOC-2
PKM2, DCN, TIMP2, NfL
PKM2, DCN, SMOC-2, NfL
PKM2, TIMP2, SMOC-2, NfL
NT-proBNP, RBP4, DCN, TIMP2
NT-proBNP, RBP4, DCN, SMOC-2
NT-proBNP, RBP4, DCN, NfL
NT-proBNP, RBP4, TIMP2, SMOC-2
NT-proBNP, RBP4, TIMP2, NfL
NT-proBNP, RBP4, SMOC-2, NfL
NT-proBNP, DCN, TIMP2, SMOC-2
NT-proBNP, DCN, TIMP2, NfL
NT-proBNP, DCN, SMOC-2, NfL
NT-proBNP, TIMP2, SMOC-2, NfL
RBP4, DCN, TIMP2, SMOC-2
RBP4, DCN, TIMP2, NfL
RBP4, DCN, SMOC-2, NfL
RBP4, TIMP2, SMOC-2, NfL
DCN, TIMP2, SMOC-2, NfL

In some embodiments, a combination (e.g., one or more, two or more, three or more, four or more, etc.) of ATTR Biomarkers does not include PKM1. In some embodiments, a combination (e.g., one or more, two or more, three or more, four or more, etc.) of ATTR Biomarkers does not include PKM2. In some embodiments, a combination (e.g., one or more, two or more, three or more, four or more, etc.) of ATTR Biomarkers does not include either PKM1 or PKM2.

In some embodiments, a combination (e.g., one or more, two or more, three or more, four or more, etc.) of ATTR Biomarkers does not include SMOC-2. In some embodiments, a combination (e.g., one or more, two or more, three or more, four or more, etc.) of ATTR Biomarkers does not include DCN. In some embodiments, a combination (e.g., one or more, two or more, three or more, four or more, etc.) of ATTR Biomarkers does not include either SMOC-2 or DCN.

### Methods

Methods of the present disclosure further allow for earlier identification of more patients who are at risk of TTR-CM, while minimizing the number of false negatives. In some embodiments, methods disclosed herein provide the advantage of an early screen for the presence of ATTRwt. In some embodiments, methods disclosed herein can assist in the detection or diagnosis of ATTRwt after genetic testing rules out ATTRm. In some embodiments, methods disclosed herein reduces or eliminates the need for initiating screening for TTR-CM with costly and complex process of an echocardiogram, followed by CMR and scintigraphy. In some embodiments, if methods disclosed herein result in detection of possible TTR-CM in a subject, the subject can undergo subsequent confirmatory testing, such as echocardiogram, cardiac magnetic resonance imaging (CMR), scintigraphy, and/or cardiac biopsy.

In some embodiments, a method disclosed herein is a method of determining a subject's risk of developing amyloid transthyretin cardiomyopathy (TTR-CM). In some embodiments, a method disclosed herein is a method of diagnosing a subject with TTR-CM, and the sample was obtained from the subject. In some embodiments, a method disclosed herein is a method of treating TTR-CM in a subject at risk of or suffering from TTR-CM. In some embodiments, a method disclosed herein is a method of determining a patient does not have or is not at risk of developing TTR-CM.

In some embodiments, TTR-CM results from wild-type transthyretin amyloidosis (ATTRwt). In some embodiments, a subject tests negative for familial amyloid cardiomyopathy (ATTRm) by genetic testing.

In some embodiments, a method disclosed herein is a method of selecting a subject to receive one or more doses of a TTR stabilizing agent, and the sample was obtained from the subject. In some embodiments, a method disclosed herein includes administering to the subject one or more doses a TTR stabilizing agent.

In some embodiments, a method disclosed herein is a method of selecting a subject for one or more cardiomyopathy tests, and the sample was obtained from the subject. In some embodiments, one or more cardiomyopathy tests include an echocardiogram, an advanced imaging method, or both. In some embodiments, an advanced imaging method includes cardiac magnetic resonance imaging (CMR), scintigraphy, or both. In some embodiments, scintigraphy includes use of a radioisotope conjugate such as 99m Tc-Pyrophosphate. In some embodiments, scintigraphy is performed using single photon emission computed tomography (SPECT).

The present disclosure provides diagnostic tests for TTR-CM (including TTR-CM resulting from wild-type ATTR amyloidosis) characterized by detection of biomarkers according to the methods above.

In some embodiments, methods of detecting, diagnosing, or identifying a risk of TTR-CM as taught by the present disclosure are improved methods as compared to standard techniques in that the methods of the present disclosure include one or more of the following benefits: improved sensitivity for identifying TTR-CM, improved specificity for identifying TTR-CM, improved accuracy for identifying TTR-CM, reduced time to diagnosis for TTR-CM, and/or reduced cost of screening patients for TTR-CM.

In some embodiments, the biomarkers disclosed herein can be used for an in-vitro diagnostic (IVD) or screening test for the condition of amyloid transthyretin cardiomyopathy. In some embodiments, a diagnostic test as taught by the present disclosure detects whether one or more biomarkers are present in a sample obtained from a subject. In some embodiments, a diagnostic test as taught by the present disclosure can assist in the detection or diagnosis of TTR-CM (including TTR-CM resulting from wild-type ATTR amyloidosis) in a subject.

In some embodiments, a diagnostic test as taught by the present disclosure is adapted to an immunoassay platform. In some embodiments, such an immunoassay platform includes a semi-automated or automated immunoassay platform. In some embodiments, a diagnostic test as taught by the present disclosure is adapted for semi-automated testing of one or more biomarkers.

In some embodiments, a diagnostic test as taught by the present disclosure is an improved diagnostic for TTR-CM as compared to standard techniques in that the diagnostic test of the present disclosure includes one or more of the following benefits: improved sensitivity for identifying TTR-CM, improved specificity for identifying TTR-CM, improved accuracy for identifying TTR-CM, reduced time to diagnosis for TTR-CM, and/or reduced cost of screening patients for TTR-CM.

In some embodiments, a diagnostic test as disclosed herein can be a plasma-based screening assay. In some embodiments, a diagnostic test is adapted for, e.g., the Siemens Atellica® system or the Siemens Advia Centaur® system.

As set out in greater detail below, in some embodiments, methods provided herein include detecting a level of each of two or more transthyretin amyloidosis (ATTR) Biomarkers are present in a sample.

In some embodiments, methods provided herein include detecting a level of each of two or more transthyretin amyloidosis (ATTR) Biomarkers in a sample to obtain an ATTR Biomarker profile, and using the ATTR Biomarker profile to compute an ATTR Biomarker score. In some embodiments, methods provided herein include detecting a level of each of two or more transthyretin amyloidosis (ATTR) Biomarkers in a sample to obtain an ATTR Biomarker profile, and using the ATTR Biomarker profile and demographic factors to compute an ATTR Biomarker score. In some embodiments, methods provided herein include detecting a level of each of two or more transthyretin amyloidosis (ATTR) Biomarkers in a sample to obtain an ATTR Biomarker profile, and using the ATTR Biomarker profile and imaging-based biomarkers to compute an ATTR Biomarker score. In some embodiments, methods provided herein include detecting a level of each of two or more transthyretin amyloidosis (ATTR) Biomarkers in a sample to obtain an ATTR Biomarker profile, and using the ATTR Biomarker profile, demographic factors, and imaging-based biomarkers to compute an ATTR Biomarker score.

In some embodiments, methods provided herein including receiving a level of each of two or more transthyretin amyloidosis (ATTR) Biomarkers in a sample. In some embodiments, receiving includes electronically receiving.

In some embodiments, methods provided herein include using a level of each of two or more transthyretin amyloidosis (ATTR) Biomarkers in a sample to obtain an ATTR Biomarker profile, and using the ATTR Biomarker profile to compute an ATTR Biomarker score. In some embodiments, methods provided herein include using a level of each of two or more transthyretin amyloidosis (ATTR) Biomarkers in a sample to obtain an ATTR Biomarker profile, and using the ATTR Biomarker profile and demographic factors to compute an ATTR Biomarker score. In some embodiments, methods provided herein include using a level of each of two or more transthyretin amyloidosis (ATTR) Biomarkers in a sample to obtain an ATTR Biomarker profile, and using the ATTR Biomarker profile and imaging-based biomarkers to compute an ATTR Biomarker score. In some embodiments, methods provided herein include using a level of each of two or more transthyretin amyloidosis (ATTR) Biomarkers in a sample to obtain an ATTR Biomarker profile, and using the ATTR Biomarker profile, demographic factors, and imaging-based biomarkers to compute an ATTR Biomarker score.

In some embodiments, demographic factors include one or more of age, weight, biological sex, ethnicity, BMI, medical history, risk factors, family history, and geographic location. In some embodiments, imaging-based biomarkers include left ventricle septal wall thickness and/or ejection fraction.

In some embodiments, methods described herein include using an ATTR Biomarker score to select a subject for further cardiomyopathy tests. In some embodiments, methods described herein include using an ATTR Biomarker score to select a subject to received one or more doses of a TTR stabilizing agent. In some embodiments, methods described herein include using an ATTR Biomarker score to identify a subject as having or being at risk of having TTR-CM.

In some embodiments, methods described herein include comparing an ATTR Biomarker score to a reference ATTR Biomarker score. In some embodiments, methods described herein include administering one or more doses of a TTR stabilizing agent to a subject. In some embodiments, methods described herein include performing one or more cardiomyopathy tests on a subject.

Additional description of exemplary methods disclosed herein are provided below.

### Detecting ATTR Biomarkers and Obtaining an ATTR Biomarker Profile

Among other things, methods provided herein include assessment of a level of one or more ATTR Biomarkers in a sample. A level of an ATTR Biomarker can be detected in a sample. Exemplary methods for detecting a level of one or more ATTR Biomarkers are described herein. However, a level of an ATTR Biomarker can also be provided, for example, in electronic form from, e.g., a laboratory that has detected a level of one or more ATTR Biomarkers in sample.

In some embodiments, the present disclosure provides technologies according to which one or more ATTR Biomarkers are detected, analyzed and/or assessed in a sample. In some embodiments, one or more ATTR Biomarkers are in a sample obtained from a subject; in some embodiments, a diagnosis or therapeutic decision is made based on such detection, analysis and/or assessment. In some embodiments, the biomarkers that are detected, analyzed or assessed are one or more ATTR Biomarkers (e.g., TnI, PKM1, PKM2, NT-proBNP, RBP4, DCN, TIMP2, SMOC-2, NfL, or combinations thereof).

In some embodiments, the present disclosure provides a method of detecting a level of one or more ATTR Biomarkers (e.g., TnI, PKM1, PKM2, NT-proBNP, RBP4, DCN, TIMP2, SMOC-2, NfL, or combinations thereof) in a sample. A level of an ATTR Biomarker, as described herein, encompasses the presence of an ATTR Biomarker, the absence of an ATTR Biomarker, an amount of an ATTR Biomarker, an absolute amount of an ATTR Biomarker, a relative amount of an ATTR Biomarker, or a concentration of an ATTR Biomarker.

In some embodiments, a method provided herein includes detecting a level of each ATTR Biomarker of a combination listed in Tables 2-5 in a sample. In some embodiments, a method provided herein includes detecting a level of each ATTR Biomarker of a combination listed in Table 2 in a sample. In some embodiments, a method provided herein includes detecting a level of each ATTR Biomarker of a combination listed in Table 3 in a sample. In some embodiments, a method provided herein includes detecting a level of each ATTR Biomarker of a combination listed in Table 4 in a sample. In some embodiments, a method provided herein includes detecting a level of each ATTR Biomarker of a combination listed in Table 5 in a sample.

In some embodiments, a method provided herein includes detecting a level of each of one or more ATTR Biomarkers in a sample, wherein the one or more ATTR Biomarkers include TnI, PKM1, PKM2, NT-proBNP, RBP4, DCN, TIMP2, SMOC-2, NfL, or combinations thereof. In some embodiments, a method provided herein includes detecting a level of each of two or more ATTR Biomarkers in a sample, wherein the two or more ATTR Biomarkers include TnI, PKM1, PKM2, NT-proBNP, RBP4, DCN, TIMP2, SMOC-2, NfL, or combinations thereof. In some embodiments, a method provided herein includes detecting a level of each of three or more ATTR Biomarkers in a sample, wherein the three or more ATTR Biomarkers include TnI, PKM1, PKM2, NT-proBNP, RBP4, DCN, TIMP2, SMOC-2, NfL, or combinations thereof. In some embodiments, a method provided herein includes detecting a level of each of four or more ATTR Biomarkers in a sample, wherein the four or more ATTR Biomarkers include TnI, PKM1, PKM2, NT-proBNP, RBP4, DCN, TIMP2, SMOC-2, NfL, or combinations thereof. In some embodiments, a method provided herein includes detecting a level of each of five or more ATTR Biomarkers in a sample, wherein the five or more ATTR Biomarkers include TnI, PKM1, PKM2, NT-proBNP, RBP4, DCN, TIMP2, SMOC-2, NfL, or combinations thereof. In some embodiments, a method provided herein includes detecting a level of each of six or more ATTR Biomarkers in a sample, wherein the six or more ATTR Biomarkers include TnI, PKM1, PKM2, NT-proBNP, RBP4, DCN, TIMP2, SMOC-2, NfL, or combinations thereof. In some embodiments, a method provided herein includes detecting a level of each of seven or more ATTR Biomarkers in a sample, wherein the seven or more ATTR Biomarkers include TnI, PKM1, PKM2, NT-proBNP, RBP4, DCN, TIMP2, SMOC-2, and NfL. In some embodiments, a method provided herein includes detecting a level of each of eight or more ATTR Biomarkers in a sample, wherein the eight or more ATTR Biomarkers include TnI, PKM1, PKM2, NT-proBNP, RBP4, DCN, TIMP2, SMOC-2, and NfL. In some embodiments, a method provided herein includes detecting a level of each of TnI, PKM1, PKM2, NT-proBNP, RBP4, DCN, TIMP2, SMOC-2, and NfL in a sample.

In some embodiments, a method provided herein includes detecting a level of TnI in a sample. In some embodiments, a method provided herein includes detecting a level of TnI and PKM1 in a sample. In some embodiments, a method provided herein includes detecting a level of TnI and PKM2 in a sample. In some embodiments, a method provided herein includes detecting a level of TnI, PKM1, and PKM2 in a sample. In some embodiments, a method provided herein further includes detecting a level of NT-proBNP, RBP4, or both in a sample. In some embodiments, a method provided herein further includes detecting a level of TIMP2, NfL, or both in a sample.

In some embodiments, a method provided herein includes detecting a level of NT-proBNP in a sample. In some embodiments, a method provided herein further includes detecting a level of TnI, PKM1, PKM2, RBP4, or a combination thereof in a sample. In some embodiments, a method provided herein further includes detecting a level of TIMP2, NfL, or both in a sample.

In some embodiments, a method provided herein includes detecting a level of each of TnI, PKM2, NT-proBNP, and RBP4 in a sample. In some embodiments, a method provided herein includes detecting a level of each of TnI, PKM2, and NT-proBNP in a sample. In some embodiments, a method provided herein includes detecting a level of each of TnI, PKM2, and RBP4 in a sample. In some embodiments, a method provided herein includes detecting a level of each of TnI, PKM1, and NT-proBNP in a sample. In some embodiments, a method provided herein includes detecting a level of each of TnI, PKM1, and RBP4 in a sample. In some embodiments, a method provided herein includes detecting a level of each of TnI, PKM1, PKM2, NT-proBNP, and RBP4 in a sample. In some embodiments, a method provided herein includes detecting a level of each of TnI, PKM1, PKM2, and NT-proBNP in a sample. In some embodiments, a method provided herein includes detecting a level of each of TnI, PKM1, PKM2, and RBP4 in a sample. In some embodiments, a method provided herein includes detecting a level of each of NT-proBNP, RBP4, and TnI in a sample. In some embodiments, a method provided herein includes detecting a level of each of RBP4, SMOC-2, and TnI in a sample. In some embodiments, a method provided herein includes detecting a level of each of DCN, NT-proBNP, and TnI in a sample. In some embodiments, a method provided herein includes detecting a level of each of DCN, RBP4, and TnI in a sample. In some embodiments, a method provided herein includes detecting a level of each of NT-proBNP, SMOC-2, and TnI in a sample. In some embodiments, a method provided herein includes detecting a level of each of NT-proBNP, TIMP2, and TnI in a sample. In some embodiments, a method provided herein includes detecting a level of each of NT-proBNP and TnI in a sample. In some embodiments, a method provided herein includes detecting a level of each of DCN and TnI in a sample. In some embodiments, a method provided herein includes detecting a level of each of DCN, TIMP2, and TnI in a sample. In some embodiments, a method provided herein includes detecting a level of each of RBP4 and TnI in a sample. In some embodiments, a method provided herein includes detecting a level of each of RBP4, TIMP2, and TnI in a sample. In some embodiments, a method provided herein includes detecting a level of each of DCN, SMOC-2, and TnI in a sample. In some embodiments, a method provided herein includes detecting a level of each of TIMP2 and TnI in a sample. In some embodiments, a method provided herein includes detecting a level of each of SMOC-2, TIMP2, and TnI in a sample.

Methods of detecting biomarkers (e.g., ATTR Biomarkers) include methods for detecting biomarkers as proteins. Protein-based methods of detecting biomarkers include, for example, mass spectrometry (MS), immunoassays (e.g., immunoprecipitation), Western blots, ELISAs, immunohistochemistry, immunocytochemistry, flow cytometry, and/or immuno-PCR.

In some embodiments, mass spectrometry includes MS, MS/MS, MALDI-TOF, electrospray ionization mass spectrometry (ESIMS), ESI-MS/MS, ESI-MS/(MS)ₙ, matrix-assisted laser desorption ionization time-of-flight mass spectrometry (MALDI-TOF-MS), surface-enhanced laser desorption/ionization time-of-flight mass spectrometry (SELDI-TOF-MS), tandem liquid chromatography-mass spectrometry (LC-MS/MS) mass spectrometry, desorption/ionization on silicon (DIOS), secondary ion mass spectrometry (SIMS), quadrupole time-of-flight (Q-TOF), atmospheric pressure chemical ionization mass spectrometry (APCI-MS), APCI-MS/MS, APCI-(MS), atmospheric pressure photoionization mass spectrometry (APPI-MS), APPI-MS/MS, and APPI-(MS)ₙ, quadrupole mass spectrometry, Fourier transform mass spectrometry (FTMS), and ion trap mass spectrometry. Often, the MS approach quantifies a fragment of a biomarker rather than the full-length protein. MS approaches, however, can be sufficient to determine the protein level of the biomarker to an accuracy sufficient for methods and/or assessments as disclosed.

In some embodiments, an immunoassay can be a chemiluminescent immunoassay. In some embodiments, an immunoassay can be a high-throughput and/or automated immunoassay platform. For example, a high-throughput and/or automated immunoassay platform can be used to analyze at least 240 tests per hour or at least 440 tests per hour.

In some embodiments, methods of detecting biomarkers as proteins in a sample include contacting a sample with one or more antibody agents directed to the biomarkers of interest. In some embodiments, such methods also include contacting the sample with a first set of one or more detection agents. In some embodiments, the antibody agents are labeled with the first set of one or more detection agents. In some embodiments, the first set of one or more detection agents include one or more acridinium ester molecules.

Acridinium ester (AE) molecules can be used to label proteins and nucleic acids. Acridinium-labeled proteins can be used for detection in immunoassays. Exposing AE to an alkaline H₂O₂ (hydrogen peroxide) produces chemiluminescence. Light is emitted at a wavelength maximum in the range of 430 to 480 nm, depending on the specific AE variant. Such light can be detected, for example, by high-efficiency photomultiplier tubes. The light emission is rapid and completes within 1 to 5 seconds. Diversity in AE forms contributes to better assay performance, including improved sensitivity and robustness. AE molecules can be used to label small molecules, large analytes, and antibodies.

Additional methods of detecting biomarkers include methods for detecting biomarkers as nucleic acids. Nucleic acid-based methods of detecting biomarkers include performing nucleic acid amplification methods, such as polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). In some embodiments, a nucleic acid-based method of detecting biomarkers includes detecting hybridization between one or more nucleic acid probes and one or more nucleotides that encode the biomarker of interest. In some embodiments, the nucleic acid probes are each complementary to at least a portion of one of the one or more nucleotides that encode the biomarker of interest. In some embodiments, the nucleotides that encode the biomarker of interest include DNA (e.g., cDNA). In some embodiments, the nucleotides that encode the biomarker of interest include RNA (e.g., mRNA).

In some embodiments, a method provided herein detects a level of one or more ATTR Biomarkers (e.g., a level of two or more, three or more, four or more, or five or more ATTR Biomarkers) to obtain an ATTR Biomarker profile. In some embodiments, an ATTR Biomarker profile includes a level of each of the ATTR Biomarkers being assessed. In some embodiments, an ATTR Biomarker profile includes a level of each of the ATTR Biomarkers detected, e.g., as described herein.

### Sample

In some embodiments, a sample as disclosed herein is a biological sample. In some embodiments, a biological sample is a blood sample, e.g., drawn from an artery or vein of a subject. A blood sample can be a whole blood sample, a plasma sample, or a serum sample. In some embodiments, a biological sample includes cardiac tissue.

In some embodiments, a sample is obtained from a subject. In some embodiments, a subject from which a sample was obtained is being assessed for TTR-CM. In some embodiments, a subject from which a sample was obtained is suffering from or is at risk of developing amyloid transthyretin cardiomyopathy (TTR-CM).

In some embodiments, a method disclosed herein includes obtaining a biological sample from a subject. In some embodiments, obtaining a biological sample from a subject includes drawing blood. In some embodiments, obtaining a biological sample from a subject includes performing a biopsy.

In some embodiments of methods disclosed herein, a sample is provided by, e.g., medical personnel.

### Subject

In some embodiments, a subject as disclosed herein is a mammal. In some embodiments, a mammal is a human.

In some embodiments, a subject as disclosed herein is a biological male. In some embodiments, a subject as disclosed herein is a biological female.

In some embodiments, a subject as disclosed herein is overweight. In some embodiments, a subject has a body mass index (BMI) of 25 or more. In some embodiments, a subject has a body mass index (BMI) of 30 or more.

In some embodiments, a subject is at least 50 years old. In some embodiments, a subject is at least 55 years old. In some embodiments, a subject is at least 60 years old. In some embodiments, a subject is at least 65 years old.

### Methods Utilizing Thresholds

In some methods disclosed herein, a level of one or more ATTR Biomarkers can be compared to a threshold. In some embodiments, methods disclosed herein include a comparison of a level of one or more ATTR Biomarkers to respective thresholds. In some embodiments, methods disclosed herein include a comparison of a level of one or more ATTR Biomarkers to reference thresholds.

A reference threshold may be a threshold from a subject known or independently verified to have good cardiac health, or from a subject known or independently verified to have poor cardiac health, such as is the case for a subject having TTR-CM. Alternately or in combination a subject's ATTR Biomarker profile is compared to a reference threshold determined from a plurality of subject of common known status (e.g., healthy, not diagnosed with TTR-CM, or diagnosed with TTR-CM). In some embodiments, a reference threshold is an average of known level of an ATTR Biomarker from a plurality of subjects, or alternately is a range defined by the range of levels of an ATTR Biomarker observed in reference subjects.

In more complex assessment approaches, a subject's ATTR Biomarker level is compared to a reference ATTR Biomarker level constructed from a larger number of subjects of a common status (e.g., healthy, not diagnosed with TTR-CM, or diagnosed with TTR-CM), such as at least 10, at least 50, at least 100, at least 500, at least 1000 or more subjects. Often, reference subjects are evenly distributed in status between (1) healthy/not diagnosed with TTR-CM and (2) diagnosed with TTR-CM. Assessment includes in some cases iterative or simultaneous comparison of a subject's ATTR Biomarker level to a plurality of profiles of known status.

A plurality of known reference ATTR Biomarker profiles (e.g., detected levels of one or more ATTR Biomarkers in a reference sample) can also be used to train a computational assessment algorithm, e.g., a machine learning model, such that a single comparison between a subject's ATTR Biomarker profile and a reference ATTR Biomarker profile provides an outcome that integrates or aggregates information from a large number of subjects of common known health status (e.g., healthy, not diagnosed with TTR-CM, or diagnosed with TTR-CM), such as at least 10, at least 50, at least 100, at least 500, at least 1000 or more individuals. Generation of such a reference ATTR Biomarker profile can facilitate much faster assessment of a subject's risk of TTR-CM, or assessment using much less computational power.

A reference ATTR Biomarker profile can be generated from a plurality of reference ATTR Biomarker profiles through any of a number of computational approaches known to one of skill in the art. Machine learning models are readily constructed, for example, using any number of statistical programming languages such as R, scripting languages such as Python and associated machine learning packages, data mining software such as Weka or Java, Mathematica, Matlab or SAS.

A subject's ATTR Biomarker profile can be compared to a reference ATTR Biomarker profile as generated above or otherwise by one of skill in the art, and an output assessment is generated. A number of output assessments are consistent with the disclosure herein. Output assessments include a single assessment, often narrowed by a sensitivity, specificity or sensitivity and specificity parameter, indicating a health status assessment (e.g., probability subject has TTR-CM, subject is not at risk of TTR-CM, subject is at risk of TTR-CM, subject has TTR-CM). Alternately or in combination, additional parameters are provided, such as the subject's demographic factors (e.g., one or more of age, weight, biological sex, ethnicity, BMI, medical history, risk factors, family history, and geographic location) and/or the subject's imaging-based biomarkers (e.g., left ventricle septal wall thickness and/or ejection fraction).

More specifically, in some embodiments, methods disclosed herein further include diagnosing a subject with amyloid transthyretin cardiomyopathy (TTR-CM) if the level of at least one of the one or more ATTR Biomarkers (e.g., TnI, PKM1, PKM2, NT-proBNP, RBP4, DCN, TIMP2, SMOC-2, NfL, or combinations thereof) that is detected is above a threshold value. In some embodiments, methods disclosed herein further include diagnosing a subject with amyloid transthyretin cardiomyopathy (TTR-CM) if the level of at least one of the one or more ATTR Biomarkers (e.g., TnI, PKM1, PKM2, NT-proBNP, RBP4, DCN, TIMP2, SMOC-2, NfL, or combinations thereof) that is detected is at least 1.3, at least 1.4, at least 1.5, at least 1.6, at least 1.7, at least 1.8, or at least 1.9 fold greater than a threshold value. In some embodiments, methods disclosed herein further include diagnosing a subject with amyloid transthyretin cardiomyopathy (TTR-CM) if the level of each of the one or more ATTR Biomarkers (e.g., TnI, PKM1, PKM2, NT-proBNP, RBP4, DCN, TIMP2, SMOC-2, NfL, or combinations thereof) that is detected is above a threshold value. In some embodiments, methods disclosed herein further include diagnosing a subject with amyloid transthyretin cardiomyopathy (TTR-CM) if the level of each of the one or more ATTR Biomarkers (e.g., TnI, PKM1, PKM2, NT-proBNP, RBP4, DCN, TIMP2, SMOC-2, NfL, or combinations thereof) that is detected is at least 1.3, at least 1.4, at least 1.5, at least 1.6, at least 1.7, at least 1.8, or at least 1.9 fold greater than a threshold value.

In some embodiments, the methods disclosed herein further include recommending a subject for one or more cardiomyopathy tests if the level of at least one or the one or more ATTR Biomarkers is above threshold value. In some such methods, the subject is recommended for one or more cardiomyopathy tests if the level of at least one of the one or more ATTR Biomarkers that is detected is at least 1.3, at least 1.4, at least 1.5, at least 1.6, at least 1.7, at least 1.8, or at least 1.9 fold greater than a threshold value. In some embodiments, the methods disclosed herein further include recommending a subject for one or more cardiomyopathy tests if the level of each of the one or more ATTR Biomarkers is above threshold value. In some such methods, the subject is recommended for one or more cardiomyopathy tests if the level of each of the one or more ATTR Biomarkers that is detected is at least 1.3, at least 1.4, at least 1.5, at least 1.6, at least 1.7, at least 1.8, or at least 1.9 fold greater than a threshold value.

As discussed above, in some embodiments, methods of detecting one or more ATTR Biomarkers (e.g., ATTR Biomarker proteins) in a sample include contacting a sample with one or more antibody agents directed to the ATTR Biomarker. In some embodiments, such methods also include contacting the sample with a first set of one or more detection agents. In some embodiments, the antibody agents are labeled with the first set of one or more detection agents. In some embodiments, the first set of one or more detection agents include one or more acridinium ester molecules.

Acridinium ester (AE) molecules can be used to label proteins and nucleic acids. Acridinium-labeled proteins can be used for detection in immunoassays. Exposing AE to an alkaline H₂O₂ (hydrogen peroxide) produces chemiluminescence. Light is emitted at a wavelength maximum in the range of 430 to 480 nm, depending on the specific AE variant. Such light can be detected, for example, by high-efficiency photomultiplier tubes.

In some embodiments, detecting binding between an ATTR Biomarker and one or more antibody agents directed against the ATTR Biomarker includes determining absorbance values or emission values for the first set of one or more detection agents. For example, the absorbance values are indicative of the level of binding (e.g., higher absorbance is indicative of more binding). In some embodiments, the absorbance values or emission values for the first set of one or more detection agents are above a threshold value. In some embodiments, the absorbance values or emission values for the first set of one or more detection agents is at least 1.3, at least 1.4, at least 1.5, at least 1.6, at least 1.7, at least 1.8, or at least 1.9 fold greater than a threshold value. In some embodiments, the threshold value is an average of absorbance values or emission values determined for a second set of one or more detection agents that label two or more control samples. In some such embodiments, the second set of one or more detection agents is similar to or the same as the first set of one or more detection agents.

In one aspect, the present disclosure provides a method of detecting one or more ATTR Biomarkers (e.g., TnI, PKM1, PKM2, NT-proBNP, RBP4, DCN, TIMP2, SMOC-2, NfL, or combinations thereof) in a subject, said method including detecting whether one or more ATTR Biomarkers (e.g., TnI, PKM1, PKM2, NT-proBNP, RBP4, DCN, TIMP2, SMOC-2, NfL, or combinations thereof) are present in a sample obtained from the subject according to the methods of detecting biological markers described above. In some embodiments, methods of detecting one or more ATTR Biomarkers include detecting binding between a ATTR Biomarker and one or more anti-ATTR Biomarker antibody agents. In some embodiments, detecting whether one or more ATTR Biomarkers are present in a sample obtained from the subject includes detecting a level of one or more ATTR Biomarkers present in a sample obtained from the subject. In some embodiments, the level of an ATTR Biomarkers that is detected is above a threshold value for the respective ATTR Biomarker(s). In some embodiments, the level of an ATTR Biomarkers that is detected is at least 1.3, at least 1.4, at least 1.5, at least 1.6, at least 1.7, at least 1.8, or at least 1.9 fold greater than a threshold value.

In some embodiments, the present disclosure provides methods of detecting one or more one or more ATTR Biomarkers (e.g., TnI, PKM1, PKM2, NT-proBNP, RBP4, DCN, TIMP2, SMOC-2, NfL, or combinations thereof) in a sample obtained from a subject. Biological markers for amyloid transthyretin cardiomyopathy can include TnI, PKM1, PKM2, NT-proBNP, RBP4, DCN, TIMP2, SMOC-2, NfL, or combinations thereof. In some embodiments, control samples used in the methods described above include samples obtained from one or more subjects who do not have ATTR amyloidosis and/or TTR-CM.

In some embodiments, the methods disclosed herein further include diagnosing the subject with amyloid transthyretin cardiomyopathy (TTR-CM) if the level of at least one or the one or more ATTR Biomarkers (e.g., TnI, PKM1, PKM2, NT-proBNP, RBP4, DCN, TIMP2, SMOC-2, NfL, or combinations thereof) that is detected is above a threshold value. In some embodiments, the method includes diagnosing a subject with TTR-CM if the level of at least one or the one or more ATTR Biomarkers that is detected is at least 1.3, at least 1.4, at least 1.5, at least 1.6, at least 1.7, at least 1.8, or at least 1.9 fold greater than a threshold value.

In some embodiments, the methods disclosed herein further include diagnosing the subject with amyloid transthyretin cardiomyopathy (TTR-CM) if the absorbance values or emission values for the first set of one or more detection agents is above a threshold value. In some embodiments, the method includes diagnosing a subject with TTR-CM if the absorbance values or emission values for the first set of one or more detection agents is at least 1.3, at least 1.4, at least 1.5, at least 1.6, at least 1.7, at least 1.8, or at least 1.9 fold greater than a threshold value. In some embodiments, the threshold value is an average of absorbance values or emission values determined for a second set of one or more detection agents that label two or more control samples. In some such embodiments, the second set of one or more detection agents is similar to or the same as the first set of one or more detection agents.

In some embodiments, the methods disclosed herein further include recommending a subject for one or more cardiomyopathy tests if the level of at least one or the one or more ATTR Biomarkers is above threshold value. In some such methods, the subject is recommended for one or more cardiomyopathy tests if the level of at least one or the one or more ATTR Biomarkers that is detected is at least 1.3, at least 1.4, at least 1.5, at least 1.6, at least 1.7, at least 1.8, or at least 1.9 fold greater than a threshold value.

In some embodiments, the methods disclosed herein further include recommending the subject for one or more cardiomyopathy tests if the absorbance values or emission values for the first set of one or more detection agents is above a threshold value. In some such methods, the subject is recommended for one or more cardiomyopathy tests if the absorbance values or emission values for the first set of one or more detection agents is at least 1.3, at least 1.4, at least 1.5, at least 1.6, at least 1.7, at least 1.8, or at least 1.9 fold greater than a threshold value.

Cardiomyopathy tests that may be used according to the methods of the present disclosure include echocardiogram or advanced imaging methods. In some embodiments, the advanced imaging methods include cardiac magnetic resonance imaging (CMR) or scintigraphy (e.g. using a radioisotope conjugate such as 99m Tc-Pyrophosphate and/or using single photon emission computed tomography (SPECT). In some embodiments the methods disclosed herein further include recommending the subject for cardiac biopsy if the level of at least one or the one or more ATTR Biomarkers that is detected is above a threshold value and the subject tests positive for cardiomyopathy in one or more of the cardiomyopathy tests. In some such embodiments, the biopsy tissue is tested for cardiomyopathy by immunochemical staining. For example, immunochemical staining includes use of one or more antibody agents to kappa or lambda light chain amyloid deposits in heart tissue and/or one or more antibody agents to transthyretin deposits. A subject can be diagnosed with amyloid transthyretin cardiomyopathy (TTR-CM) if immunochemical staining indicates presence of transthyretin deposits in the cardiac tissue.

In any of the above embodiments, a threshold value may be an average of values detected for two or more control samples. In some such embodiments, the values detected for the two or more control samples represent control levels for one or more ATTR Biomarkers. In some embodiments, the control samples include recombinant ATTR Biomarkers (e.g., TnI, PKM1, PKM2, NT-proBNP, RBP4, DCN, TIMP2, SMOC-2, NfL, or combinations thereof). In some embodiments, the two or more control samples are each a sample obtained from a subject who does not have TTR-CM. In some embodiments, the threshold value is a value reported in a standard table.

### Methods Utilizing ATTR Biomarker Profiles

An algorithm-based assay and associated information provided by the practice of any of the methods described herein can facilitate optimal treatment and decision making for subjects. For example, methods described herein can enable a physician or caretaker to identify patients who have a low likelihood of having TTR-CM and therefore would not need treatment, would not need additional cardiac tests, or would not need increased monitoring for TTR-CM, or who have a high likelihood of having TTR-CM, would need treatment, would need additional cardiac tests, or would need increased monitoring for TTR-CM.

An ATTR Biomarker score can be determined by the application of a specific algorithm in some cases. In some embodiments, an ATTR Biomarker score is quantitative. The algorithm used to calculate the ATTR Biomarker score in the methods disclosed herein may group the expression level values of an ATTR Biomarker or groups of ATTR Biomarkers. The formation of a particular group of ATTR Biomarkers, in addition, can facilitate the mathematical weighting of the contribution of various expression levels of ATTR Biomarker or ATTR Biomarker subsets (for example classifier) to the quantitative score.

Exemplary ATTR Biomarkers and corresponding amino acid sequences are listed in Table 1. Exemplary combinations of ATTR Biomarkers are listed in Tables 2-5.

Methods described herein, as well as kits and systems provided herein, can utilize an algorithm-based diagnostic assay for predicting if a subject from which the sample was obtained is at risk of or suffering from TTR-CM, selecting a subject from which the sample was obtained for one or more cardiomyopathy tests, and/or selecting a subject from which the sample was obtained to receive one or more doses of a TTR stabilizing agent.

Levels of one or more ATTR Biomarkers, and optionally one or more demographic factors (e.g., one or more of age, weight, biological sex, ethnicity, BMI, medical history, risk factors, family history, and geographic location) and/or imaging-based biomarkers (e.g., left ventricle septal wall thickness and/or ejection fraction) can be used alone or arranged into functional subsets to calculate an ATTR Biomarker score that is used to predict if a subject from which the sample was obtained is at risk of or suffering from TTR-CM, select a subject from which the sample was obtained for one or more cardiomyopathy tests, and/or select a subject from which the sample was obtained to receive one or more doses of a TTR stabilizing agent.

Methods disclosed herein include using an ATTR Biomarker profile to compute an ATTR Biomarker score. In some embodiments, using an ATTR Biomarker profile to compute an ATTR Biomarker score includes applying an algorithm to the ATTR Biomarker profile to compute an ATTR Biomarker score. In some embodiments, an algorithm is or is derived from a decision tree methodology, a neural boosted methodology, a bootstrap forest methodology, a boosted tree methodology, a K nearest neighbors methodology, a generalized regression forward selection methodology, a generalized regression pruned forward selection methodology, a fit stepwise methodology, a generalized regression lasso methodology, a generalized regression elastic net methodology, a generalized regression ridge methodology, a nominal logistic methodology, a support vector machines methodology, a discriminant methodology, a naïve Bayes methodology, or a combination thereof. In some embodiments, an algorithm is or is derived from a decision tree methodology, a neural boosted methodology, a bootstrap forest methodology, a boosted tree methodology, a generalized regression lasso methodology, a generalized regression elastic net methodology, a generalized regression ridge methodology, a nominal logistic methodology, a support vector machines methodology, a discriminant methodology, or a combination thereof. In some embodiments, an algorithm is or is derived from a decision tree methodology, a neural boosted methodology, a bootstrap forest methodology, a boosted tree methodology, a support vector machines methodology, or a combination thereof.

Additional algorithms can be used in methods provided herein, and the algorithms provided above are merely exemplary of the types of algorithms that can be used to generate an ATTR Biomarker score. Exemplary algorithms have been described, e.g., by Duda, 2001, Pattern Classification, John Wiley & Sons, Inc., New York. pp. 396-408 and pp. 411-412; and Hastie et al., 2001, The Elements of Statistical Learning, Springer-Verlag, New York, Chapter 9, each of which is hereby incorporated by reference. Moreover, as indicated above, combinations of algorithms can be used in methods provided herein. For example, a boosted tree methodology can be a combination of a decision tree methodology and a boosting methodology. Further combinations are possible and are contemplated for use in methods provided herein. Exemplary algorithms that can be used in the methods provided herein are described below.

### Decision Tree

One methodology that can be used to calculate an ATTR Biomarker score from an ATTR Biomarker profile is a decision tree. A decision tree can be constructed using a training population and specific data analysis algorithms. Decision trees are described generally by Duda, 2001, Pattern Classification, John Wiley & Sons, Inc., New York. pp. 395-396, which is hereby incorporated by reference. Tree-based methods partition the feature space into a set of rectangles, and then fit a model (like a constant) in each one.

A training population data can include ATTR Biomarker profiles (e.g., including a level of one or more ATTR Biomarkers in a sample) across a training set population. One specific algorithm that can be used to construct a decision tree is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York. pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests-Random Features,” Technical Report 567, Statistics Department, U.C. Berkeley, September 1999, which is hereby incorporated by reference in its entirety.

An aim of a decision tree is to induce a classifier (a tree) from real-world example data. This tree can be used to classify unseen examples that have not been used to derive the decision tree. As such, a decision tree can be derived from training data. Exemplary training data contains data for a plurality of subjects (e.g., a training population). An ATTR Biomarker profile can be provided and/or used for each respective subject. In some embodiments, training data includes ATTR Biomarker proles the training population.

The following algorithm describes an exemplary decision tree derivation:

### Tree (Examples,Class,Features)

Create a root node
If all Examples have the same Class value, give the root this label
Else if Features is empty label the root according to the most common value
Else begin
Calculate the information gain for each Feature
Select the Feature A with highest information gain and make this the root Feature
For each possible value, v, of this Feature
   Add a new branch below the root, corresponding to A=v
   Let Examples(v) be those examples with A=v
   If Examples(v) is empty, make the new branch a leaf node labeled with the most common value among Examples
   Else let the new branch be the tree created by
   Tree(Examples(v),Class,Features—{A})
end

In univariate decision trees, each split is based on a feature value (e.g., a level) for a corresponding biomarker. Further, multivariate decision trees can be implemented in a method described herein. Multivariate decision trees are described in Duda, 2001, Pattern Classification, John Wiley & Sons, Inc., New York, pp. 408-409, which is hereby incorporated by reference. In such multivariate decision trees, some or all of the decisions include a linear combination of feature values (e.g., levels) for a plurality of ATTR Biomarkers of an ATTR profile. Such a linear combination can be trained using known techniques such as gradient descent on a classification or by the use of a sum-squared-error criterion.

As an illustrative example, the following expression is used:

$$0.05 ( X 1 ) + 0.2 ( X 2 ) < 500$$

In this example, X1 and X2 refer to two different features (e.g., levels) for two different ATTR Biomarkers. To apply the methodology, the values of features X1 and X2 (e.g., as part of an ATTR Biomarker profile) are obtained from the measurements obtained from an unclassified subject. These values are then inserted into the equation. If a value of less than 500 is computed, then a first branch in the decision tree is taken. Otherwise, a second branch in the decision tree is taken.

Bagging, boosting, and additive trees can be combined with a decision methodology to improve weak decision rules. These techniques are designed for, and usually applied to, decision trees, such as the decision trees described above. In addition, such techniques can also be useful in decision rules developed using other types of data analysis algorithms such as linear discriminant analysis.

In bagging, a training set is sampled, generating random independent bootstrap replicates, constructing a decision rule on each of these, and aggregateing them by a simple majority vote in the final decision rule. See, for example, Breiman, 1996, Machine Learning 24, 123-140; and Efron & Tibshirani, An Introduction to Boostrap, Chapman & Hall, New York, 1993, which is hereby incorporated by reference in its entirety.

In boosting, decision rules are constructed on weighted versions of the training set, which are dependent on previous classification results. Initially, all features under consideration have equal weights, and the first decision rule is constructed on this data set. Then, weights are changed according to the performance of the decision rule. Erroneously classified features get larger weights, and the next decision rule is boosted on the reweighted training set. In this way, a sequence of training sets and decision rules is obtained, which is then combined by simple majority voting or by weighted majority voting in the final decision rule. See, for example, Freund & Schapire, “Experiments with a new boosting algorithm,” Proceedings 13th International Conference on Machine Learning, 1996, 148-156, which is hereby incorporated by reference in its entirety.

Measurement data used in the methods, systems, kits and compositions disclosed herein are optionally normalized. Normalization refers to a process to correct for example, differences in the amount of genes or protein levels assayed and variability in the quality of the template used, to remove unwanted sources of systematic variation measurements involved in the processing and detection of genes or protein expression. Other sources of systematic variation are attributable to laboratory processing conditions.

In some instances, normalization methods are used for the normalization of laboratory processing conditions. Non-limiting examples of normalization of laboratory processing that may be used with methods of the disclosure include but are not limited to: accounting for systematic differences between the instruments, reagents, and equipment used during the data generation process, and/or the date and time or lapse of time in the data collection.

Assays can provide for normalization by incorporating the expression of certain normalizing standard genes or proteins, which do not significantly differ in expression levels under the relevant conditions, that is to say they are known to have a stabilized and consistent expression level in that particular sample type. Suitable normalization genes and proteins that can be used with the present disclosure include housekeeping genes. (See, for example, E. Eisenberg, et al., Trends in Genetics 19(7):362-365 (2003). In some applications, the normalizing biomarkers (genes and proteins), also referred to as reference genes, known not to exhibit meaningfully different expression levels in subjects with TTR-CM as compared to control subjects without TTR-CM. In some applications, it may be useful to add a stable isotope labeled standards which can be used and represent an entity with known properties for use in data normalization.

In other applications, a standard, fixed sample can be measured with each analytical batch to account for instrument and day-to-day measurement variability.

Machine learning algorithms for sub-selecting discriminating biomarkers and optionally subject characteristics, and for building classification models, are used in some methods and systems herein to determine clinical outcome scores. Examples of such algorithms are described above. These algorithms can aid in selection of important biomarker features and transform the underlying measurements into a score or probability relating to, for example, clinical outcome, disease risk, disease likelihood, presence or absence of disease, treatment response, and/or classification of disease status.

An ATTR Biomarker Score can be determined by comparing a subject-specific ATTR Biomarker profile to a reference ATTR Biomarker profile. A reference ATTR Biomarker profile can be representative a known diagnosis. For example, a ATTR Biomarker profile can represent a positive diagnosis of TTR-CM. As another example, a reference ATTR Biomarker profile can represent a negative diagnosis of TTR-CM. In some cases, an increase in a score indicates an increased likelihood of one or more of: a poor clinical outcome, good clinical outcome, high risk of disease, low risk of disease, complete response, partial response, stable disease, non-response, and recommended treatments for disease management. In some cases, a decrease in the quantitative score indicates an increased likelihood of one or more of: a poor clinical outcome, good clinical outcome, high risk of disease, low risk of disease, complete response, partial response, stable disease, non-response, and recommended treatments for disease management.

A similar ATTR Biomarker profile from a subject to a reference ATTR Biomarker profile often indicates an increased likelihood of one or more of: a poor clinical outcome, good clinical outcome, high risk of disease, low risk of disease, complete response, partial response, stable disease, non-response, and recommended treatments for disease management. In some applications, a dissimilar ATTR Biomarker profile between a subject and a reference indicates one or more of: an increased likelihood of a poor clinical outcome, good clinical outcome, high risk of disease, low risk of disease, complete response, partial response, stable disease, non-response, and recommended treatments for disease management.

Results can be provided to a subject, a health care professional or other professional. Results are optionally accompanied by a heath recommendation, such as a recommendation to confirm or independently assess TTR-CM risk, for example using one or more cardiomyopathy tests.

A recommendation optionally includes information relevant to a treatment regimen, such as information indicating that a treatment regimen. Efficacy of a regimen can be assessed in some cases by comparison of a subject's ATTR Biomarker profile at a first time point, optionally prior to a treatment and a later second time point, optionally subsequent to a treatment instance. ATTR Biomarker profiles can be compared to one another, each to a reference, or otherwise assessed so as to determine whether a treatment regimen demonstrates efficacy such that it should be continued, increased, replaced with an alternate regimen or discontinued because of its success in addressing TTR-CM or associated signs and symptoms. Some assessments rely upon comparison of a subject's ATTR Biomarker profile at multiple time points, such as at least one time point prior to a treatment and at least one time point following a treatment. ATTR Biomarker profiles can be compared one to another or to at least one reference biomarker panel level or both to one another and to at least one reference biomarker panel level.

### Treatment for Amyloid Transthyretin Cardiomyopathy

Therapeutic approaches for ATTR amyloidosis include reducing production of TTR, inhibiting or reducing aggregation of TTR, inhibiting or reducing TTR fibril or amyloid formation, reducing or clearing TTR deposits, and stabilizing non-toxic conformations of TTR (e.g., tetrameric forms). In some embodiments, the methods disclosed herein further include detecting a level of each of one or more ATTR Biomarkers (e.g., TnI, PKM1, PKM2, NT-proBNP, RBP4, DCN, TIMP2, SMOC-2, NfL, or combinations thereof) in a sample obtained from a subject and administering an effective amount of a transthyretin (TTR) stabilizing agent to the diagnosed subject. In some embodiments, the TTR stabilizing agent is Tafamidis.

In some embodiments, the present disclosure includes a method for selecting a patient for treatment with a TTR stabilizing agent including the step of detecting a level of each of one or more ATTR Biomarkers (e.g., TnI, PKM1, PKM2, NT-proBNP, RBP4, DCN, TIMP2, SMOC-2, NfL, or combinations thereof) in a sample obtained from the subject.

In some embodiments, the present disclosure includes a method of treating TTR-CM in subject at risk of or suffering from TTR-CM, the method including administering to the subject a therapeutically effective amount of a TTR stabilizing agent, wherein the subject expresses a level of an ATTR Biomarker or ATTR Biomarker gene product above a threshold value. In some embodiments, a method of treating TTR-CM in subject at risk of or suffering from TTR-CM includes administering to the subject a therapeutically effective amount of a TTR stabilizing agent, wherein the subject expresses a level of more than one ATTR Biomarker or ATTR Biomarker gene product above a threshold value. In some embodiments, the method further includes determining that the subject expresses a level of an ATTR Biomarker or ATTR Biomarker gene product above a threshold value. In some embodiments, prior to administration, the subject has been determined to express a level of an ATTR Biomarker or ATTR Biomarker gene product above a threshold value.

In some embodiments, the biomarkers disclosed herein can be used for screening patients for effective therapies for ATTR amyloidosis and TTR-CM. In some embodiments, a therapy for TTR-CM is a stabilizer of the TTR tetramer. Examples of TTR stabilizers include tafamidis and diflunisal.

Another approach to therapy is reducing total TTR production. For example, antisense oligonucleotide (ASO)-based therapy and RNA interference (RNAi) are therapeutic approaches to lowering total TTR. ISIS-TTR_{Rx} is an ASO-based therapy that causes destruction of both wild-type and mutant forms of the TTR transcript. Other possible therapeutic agents include ALN-TTR02 (Patisiran), ALN-TTRsc (Revurisan), doxycycline, tauroursodeoxycholic acid (TUDCA), the combination of doxycycline and TUDCA, epigallocatechin gallate (EGCG), curcumin, or resveratrol. Antibodies targeting TTR can also be used as a therapy; for example by antibody-mediated inhibition of TTR aggregation and fibril formation; antibody-mediated stabilization of non-toxic conformations of TTR (e.g., tetrameric forms); or antibody-mediated clearance of aggregated TTR, oligomeric TTR, or monomeric TTR. Additionally, antibodies to TTR can be coupled or conjugated to therapeutic agents, for example, to target TTR.

### Kits

Also provided by the present disclosure are kits including one or more anti-ATTR Biomarker agents and instructions for use (e.g., treatment, prophylactic, or diagnostic use). In some embodiments, the kit is used for an in vitro diagnostic assay to diagnose TTR-CM (including TTR-CM resulting from wild-type ATTR amyloidosis). In some embodiments, the kits of the disclosure further include a TTR stabilizing agent (e.g., Tafamidis).

In some embodiments, the one or more anti-ATTR Biomarker agents include antibody agents. In some embodiments, one or more of the antibody agents are labeled with a detectable moiety. In some embodiments, the kit further includes a detection agent (e.g., one or more acridinium ester molecules). In some embodiments, one or more of the antibody agents are labeled with one or more of the acridinium ester molecules. In some embodiments, the kit further includes one or more secondary antibody agents that specifically bind to one or more of the anti-ATTR Biomarker antibody agents.

In some embodiments, the one or more anti-ATTR Biomarker agents include nucleic acid probes. In some embodiments, at least a portion of each nucleic acid probe hybridizes to one or more portions of a nucleotide that encodes an ATTR Biomarker (e.g., TnI, PKM1, PKM2, NT-proBNP, RBP4, DCN, TIMP2, SMOC-2, NfL, or combinations thereof). Nucleotides that encode an ATTR Biomarker can be DNA (e.g., cDNA) or RNA (e.g. mRNA). In some embodiments, the nucleic acid probes are labeled with one or more detection agents (e.g., wherein the detection agents indicate presence of nucleotides that encode an ATTR Biomarker).

In some embodiments, the kit further includes one or more control samples. In some embodiments, the control samples include one or more ATTR Biomarker standards. In some embodiments, an ATTR Biomarker standard includes recombinant an ATTR Biomarker (e.g., TnI, PKM1, PKM2, NT-proBNP, RBP4, DCN, TIMP2, SMOC-2, NfL, or combinations thereof). In some embodiments, an ATTR Biomarker standard includes synthetic ATTR Biomarker (e.g., TnI, PKM1, PKM2, NT-proBNP, RBP4, DCN, TIMP2, SMOC-2, NfL, or combinations thereof) nucleic acids.

In addition to the above, a kit can include other ingredients, such as a solvent or buffer, a stabilizer or a preservative, and/or an agent for treating a condition or disorder described herein. Alternatively, other ingredients can be included in a kit, but in different compositions or containers than the anti-ATTR Biomarker agents. In such embodiments, a kit can include instructions for admixing the anti-ATTR Biomarker agents and the other ingredients, or for using the anti-ATTR Biomarker together with the other ingredients.

In certain embodiments, kits for use in accordance with the present disclosure may include, a reference or control sample(s), instructions for processing samples, performing tests on samples, instructions for interpreting the results, buffers and/or other reagents necessary for performing tests.

The present disclosure also provides that recognition that certain single ATTR Biomarkers can be helpful for detecting and/or diagnosing ATTR amyloidosis or TTR-CM. The present disclosure further provides the insight that particular combinations of ATTR Biomarkers are especially useful for detecting and/or diagnosing ATTR amyloidosis or TTR-CM. Thus, methods, compositions, and kits described herein can be used for assays to assess the risk of TTR-CM, assess whether a subject should undergo further cardiac tests, and/or diagnose TTR-CM based on detection or measurement of ATTR Biomarkers in a sample, e.g., a biological sample obtained from a subject.

Methods and kits provided herein are able to detect TTR-CM in a sample with a sensitivity and a specificity that renders the outcome of the test reliable enough to be medically actionable. Methods and kits described herein for detection and/or diagnosis of TTR-CM in a subject detects TTR-CM with a sensitivity greater than 75%, greater than 80%, greater than 85%, greater than 90%, greater than 95%, greater than 96%, greater than 97%, greater than 98%, greater than 99%, or about 100%. In some embodiments, methods and kits provided herein can detect TTR-CM with a sensitivity that is between about 70%-100%, between about 80%-100%, or between about 90-100%. In some embodiments, methods and kits provided herein can detect TTR-CM with a specificity greater than 70%, greater than 75%, greater than 80%, greater than 85%, greater than 90%, greater than 95%, greater than 96%, greater than 97%, greater than 98%, greater than 99%, or about 100%. In some embodiments, methods and kits provided herein can detect TTR-CM with a specificity that is between about 50%-100%, between about 60%-100%, between about 70%-100%, between about 80%-100%, or between about 90-100%. In some embodiments, methods and kits provided herein can detect TTR-CM with a sensitivity and a specificity that is 50% or greater, 60% or greater, 70% or greater, 75% or greater, 80% or greater, 85% or greater, 90% or greater. In some embodiments, methods and kits provided herein can detect TTR-CM with a sensitivity and a specificity that is between about 50%-100%, between about 60%-100%, between about 70%-100%, between about 80%-100%, or between about 90-100%.

### Compositions

Also provided herein are compositions. In some embodiments, a composition includes one or more ATTR Biomarkers and one or more anti-ATTR Biomarker agents. In some embodiments, one or more ATTR Biomarkers include TnI, PKM1, PKM2, NT-proBNP, RBP4, DCN, TIMP2, SMOC-2, NfL, or a combinations thereof, and one or more anti-ATTR Biomarker agents include an anti-TnI agent, an anti-PKM1 agent, an anti-PKM2 agent, an anti-NT-proBNP agent, an anti-RBP4 agent, an anti-TIMP2 agent, an anti-NfL agent, or a combination thereof.

In some embodiments, a composition includes a combination (e.g., one or more, two or more, three or more, four or more, five or more, etc.) of ATTR Biomarkers and a corresponding combination of anti-ATTR Biomarker agents.

In some embodiments, a composition includes two or more ATTR Biomarkers and two or more anti-ATTR Biomarker agents. In some embodiments, two or more ATTR Biomarkers include TnI, PKM1, PKM2, NT-proBNP, RBP4, DCN, TIMP2, SMOC-2, NfL, or a combinations thereof, and two or more anti-ATTR Biomarker agents include an anti-TnI agent, an anti-PKM1 agent, an anti-PKM2 agent, an anti-NT-proBNP agent, an anti-RBP4 agent, an anti-TIMP2 agent, an anti-NfL agent, or a combination thereof.

In some embodiments, a composition includes three or more ATTR Biomarkers and three or more anti-ATTR Biomarker agents. In some embodiments, three or more ATTR Biomarkers include TnI, PKM1, PKM2, NT-proBNP, RBP4, DCN, TIMP2, SMOC-2, NfL, or a combinations thereof. In some embodiments, three or more anti-ATTR Biomarker agents include an anti-TnI agent, an anti-PKM1 agent, an anti-PKM2 agent, an anti-NT-proBNP agent, an anti-RBP4 agent, an anti-TIMP2 agent, an anti-NfL agent, or a combination thereof.

In some embodiments, a composition includes four or more ATTR Biomarkers and four or more anti-ATTR Biomarker agents. In some embodiments, four or more ATTR Biomarkers include TnI, PKM1, PKM2, NT-proBNP, RBP4, DCN, TIMP2, SMOC-2, NfL, or a combinations thereof. In some embodiments, four or more anti-ATTR Biomarker agents include an anti-TnI agent, an anti-PKM1 agent, an anti-PKM2 agent, an anti-NT-proBNP agent, an anti-RBP4 agent, an anti-TIMP2 agent, an anti-NfL agent, or a combination thereof.

In some embodiments, a composition includes five or more ATTR Biomarkers and five or more anti-ATTR Biomarker agents. In some embodiments, five or more ATTR Biomarkers include TnI, PKM1, PKM2, NT-proBNP, RBP4, DCN, TIMP2, SMOC-2, NfL, or a combinations thereof. In some embodiments, five or more anti-ATTR Biomarker agents include an anti-TnI agent, an anti-PKM1 agent, an anti-PKM2 agent, an anti-NT-proBNP agent, an anti-RBP4 agent, an anti-TIMP2 agent, an anti-NfL agent, or a combination thereof.

In some embodiments, a composition includes a combination (e.g., one or more, two or more, three or more, four or more, etc.) of ATTR Biomarkers and a corresponding combination of anti-ATTR Biomarker agents. In some embodiments, a composition includes a combination of ATTR Biomarkers from Tables 2-5 and a corresponding combination of anti-ATTR Biomarker agents. In some embodiments, a composition includes a combination (e.g., one or more, two or more, three or more, four or more, etc.) of ATTR Biomarkers from Table 2 and a corresponding combination of anti-ATTR Biomarker agents. In some embodiments, a combination (e.g., one or more, two or more, three or more, four or more, etc.) of ATTR Biomarkers from Table 3 and a corresponding combination of anti-ATTR Biomarker agents. In some embodiments, a combination (e.g., one or more, two or more, three or more, four or more, etc.) of ATTR Biomarkers from Table 4 and a corresponding combination of anti-ATTR Biomarker agents. In some embodiments, a combination (e.g., one or more, two or more, three or more, four or more, etc.) of ATTR Biomarkers from Table 5 and a corresponding combination of anti-ATTR Biomarker agents.

In some embodiments, a composition includes TnI and an anti-TnI agent. In some embodiments, a composition includes TnI, PKM1, an anti-TnI agent, and an anti-PKM1 agent. In some embodiments, a composition includes TnI, PKM2, an anti-TnI agent, and an anti-PKM2 agent. In some embodiments, a composition includes TnI, PKM1, PKM2, an anti-TnI agent, an anti-PKM1 agent, and an anti-PKM2 agent. In some embodiments, a composition includes NT-proBNP, RBP4, or both, and an anti-NT-proBNP agent, an anti-RBP4 agent, or both. In some embodiments, a composition includes TIMP2, NfL, or both, and an anti-TIMP2 agent, an anti-NfL agent, or both.

In some embodiments, a composition includes NT-proBNP and an anti-NT-proBNP agent. In some embodiments, a composition includes TnI, PKM1, PKM2, RBP4, or a combination thereof, and an anti-TnI agent, an anti-PKM1 agent, an anti-PKM2 agent, an anti-RBP4 agent, or a combination thereof. In some embodiments, a composition includes TIMP2, NfL, or both, and an anti-TIMP2 agent, an anti-NfL agent, or both.

In some embodiments, a combination (e.g., one or more, two or more, three or more, four or more, etc.) of ATTR Biomarkers includes TnI, PKM2, NT-proBNP, RBP4, an anti-TnI agent, an anti-PKM2 agent, an anti-NT-proBNP agent, and an anti-RBP4 agent. In some embodiments, a combination (e.g., one or more, two or more, three or more, four or more, etc.) of ATTR Biomarkers includes TnI, PKM2, NT-proBNP, an anti-TnI agent, an anti-NT-proBNP agent, and an anti-RBP4 agent. In some embodiments, a combination (e.g., one or more, two or more, three or more, four or more, etc.) of ATTR Biomarkers includes TnI, PKM2, RBP4, an anti-TnI agent, an anti-PKM2 agent, and an anti-RBP4 agent. In some embodiments, a combination (e.g., one or more, two or more, three or more, four or more, etc.) of ATTR Biomarkers includes TnI, PKM1, NT-proBNP, an anti-TnI agent, an anti-PKM1 agent, and an anti-NT-proBNP agent. In some embodiments, a combination (e.g., one or more, two or more, three or more, four or more, etc.) of ATTR Biomarkers includes TnI, PKM1, RBP4, an anti-TnI agent, an anti-PKM1 agent, and an anti-RBP4 agent. In some embodiments, a combination (e.g., one or more, two or more, three or more, four or more, etc.) of ATTR Biomarkers includes TnI, PKM1, PKM2, NT-proBNP, RBP4, an anti-TnI agent, an anti-PKM1 agent, an anti-PKM2 agent, an anti-NT-proBNP agent, and an anti-RBP4 agent. In some embodiments, a combination (e.g., one or more, two or more, three or more, four or more, etc.) of ATTR Biomarkers includes TnI, PKM1, PKM2, NT-proBNP, an anti-TnI agent, an anti-PKM1 agent, an anti-PKM2 agent, and an anti-NT-proBNP agent. In some embodiments, a combination (e.g., one or more, two or more, three or more, four or more, etc.) of ATTR Biomarkers includes TnI, PKM1, PKM2, RBP4, an anti-TnI agent, an anti-PKM1 agent, an anti-PKM2 agent, and an anti-RBP4 agent. In some embodiments, a combination (e.g., one or more, two or more, three or more, four or more, etc.) of ATTR Biomarkers includes NT-proBNP, RBP4, TnI, an anti-TnI agent, an anti-NT-proBNP agent, and an anti-RBP4 agent. In some embodiments, a combination (e.g., one or more, two or more, three or more, four or more, etc.) of ATTR Biomarkers includes RBP4, SMOC-2, TnI, an anti-TnI agent, an anti-RBP4 agent, and an anti-SMOC-2 agent. In some embodiments, a combination (e.g., one or more, two or more, three or more, four or more, etc.) of ATTR Biomarkers includes DCN, NT-proBNP, TnI, an anti-TnI agent, an anti-NT-proBNP agent, and an anti-DCN agent. In some embodiments, a combination (e.g., one or more, two or more, three or more, four or more, etc.) of ATTR Biomarkers includes DCN, RBP4, TnI, an anti-TnI agent, an anti-RBP4 agent, and an anti-DCN agent. In some embodiments, a combination (e.g., one or more, two or more, three or more, four or more, etc.) of ATTR Biomarkers includes NT-proBNP, SMOC-2, TnI, an anti-TnI agent, an anti-NT-proBNP agent, and an anti-SMOC-2 agent. In some embodiments, a combination (e.g., one or more, two or more, three or more, four or more, etc.) of ATTR Biomarkers includes NT-proBNP, TIMP2, TnI, an anti-TnI agent, an anti-NT-proBNP agent, and an anti-TIMP2 agent. In some embodiments, a combination (e.g., one or more, two or more, three or more, four or more, etc.) of ATTR Biomarkers includes NT-proBNP, TnI, an anti-TnI agent, and an anti-NT-proBNP agent. In some embodiments, a combination (e.g., one or more, two or more, three or more, four or more, etc.) of ATTR Biomarkers includes DCN, TnI, an anti-TnI agent, and an anti-DCN agent. In some embodiments, a combination (e.g., one or more, two or more, three or more, four or more, etc.) of ATTR Biomarkers includes DCN, TIMP2, TnI, an anti-TnI agent, an anti-TIMP2 agent, and an anti-DCN agent. In some embodiments, a combination (e.g., one or more, two or more, three or more, four or more, etc.) of ATTR Biomarkers includes RBP4, TnI, an anti-TnI agent, and an anti-RBP4 agent. In some embodiments, a combination (e.g., one or more, two or more, three or more, four or more, etc.) of ATTR Biomarkers includes RBP4, TIMP2, TnI, an anti-TnI agent, an anti-RBP4 agent, and an anti-TIMP2 agent. In some embodiments, a combination (e.g., one or more, two or more, three or more, four or more, etc.) of ATTR Biomarkers includes DCN, SMOC-2, TnI, an anti-TnI agent, an anti-SMOC-2 agent, and an anti-DCN agent. In some embodiments, a combination (e.g., one or more, two or more, three or more, four or more, etc.) of ATTR Biomarkers includes TIMP2, TnI, an anti-TnI agent, and an anti-TIMP2 agent. In some embodiments, a combination (e.g., one or more, two or more, three or more, four or more, etc.) of ATTR Biomarkers includes SMOC-2, TIMP2, TnI, an anti-TnI agent, an anti-SMOC-2 agent, and an anti-TIMP2 agent.

In some embodiments, one or more anti-ATTR Biomarker agents in a composition provided herein include antibody agents. In some embodiments, one or more of the antibody agents are labeled with a detectable moiety. In some embodiments, the kit further includes a detection agent (e.g., one or more acridinium ester molecules). In some embodiments, one or more of the antibody agents are labeled with one or more of the acridinium ester molecules. In some embodiments, the kit further includes one or more secondary antibody agents that specifically bind to one or more of the anti-ATTR Biomarker antibody agents.

In some embodiments, one or more anti-ATTR Biomarker agents in a composition provided herein include nucleic acid probes. In some embodiments, at least a portion of each nucleic acid probe hybridizes to one or more portions of a nucleotide that encodes an ATTR Biomarker (e.g., TnI, PKM1, PKM2, NT-proBNP, RBP4, DCN, TIMP2, SMOC-2, NfL, or combinations thereof). Nucleotides that encode an ATTR Biomarker can be DNA (e.g., cDNA) or RNA (e.g. mRNA). In some embodiments, the nucleic acid probes are labeled with one or more detection agents (e.g., wherein the detection agents indicate presence of nucleotides that encode an ATTR Biomarker).

In some embodiments, a composition includes one or more control samples. In some embodiments, the control samples include one or more ATTR Biomarker standards. In some embodiments, an ATTR Biomarker standard includes recombinant an ATTR Biomarker (e.g., TnI, PKM1, PKM2, NT-proBNP, RBP4, DCN, TIMP2, SMOC-2, NfL, or combinations thereof). In some embodiments, an ATTR Biomarker standard includes synthetic ATTR Biomarker (e.g., TnI, PKM1, PKM2, NT-proBNP, RBP4, DCN, TIMP2, SMOC-2, NfL, or combinations thereof) nucleic acids.

In addition to the above, a composition can include other ingredients, such as a solvent or buffer, a stabilizer or a preservative, and/or an agent for treating a condition or disorder described herein.

### Computer Systems

Methods described herein can be implemented in a computer system having a processor that executes specific instructions in a computer program. In some embodiments, a computer system may be arranged to output an ATTR Biomarker score based on receiving an ATTR Biomarker profile and/or a level of two or more ATTR Biomarkers. Particularly, a computer program may include instructions for the system to select appropriate next steps, including additional medication (e.g., a TTR stabilizer), a treatment, and/or additional testing (e.g., cardiomyopathy tests) for a subject.

In some embodiments, the computer program may be configured such that the computer system can identify a subject for further testing (e.g., cardiomyopathy tests), identify a subject as being at risk or having TTR-CM, and/or identify a subject to receive medication (e.g., a TTR stabilizer) based on received data (e.g., an ATTR Biomarker profile) and use the data to calculate an ATTR Biomarker score. A system may be able to rank-order identified next steps based on an ATTR Biomarker profile with demographic factors and/or imaging-based biomarkers. A system may be able to adjust the rank ordering based on, e.g., a clinical response of a subject or of a family member of the subject who has or is suspected of having TTR-CM.

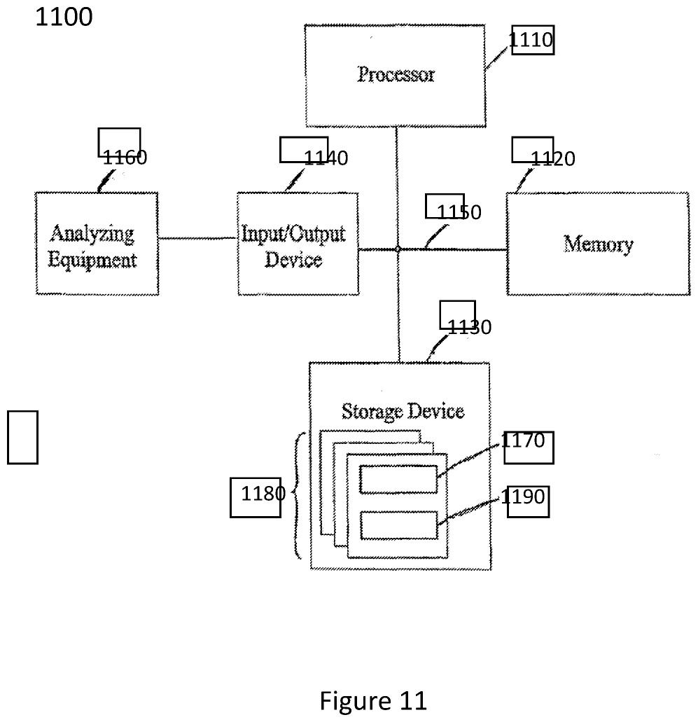

FIG. 11 is a block diagram of a computer system 1100 that can be used in the operations described above, according to one embodiment. The system 1100 includes a processor 1110, a memory 1120, a storage device 1130 and an input/output device 1140. Each of the components 1110, 1120, 1130 and 1140 are interconnected using a system bus 1150. The system may include analyzing equipment 1160 for determining a level of one or more ATTR Biomarkers in a sample.

The processor 1110 is capable of processing instructions for execution within the system 1100. In one embodiment, the processor 1110 is a single-threaded processor. In another embodiment, the processor 1110 is a multi-threaded processor. The processor 1110 is capable of processing instructions stored in the memory 1120 or on the storage device 1130, including for receiving or sending information through the input/output device 1140.

The memory 1120 stores information within the system 1100. In one embodiment, the memory 1120 is a computer-readable medium. In one embodiment, the memory 1120 is a volatile memory unit. In another embodiment, the memory 1120 is a non-volatile memory unit.

The storage device 1130 is capable of providing mass storage for the system 1100. In one embodiment, the storage device 1130 is a computer-readable medium.

The input/output device 1140 provides input/output operations for the system 1100. In one embodiment, the input/output device 1140 includes a keyboard and/or pointing device. In one embodiment, the input/output device 1140 includes a display unit for displaying graphical user interfaces.

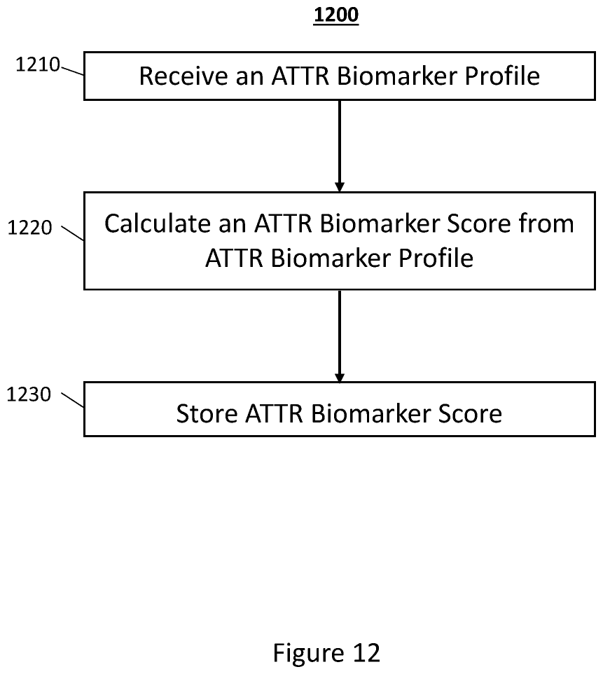

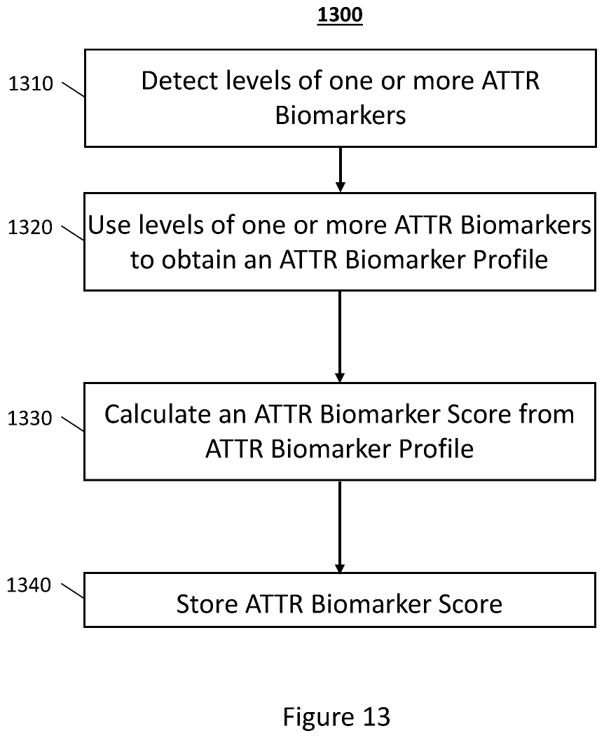

The system 1100 can be used to build a database. FIG. 12 shows a flow chart of a method 1200 for building a database for use in identifying a subject for further testing (e.g., cardiomyopathy tests), identifying a subject as being at risk or having TTR-CM, and/or identifying a subject to receive medication (e.g., a TTR stabilizer). Preferably, a method 1200 is performed in a system 1100. For example, a computer program product can include instructions that cause a processor 1110 to perform the steps of a method 1200 or a method 1300.

Method 1200 includes the following steps. Receiving, in step 1210, an subject's ATTR Biomarker Profile (e.g., levels of one or more ATTR Biomarkers in a sample). A computer program in the system 600 may include instructions for presenting a suitable graphical user interface on input/output device 640, and the graphical user interface may prompt the user to enter the levels 670 using the input/output device 640, such as a keyboard. Calculating, in step 1220, an ATTR Biomarker score from an ATTR Biomarker profile. As described herein, calculating, in step 1220, an ATTR Biomarker score from (i) an ATTR Biomarker profile and (ii) demographic factors and/or image-based biomarkers. Storing, in step 1230, an ATTR Biomarker score. The system 600 may store an ATTR Biomarker score in the storage device 630. Additionally or alternatively, a system 600 may provide a readout including an ATTR Biomarker score. A readout may also include proposed next steps for a subject and/or a confidence level associated with an ATTR Biomarker score.

Method 1300 includes the following steps. Detecting, in step 1310, levels of one or more ATTR Biomarkers in a sample, e.g., from a subject. Using, in step 1320, levels of one or more ATTR Biomarkers to obtain an ATTR Biomarker Profile. Calculating, in step 1330, an ATTR Biomarker score from an ATTR Biomarker profile. As described herein, calculating, in step 1330, an ATTR Biomarker score from (i) an ATTR Biomarker profile and (ii) demographic factors and/or image-based biomarkers. Storing, in step 1340, an ATTR Biomarker score. The system 600 may store an ATTR Biomarker score in the storage device 630. Additionally or alternatively, a system 600 may provide a readout including an ATTR Biomarker score. A readout may also include proposed next steps for a subject and/or a confidence level associated with an ATTR Biomarker score.

Additionally, non-transitory computer readable media containing executable instructions that when executed cause a processor to perform operations including a method as provided herein are provided. For example, a non-transitory computer readable medium containing executable instructions that when executed cause a processor to perform operations including a method of 1200 or 1300 described above.

### EXEMPLARY NUMBERED EMBODIMENTS

Embodiment 1. A method including detecting a level of each of two or more transthyretin amyloidosis (ATTR) Biomarkers in a sample, wherein the two or more ATTR Biomarkers include: (i) troponin I (TnI), (ii) pyruvate kinase muscle isoform 1 (PKM1), (iii) pyruvate kinase muscle isoform 2 (PKM2), (iv) N-terminal-pro hormone B-type natriuretic peptide (NT-proBNP), (v) retinol binding protein 4 (RBP4), (vi) tissue inhibitor of metalloproteinase 2 (TIMP2), (vii) neurofilament light chain (NfL), or (viii) a combination thereof.

Embodiment 2. The method of embodiment 1, detecting a level of each of three or more ATTR Biomarkers in a sample, wherein the three or more ATTR Biomarkers include: (i) TnI, (ii) PKM1, (iii) PKM2, (iv) NT-proBNP, (v) RBP4, (vi) TIMP2, (vii) NfL, or (viii) a combination thereof.

Embodiment 3. The method of embodiment 1 or 2, detecting a level of four or more ATTR Biomarkers in a sample, wherein the four or more ATTR Biomarkers include: (i) TnI, (ii) PKM1, (iii) PKM2, (iv) NT-proBNP, (v) RBP4, (vi) TIMP2, (vii) NfL, or (viii) a combination thereof.

Embodiment 4. A method including detecting a level of two or more ATTR Biomarkers in a sample, wherein the two or more ATTR Biomarkers include TnI.

Embodiment 5. The method of embodiment 4, wherein the two or more ATTR Biomarkers include TnI and PKM1.

Embodiment 6. The method of embodiment 4, wherein the two or more ATTR Biomarkers include TnI and PKM2.

Embodiment 7. The method of embodiment 4, wherein the two or more ATTR Biomarkers include TnI, PKM1, and PKM2.

Embodiment 8. The method of any one of embodiments 4-7, wherein the two or more ATTR Biomarkers further include NT-proBNP, RBP4, or both.

Embodiment 9. The method of any one of embodiments 4-8, wherein the two or more ATTR Biomarkers further include TIMP2, NfL, or both.

Embodiment 10. A method including detecting a level of two or more ATTR Biomarkers in a sample, wherein the two or more ATTR Biomarkers include NT-proBNP.

Embodiment 11. The method of any one of embodiments 4-7, wherein the two or more ATTR Biomarkers further include TnI, PKM1, PKM2, RBP4, or a combination thereof.

Embodiment 12. The method of any one of embodiments 4-8, wherein the two or more ATTR Biomarkers further include TIMP2, NfL, or both.

Embodiment 13. A method including:
(a) detecting a level of two or more ATTR Biomarkers in a sample to obtain an ATTR Biomarker profile; and
(b) using the ATTR Biomarker profile to compute a ATTR Biomarker score.

Embodiment 14. The method of embodiment 13, wherein the two or more ATTR Biomarkers include: (i) TnI, (ii) PKM1, (iii) PKM2, (iv) NT-proBNP, (v) RBP4, (vi) TIMP2, (vii) NfL, or (viii) a combination thereof.

Embodiment 15. The method of embodiment 13 or 14, detecting a level of three or more ATTR Biomarkers in a sample, wherein the three or more ATTR Biomarkers include: (i) TnI, (ii) PKM1, (iii) PKM2, (iv) NT-proBNP, (v) RBP4, (vi) TIMP2, (vii) NfL, or (viii) a combination thereof.

Embodiment 16. The method of any one of embodiments 13-15, detecting a level of four or more ATTR Biomarkers in a sample, wherein the four or more ATTR Biomarkers include: (i) TnI, (ii) PKM1, (iii) PKM2, (iv) NT-proBNP, (v) RBP4, (vi) TIMP2, (vii) NfL, or (viii) a combination thereof.

Embodiment 17. The method of any one of embodiments 13-16, wherein the two or more ATTR Biomarkers include TnI.

Embodiment 18. The method of any one of embodiments 13-17, wherein the two or more ATTR Biomarkers include TnI and PKM1.

Embodiment 19. The method of any one of embodiments 13-18, wherein the two or more ATTR Biomarkers include TnI and PKM2.

Embodiment 20. The method of any one of embodiments 13-19, wherein the two or more ATTR Biomarkers include TnI, PKM1, and PKM2.

Embodiment 21. The method of any one of embodiments 17-20, wherein the two or more ATTR Biomarkers further include NT-proBNP, RBP4, or both.

Embodiment 22. The method of any one of embodiments 17-21, wherein the two or more ATTR Biomarkers further include TIMP2, NfL, or both.

Embodiment 23. The method of any one of embodiments 13-16, wherein the two or more ATTR Biomarkers include NT-proBNP.

Embodiment 24. The method of embodiment 23, wherein the two or more ATTR Biomarkers further include TnI, PKM1, PKM2, RBP4, or a combination thereof.

Embodiment 25. The method of embodiment 23 or 24, wherein the two or more ATTR Biomarkers further include TIMP2, NfL, or both.

Embodiment 26. The method of embodiment 1, wherein the two or more ATTR Biomarkers include or consist of TnI, PKM1, NT-proBNP, and RBP4.

Embodiment 27. The method of embodiment 1, wherein the two or more ATTR Biomarkers include or consist of TnI, PKM1, and RBP4.

Embodiment 28. The method of embodiment 1, wherein the two or more ATTR Biomarkers include or consist of TnI, PKM1, and NT-proBNP.

Embodiment 29. The method of embodiment 1, wherein the two or more ATTR Biomarkers include or consist of TnI, PKM2, NT-proBNP, and RBP4.

Embodiment 30. The method of embodiment 1, wherein the two or more ATTR Biomarkers include or consist of TnI, PKM2, and RBP4.

Embodiment 31. The method of embodiment 1, wherein the two or more ATTR Biomarkers include or consist of TnI, PKM2, and NT-proBNP.

Embodiment 32. The method of embodiment 1, wherein the two or more ATTR Biomarkers include or consist of TnI, PKM1, PKM2, NT-proBNP, and RBP4.

Embodiment 33. The method of embodiment 1, wherein the two or more ATTR Biomarkers include or consist of TnI, PKM1, PKM2, and RBP4.

Embodiment 34. The method of embodiment 1, wherein the two or more ATTR Biomarkers include or consist of TnI, NT-proBNP, and RBP4.

Embodiment 35. The method of embodiment 1, wherein the two or more ATTR Biomarkers include or consist of TnI, NT-proBNP, and TIMP2.

Embodiment 36. The method of embodiment 1, wherein the two or more ATTR Biomarkers include or consist of TnI and NT-proBNP.

Embodiment 37. The method of embodiment 1, wherein the two or more ATTR Biomarkers include or consist of TnI and RBP4.

Embodiment 38. The method of embodiment 1, wherein the two or more ATTR Biomarkers include or consist of TnI, RBP4 and TIMP2.

Embodiment 39. The method of embodiment 1, wherein the two or more ATTR Biomarkers include or consist of TnI and TIMP2.

Embodiment 40. The method of any one of embodiments 1-3, 4, 6, 8, 9, 10-12, 13-17, 19, and 21-25, wherein the two or more ATTR Biomarkers do not include PKM1.

Embodiment 41. The method of any one of embodiments 1-3, 4, 5, 8, 9, 10-12, 13-18, and 21-25, wherein the two or more ATTR Biomarkers do not include PKM2.

Embodiment 42. The method of any one of embodiments 1-3, 4, 8, 9, 10-12, 13-17, and 21-25, wherein the two or more ATTR Biomarkers do not include either PKM1 or PKM2.

Embodiment 43. The method of any one of embodiments 1-42, wherein the two or more ATTR Biomarkers do not include SMOC-2.

Embodiment 44. The method of any one of embodiments 1-42, wherein the two or more ATTR Biomarkers do not include DCN.

Embodiment 45. The method of any one of embodiments 1-42, wherein the two or more ATTR Biomarkers do not include either SMOC-2 or DCN.

Embodiment 46. The method of any one of embodiments 1-45, wherein the method is a method of determining a subject's risk of developing amyloid transthyretin cardiomyopathy (TTR-CM), and the sample was obtained from the subject.

Embodiment 47. The method of any one of embodiments 1-45, wherein the method is a method of diagnosing a subject with TTR-CM, and the sample was obtained from the subject.

Embodiment 48. The method of any one of embodiments 1-45, wherein the method is a method of treating TTR-CM in a subject at risk of or suffering from TTR-CM.

Embodiment 49. The method of any one of embodiments 46-48, wherein TTR-CM results from wild-type transthyretin amyloidosis (ATTRwt).

Embodiment 50. The method of any one of embodiments 46-49, wherein the subject tests negative for familial amyloid cardiomyopathy (ATTRm) by genetic testing.

Embodiment 51. The method of any one of embodiments 1-45, wherein the method is a method of selecting a subject to receive one or more doses of a TTR stabilizing agent, and the sample was obtained from the subject.

Embodiment 52. The method of embodiment 51, wherein the method further includes administering to the subject one or more doses a TTR stabilizing agent.

Embodiment 53. The method of any one of embodiments 1-45, wherein the method is a method of selecting a subject for one or more cardiomyopathy tests, and the sample was obtained from the subject.

Embodiment 54. The method of embodiment 53, wherein the one or more cardiomyopathy tests include an echocardiogram, an advanced imaging method, or both.

Embodiment 55. The method of embodiment 54, wherein the advanced imaging method includes cardiac magnetic resonance imaging (CMR), scintigraphy, or both.

Embodiment 56. The method of embodiment 55, wherein scintigraphy includes use of a radioisotope conjugate such as 977 Tc-Pyrophosphate.

Embodiment 57. The method of embodiment 55 or 56, wherein scintigraphy is performed using single photon emission computed tomography (SPECT).

Embodiment 58. The method of any one of embodiments 1-45, wherein the method is a method of determining a patient does not have or is not at risk of developing TTR-CM.

Embodiment 59. The method of any one of embodiments 1-57, wherein the sample is or includes a biological sample.

Embodiment 60. The method of embodiment 59, wherein the biological sample includes blood, serum, plasma, or cardiac tissue.

Embodiment 61. The method of any one of embodiments 1-60, wherein the sample was obtained from a subject.

Embodiment 62. The method of embodiment 61, wherein the subject is a human.

Embodiment 63. The method of embodiment 61 or 62, wherein the subject is suffering from or is at risk of developing amyloid transthyretin cardiomyopathy (TTR-CM).

Embodiment 64. The method of any one of embodiments 46-63, wherein the subject is at least 65 years old.

Embodiment 65. The method of any one of embodiments 1-64, wherein the step of detecting a level of two or more ATTR Biomarkers in the sample includes detecting the presence, absence, level or genotype of each of the two or more ATTR Biomarkers in the sample.

Embodiment 66. The method of any one of embodiments 1-65, wherein the step of detecting a level of two or more ATTR Biomarkers in the sample includes performing mass spectrometry.

Embodiment 67. The method of any one of embodiments 1-66, wherein the step of detecting a level of two or more ATTR Biomarkers in the sample includes measuring chemiluminescence.

Embodiment 68. The method of any one of embodiments 1-67, wherein the step of detecting a level of two or more ATTR Biomarkers in the sample includes detecting two or more nucleotides that encode the two or more ATTR Biomarkers in the sample.

Embodiment 69. The method of embodiment 68, wherein detecting two or more nucleotides that encode the two or more ATTR Biomarkers includes performing a nucleic acid amplification method.

Embodiment 70. The method of embodiment 69, wherein the nucleic acid amplification method is selected from the group consisting of polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA).

Embodiment 71. The method of any one of embodiments 68-70, wherein detecting two or more nucleotides that encode the two or more ATTR Biomarkers includes detecting hybridization between two or more nucleic acid probes and the two or more nucleotides that encode the two or more ATTR Biomarkers.

Embodiment; 62 The method of embodiment 71, wherein the two or more nucleic acid probes are each complementary to at least a portion of one of the two or more nucleotides that encode the two or more ATTR Biomarkers.$

Embodiment 73. The method of any one of embodiments 68-72, wherein the nucleotides that encode the two or more ATTR Biomarkers are DNA.

Embodiment 74. The method of embodiment 73, wherein the DNA is cDNA.

Embodiment 75. The method of any one of embodiments 68-72, wherein the nucleotides that encode the two or more ATTR Biomarkers are RNA.

Embodiment 76. The method of any one of embodiments 1-75, wherein the step of detecting a level of the two or more ATTR Biomarkers in the sample includes performing an immunoassay.

Embodiment 77. The method of embodiment 76, wherein the immunoassay is a chemiluminescent immunoassay.

Embodiment 78. The method of embodiment 76 or 77, wherein the immunoassay is selected from the group consisting of immunoprecipitation; Western blot; ELISA; immunohistochemistry; immunocytochemistry; flow cytometry; and, immuno-PCR.

Embodiment 79. The method of any one of embodiments 76-78, wherein the immunoassay is an ELISA.

Embodiment 80. The method of any one of embodiments 76-79, wherein the immunoassay is a high-throughput and/or automated immunoassay platform.

Embodiment 81. The method of any one of embodiments 1-80, wherein detecting a level of the two or more ATTR Biomarkers in the sample includes contacting the sample with two or more anti-ATTR Biomarker antibody agents.

Embodiment 82. The method of any one of embodiments 1-81, wherein detecting a level of the two or more ATTR Biomarkers in a sample obtained from the subject includes contacting the sample with two or more detection agents.

Embodiment 83. The method of embodiment 82, wherein the two or more anti-ATTR Biomarker antibody agents are labeled with the two or more detection agents.

Embodiment 84. The method of embodiment 82 or 83, wherein the two or more detection agents are two or more acridinium ester molecules.

Embodiment 85. The method of any one of embodiments 1-84, wherein detecting a level of the two or more ATTR Biomarkers in the sample includes detecting binding between the two or more ATTR Biomarkers and the two or more anti-ATTR Biomarker antibody agents.

Embodiment 86. The method of embodiment 85, wherein detecting binding between the two or more ATTR Biomarkers and the two or more anti-ATTR Biomarker antibody agents includes performing immunocytochemistry (ICC).

Embodiment 87. The method of embodiment 85 or 86, wherein detecting binding between the two or more ATTR Biomarkers and the two or more anti-ATTR Biomarker antibody agents includes determining at least one ATTR Biomarker absorbance value or emission value.

Embodiment 88. The method of embodiment 1-87, wherein detecting a level of two or more ATTR Biomarkers in the sample includes detecting a level of each of the two or more ATTR Biomarkers, wherein the method further includes comparing a level of each of the two or more ATTR Biomarkers to a threshold value for the respective ATTR Biomarkers, and wherein the level of each of the one or more ATTR Biomarkers that is detected is above the threshold value for the respective ATTR Biomarkers.

Embodiment 89. The method of embodiment 88, further including determining the subject is suffering from or is at risk of developing TTR-CM if the level of each of the one or more ATTR Biomarkers is above a threshold value for the respective ATTR Biomarkers.

Embodiment 90. The method of embodiment 88 or 89, further including diagnosing the subject with TTR-CM if the level of each of the one or more ATTR Biomarkers is above a threshold value for the respective ATTR Biomarkers.

Embodiment 91. The method of any one of embodiments 88-90, further including recommending the subject for one or more cardiomyopathy tests if the level of each of the one or more ATTR Biomarkers is above a threshold value for the respective ATTR Biomarkers.

Embodiment 92. The method of any one of embodiments 88-91, wherein the level of each of the one or more ATTR Biomarkers is at least 1.3, at least 1.4, at least 1.5, at least 1.6, at least 1.7, at least 1.8, or at least 1.9 fold greater than a threshold value for the respective ATTR Biomarkers.

Embodiment 93. The method of any one of embodiments 88-92, wherein the threshold value for an ATTR Biomarker is an average of values for the ATTR Biomarker detected for two or more control samples.

Embodiment 94. The method of embodiment 93, wherein the two or more control samples are each a sample obtained from a subject who does not have ATTR amyloidosis.

Embodiment 95. The method of any one of embodiments 88-92, wherein the threshold value for an ATTR Biomarker is a value reported in a standard table.

Embodiment 96. The method of any one of embodiments 88-92, further including recommending the subject for cardiac biopsy if the level of each of the one or more ATTR Biomarkers is above a threshold value and the subject tests positive for cardiomyopathy in one or more of the cardiomyopathy tests.

Embodiment 97. The method of any one of embodiments 13-87, wherein using the ATTR Biomarker profile to compute an ATTR Biomarker score includes applying an algorithm to the ATTR Biomarker profile to compute an ATTR Biomarker score, wherein the algorithm is a decision tree algorithm, a neural boosted algorithm, a bootstrap forest algorithm, a boosted tree algorithm, a K nearest neighbors algorithm, a generalized regression forward selection algorithm, a generalized regression pruned forward selection algorithm, a fit stepwise algorithm, a generalized regression lasso algorithm, a generalized regression elastic net algorithm, a generalized regression ridge algorithm, a nominal logistic algorithm, a support vector machines algorithm, a discriminant algorithm, or a naïve Bayes algorithm.

Embodiment 98. The method of any one of embodiments 13-87 and 97, wherein using the ATTR Biomarker profile to compute an ATTR Biomarker score includes applying an algorithm to the ATTR Biomarker profile to compute an ATTR Biomarker score, wherein the algorithm is a decision tree algorithm, a neural boosted algorithm, a bootstrap forest algorithm, a boosted tree algorithm, a generalized regression lasso algorithm, a generalized regression elastic net algorithm, a generalized regression ridge algorithm, a nominal logistic algorithm, a support vector machines algorithm, or a discriminant algorithm.

Embodiment 99. The method of any one of embodiments 13-87, 97, and 98, wherein using the ATTR Biomarker profile to compute an ATTR Biomarker score includes applying an algorithm to the ATTR Biomarker profile to compute an ATTR Biomarker score, wherein the algorithm is a decision tree algorithm, a neural boosted algorithm, a bootstrap forest algorithm, a boosted tree algorithm, or a support vector machines algorithm.

Embodiment 100. The method of any one of embodiments 13-87 and 97-99, further including using the ATTR Biomarker score to determine if a subject from which the sample was obtained is at risk of or suffering from TTR-CM.

Embodiment 101. The method of any one of embodiments 13-87 and 97-100, further including using the ATTR Biomarker score to determine if a subject from which the sample was obtained is selected for one or more cardiomyopathy tests.

Embodiment 102. The method of any one of embodiments 13-87 and 97-101, further including using the ATTR Biomarker score to determine if a subject from which the sample was obtained is selected to receive one or more doses of a TTR stabilizing agent.

Embodiment 103. The method of any one of embodiments 1-102, further including immunochemical staining of biopsy tissue from the subject.

Embodiment 104. The method of embodiment 88, wherein the biopsy tissue includes cardiac biopsy tissue.

Embodiment 105. The method of embodiment 88 or 89, wherein immunochemical staining includes use of two or more antibody agents to kappa or lambda light chain amyloid deposits in heart tissue and/or two or more antibody agents to transthyretin deposits.

Embodiment 106. The method of any one of embodiments 88-90, further including diagnosing the subject with TTR-CM if immunochemical staining indicates presence of transthyretin deposits in the cardiac tissue.

Embodiment 107. A method including:
(a) detecting a level of two or more ATTR Biomarkers in a sample obtained from a subject to obtain an ATTR Biomarker profile; and
(b) using the ATTR Biomarker profile to compute a ATTR Biomarker score,
wherein the two or more ATTR Biomarkers include: (i) TnI, (ii) PKM1, (iii) PKM2, (iv) NT-proBNP, (v) RBP4, (vi) TIMP2, (vii) NfL, or (viii) a combination thereof, and
wherein using the ATTR Biomarker profile to compute an ATTR Biomarker score includes applying an algorithm to the ATTR Biomarker profile to compute an ATTR Biomarker score, wherein the algorithm is a decision tree algorithm, a neural boosted algorithm, a bootstrap forest algorithm, a boosted tree algorithm, or a support vector machines algorithm.

Embodiment 108. The method of embodiment 107, wherein the subject is a human subject.

Embodiment 109. The method of embodiment 107 or 108, wherein the subject is at least 65 years old.

Embodiment 110. The method of any one of embodiments 107-109, wherein the sample includes blood, serum, plasma, or cardiac tissue.

Embodiment 111. The method of any one of embodiments 107-110, further including using the ATTR Biomarker score to determine if a subject from which the sample was obtained is at risk of or suffering from TTR-CM.

Embodiment 112. The method of any one of embodiments 107-111, further including using the ATTR Biomarker score to determine a subject's risk of developing amyloid transthyretin cardiomyopathy (TTR-CM).

Embodiment 113. The method of any one of embodiments 107-112, further including using the ATTR Biomarker score to diagnose a subject with TTR-CM.

Embodiment 114. The method of embodiment 113, wherein TTR-CM results from wild-type transthyretin amyloidosis (ATTRwt).

Embodiment 115. The method of any one of embodiments 107-114, further including using the ATTR Biomarker score to determine if a subject from which the sample was obtained is selected for one or more cardiomyopathy tests.

Embodiment 116. The method of embodiment 115, wherein the one or more cardiomyopathy tests include an echocardiogram, an advanced imaging method, or both.

Embodiment 117. The method of embodiment 116, wherein the advanced imaging method includes cardiac magnetic resonance imaging (CMR), scintigraphy, or both.

Embodiment 118. The method of embodiment 117, wherein scintigraphy includes use of a radioisotope conjugate such as 99m Tc-Pyrophosphate.

Embodiment 119. The method of embodiment 117 or 118, wherein scintigraphy is performed using single photon emission computed tomography (SPECT).

Embodiment 120. The method of any one of embodiments 107-119, further including using the ATTR Biomarker score to determine if a subject from which the sample was obtained is selected to receive one or more doses of a TTR stabilizing agent.

Embodiment 121. The method of embodiment 120, wherein the method further includes administering to the subject one or more doses a TTR stabilizing agent.

Embodiment 122. A non-transitory computer readable medium containing executable instructions that when executed cause a processor to perform operations including the method of any one of embodiments 1-121.

Embodiment 123. A composition including:
(a) one or more ATTR Biomarkers, wherein the one or more ATTR Biomarkers include: (i) TnI, (ii) PKM1, (iii) PKM2, (iv) NT-proBNP, (v) RBP4, (vi) TIMP2, (vii) NfL, or (viii) a combination thereof; and
(b) one or more anti-ATTR Biomarker agents, wherein the one or more anti-ATTR Biomarker agents include: (i) an anti-TnI agent, (ii) an anti-PKM1 agent, (iii) an anti-PKM2 agent, (iv) an anti-NT-proBNP agent, (v) an anti-RBP4 agent, (vi) an anti-TIMP2 agent, (vii) an anti-NfL agent, or (viii) a combination thereof.

Embodiment 124. A composition including:
(a) one or more ATTR Biomarkers, wherein the one or more ATTR Biomarkers include TnI, and
(b) one or more anti-ATTR Biomarker agents, wherein the one or more anti-ATTR Biomarker agents include an anti-TnI agent.

Embodiment 125. The composition of embodiment 124, wherein:
(a) the one or more ATTR Biomarkers include TnI and PKM1, and
(b) the one or more anti-ATTR Biomarker agents include an anti-TnI agent and an anti-PKM1 agent.

Embodiment 126. The composition of embodiment 124, wherein:
(a) the one or more ATTR Biomarkers include TnI and PKM2, and
(b) the one or more anti-ATTR Biomarker agents include an anti-TnI agent and an anti-PKM2 agent.

Embodiment 127. The composition of embodiment 124, wherein:
(a) the one or more ATTR Biomarkers include TnI, PKM1, and PKM2, and
(b) the one or more anti-ATTR Biomarker agents include an anti-TnI agent, an anti-PKM1 agent, and an anti-PKM2 agent.

Embodiment 128. The composition of any one of embodiments 123-127, wherein:
(a) the one or more ATTR Biomarkers further include NT-proBNP, RBP4, or both, and
(b) the one or more anti-ATTR Biomarker agents further include an anti-NT-proBNP agent, an anti-RBP4 agent, or both.

Embodiment 129. The composition of any one of embodiments 123-128, wherein:
(a) the one or more ATTR Biomarkers further include TIMP2, NfL, or both, and (b) the one or more anti-ATTR Biomarker agents further include an anti-TIMP2 agent, an anti-NfL agent, or both.

Embodiment 130. A composition including:
(a) one or more ATTR Biomarkers, wherein the one or more ATTR Biomarkers include NT-proBNP, and
(b) one or more anti-ATTR Biomarker agents, wherein the one or more anti-ATTR Biomarker agents include an anti-NT-proBNP agent.

Embodiment 131. The composition of embodiment 130, wherein:
(a) the one or more ATTR Biomarkers further include: (i) TnI, (ii) PKM1, (iii) PKM2, (iv) RBP4, (v) TIMP2, (vi) NfL, or (vii) a combination thereof; and
(b) the one or more anti-ATTR Biomarker agents further include: (i) an anti-TnI agent, (ii) an anti-PKM1 agent, (iii) an anti-PKM2 agent, (iv) an anti-RBP4 agent, (v) an anti-TIMP2 agent, (vi) an anti-NfL agent, or (vii) a combination thereof.

Embodiment 132. The composition of embodiment 130 or 131, wherein:
(a) the one or more ATTR Biomarkers include NT proBNP, TnI, and RBP, and
(b) the one or more anti-ATTR Biomarker agents include an anti-NT proBNP agent, an anti-TnI agent, and an anti-RBP agent.

Embodiment 133. The composition of any one of embodiments 123, 124, 126, and 128-132, wherein the one or more ATTR Biomarkers do not include PKM1 and the one or more anti-ATTR Biomarker agents do not include an anti-PKM1 agent.

Embodiment 134. The composition of any one of embodiments 123-125, and 128-132, wherein the one or more ATTR Biomarkers do not include PKM2 and the one or more anti-ATTR Biomarker agents do not include an anti-PKM2 agent.

Embodiment 135. The composition of any one of embodiments 123, 124, and 128-132, wherein the one or more ATTR Biomarkers do not include either PKM1 or PKM2, and the one or more anti-ATTR Biomarker agents do not include either an anti-PKM1 agent or an anti-PKM2 agent.

Embodiment 136. A kit for detecting TTR-CM, said kit including:
(a) one or more anti-ATTR Biomarker agents, wherein the one or more anti-ATTR Biomarker agents include: (i) an anti-TnI agent, (ii) an anti-PKM1 agent, (iii) an anti-PKM2 agent, (iv) an anti-NT-proBNP agent, (v) an anti-RBP4 agent, (vi) an anti-TIMP2 agent, (vii) an anti-NfL agent, or (viii) a combination thereof, and (b) instructions for use.

Embodiment 137. A kit for detecting TTR-CM, said kit including:
(a) one or more anti-ATTR Biomarker agents, wherein the one or more anti-ATTR Biomarker agents include an anti-TnI agent; and
(b) instructions for use.

Embodiment 138. The kit of embodiment 137, wherein the one or more anti-ATTR Biomarker agents include an anti-TnI agent and an anti-PKM1 agent.

Embodiment 139. The kit of embodiment 137, wherein the one or more anti-ATTR Biomarker agents include an anti-TnI agent and an anti-PKM2 agent.

Embodiment 140. The kit of embodiment 137, wherein the one or more anti-ATTR Biomarker agents include an anti-TnI agent, an anti-PKM1 agent, and an anti-PKM2 agent.

Embodiment 141. The kit of any one of embodiments 137-140, wherein the one or more anti-ATTR Biomarker agents further include an anti-NT-proBNP agent, an anti-RBP4 agent, or both.

Embodiment 142. The kit of any one of embodiments 137-141, wherein the one or more anti-ATTR Biomarker agents further include an anti-TIMP2 agent, an anti-NfL agent, or both.

Embodiment 143. A kit including:
(a) one or more anti-ATTR Biomarker agents, wherein the one or more anti-ATTR Biomarker agents include an anti-NT-proBNP agent; and
(b) instructions for use.

Embodiment 144. The kit of embodiment 143, wherein the one or more anti-ATTR Biomarker agents further include: (i) an anti-TnI agent, (ii) an anti-PKM1 agent, (iii) an anti-PKM2 agent, (iv) an anti-RBP4 agent, (v) an anti-TIMP2 agent, (vi) an anti-NfL agent, or (vii) a combination thereof.

Embodiment 145. The kit of embodiment 143 or 144, wherein the one or more anti-ATTR Biomarker agents include an anti-NT-proBNP agent, an anti-TnI agent, and an anti-RBP agent.

Embodiment 146. The method of any one of embodiments 136, 137, 139, and 141-145, wherein the one or more ATTR Biomarkers do not include PKM1 and the one or more anti-ATTR Biomarker agents do not include an anti-PKM1 agent.

Embodiment 147. The method of any one of embodiments 136-138 and 141-145, wherein the one or more ATTR Biomarkers do not include PKM2 and the one or more anti-ATTR Biomarker agents do not include an anti-PKM2 agent.

Embodiment 148. The method of any one of embodiments 136, 137, and 141-145, wherein the one or more ATTR Biomarkers do not include either PKM1 or PKM2, and the one or more anti-ATTR Biomarker agents do not include either an anti-PKM1 agent or an anti-PKM2 agent.

Embodiment 149. The kit of any one of embodiments 136-148, wherein the kit further includes a TTR stabilizing agent.

Embodiment 150. The kit of any one of embodiments 136-149, wherein the one or more anti-ATTR Biomarker agents include one or more antibody agents.

Embodiment 151. The kit of any one of embodiments 136-150 wherein the one or more anti-ATTR Biomarker agents include one or more nucleic acid probes.

Embodiment 152. The kit of embodiment 150 or 151, wherein one or more of the antibody agents are labeled with a detectable moiety.

Embodiment 153. The kit of embodiments 150-152, further including one or more secondary antibody agents that specifically bind to the one or more anti-ATTR Biomarker antibody agents.

Embodiment 154. The kit of embodiment 153, wherein the one or more anti-ATTR Biomarker antibody agents and/or the one or more of the secondary antibody agents are linked to an enzyme.

Embodiment 155. The kit of embodiments 136-154, further including a detection agent.

Embodiment 156. The kit of embodiment 155, wherein the detection agent is or includes a substrate for the enzyme.

Embodiment 157. The kit of embodiment 155, wherein the detection agent is or includes one or more acridinium ester molecules.

Embodiment 158. The kit of embodiment 153, wherein the one or more anti-ATTR Biomarker antibody agents and/or the one or more of the secondary antibody agents are labeled with one or more of the acridinium ester molecules.

Embodiment 159. The kit of any one of embodiments 151-158, wherein the one or more of anti-ATTR Biomarker nucleic acid probes are complementary to one or more nucleotides that encode an ATTR Biomarker.

Embodiment 160. The kit of any one of embodiments 151-159, wherein at least a portion of each anti-ATTR Biomarker nucleic acid probe hybridizes to one or more nucleotides that encode an ATTR Biomarker.

Embodiment 161. The kit of any one of embodiments 159 and 160, wherein the nucleotides that encode one or more ATTR Biomarkers are DNA.

Embodiment 162. The kit of embodiment 161, wherein the DNA is cDNA.

Embodiment 163. The kit of any one of embodiments 159 and 160, wherein the nucleotides that encode one or more ATTR Biomarkers are RNA.

Embodiment 164. The kit of embodiments 151-163, wherein the one or more of anti-ATTR Biomarker nucleic acid probes are labeled with one or more of the detection agents.

Embodiment 165. The kit of embodiment 164, wherein the detection agents indicate presence of nucleotides that encode one or more ATTR Biomarkers.

Embodiment 166. The kit of embodiments 136-165, further including one or more control samples.

Embodiment 167. The kit of embodiment 166, wherein the control samples include one or more ATTR Biomarker standards.

Embodiment 168. The kit of embodiment 167, wherein the one or more ATTR Biomarker standards include a recombinant ATTR Biomarker.

Embodiment 169. The kit of embodiment 167, wherein the one or more ATTR Biomarker standards include synthetic ATTR Biomarker nucleic acids.

Embodiment 170. Use of a kit according to embodiments 136-169 in an in vitro diagnostic assay to diagnose TTR-CM in a subject.

### EXEMPLIFICATION

### Example 1: Identification of Markers for ATTR Amyloidosis

This example demonstrates a method for screening human plasma to identify biomarkers indicative of ATTR amyloidosis and TTR-CM.

Mass spectrometry (MS) was employed to identify biomarkers specific for ATTR amyloidosis. Human normal (N=4) and ATTRwt EDTA plasma (N=6) were added (50 uL) to prewashed (1×TTBS) goat-anti-mouse 96-well ELISA plates and incubated for 1 hour at room temperature. Plates were subsequently washed (three times in 1×TTBS) and residual captured material was extracted and prepared for MS.

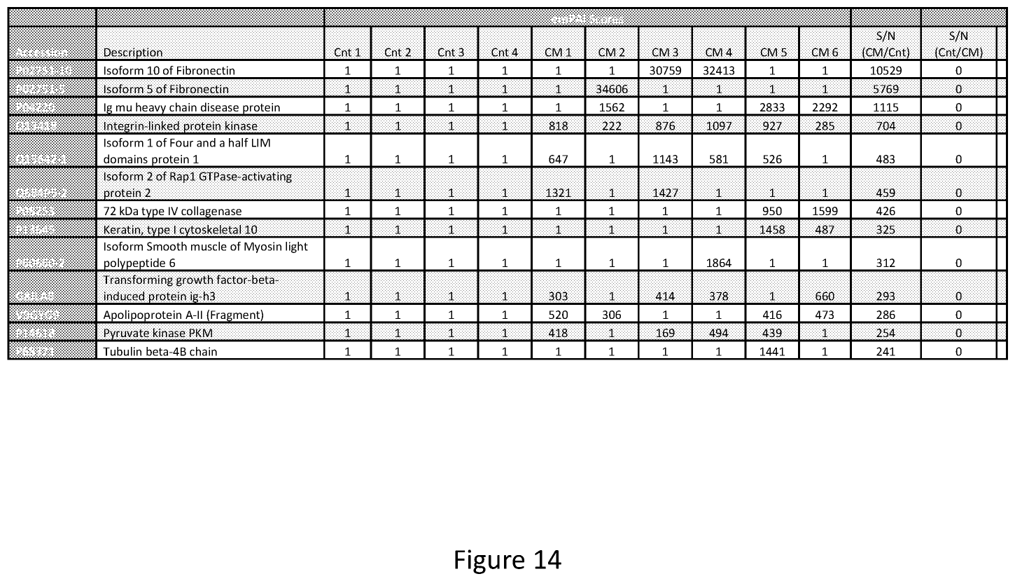

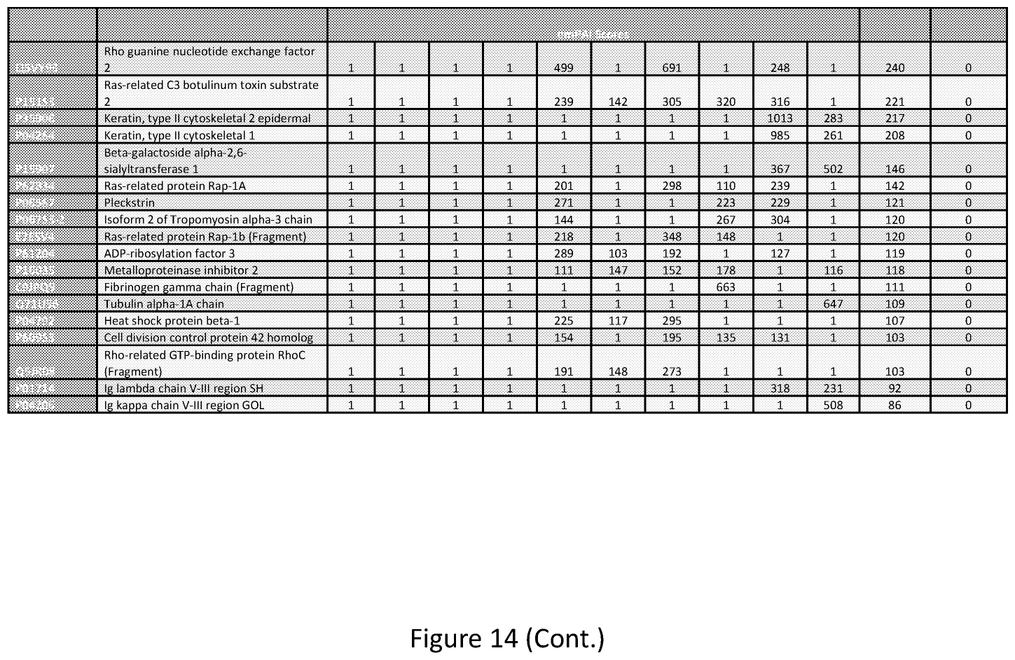

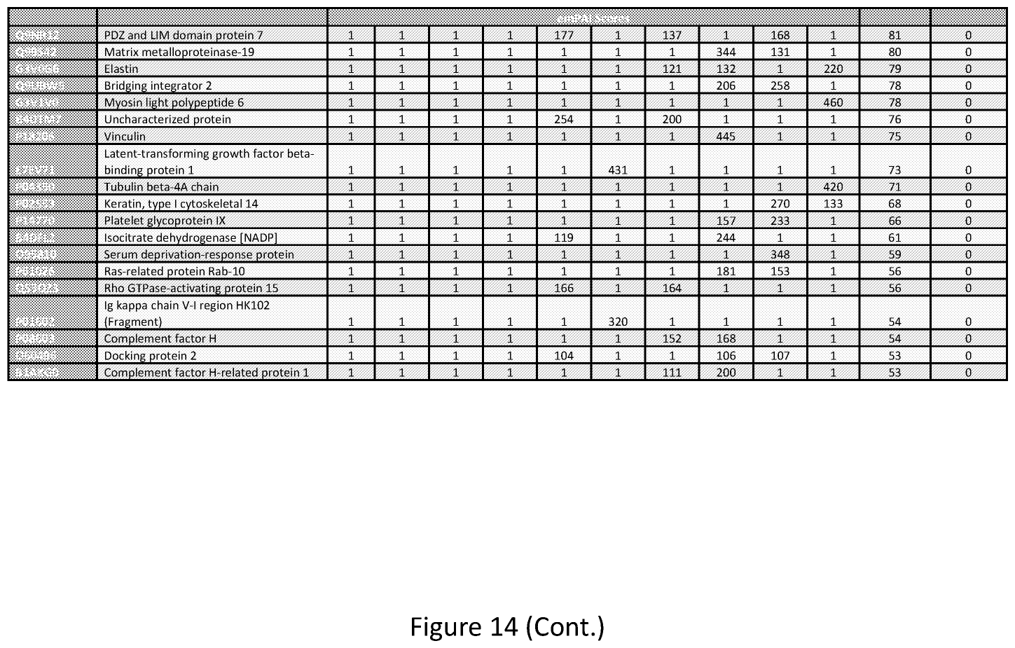

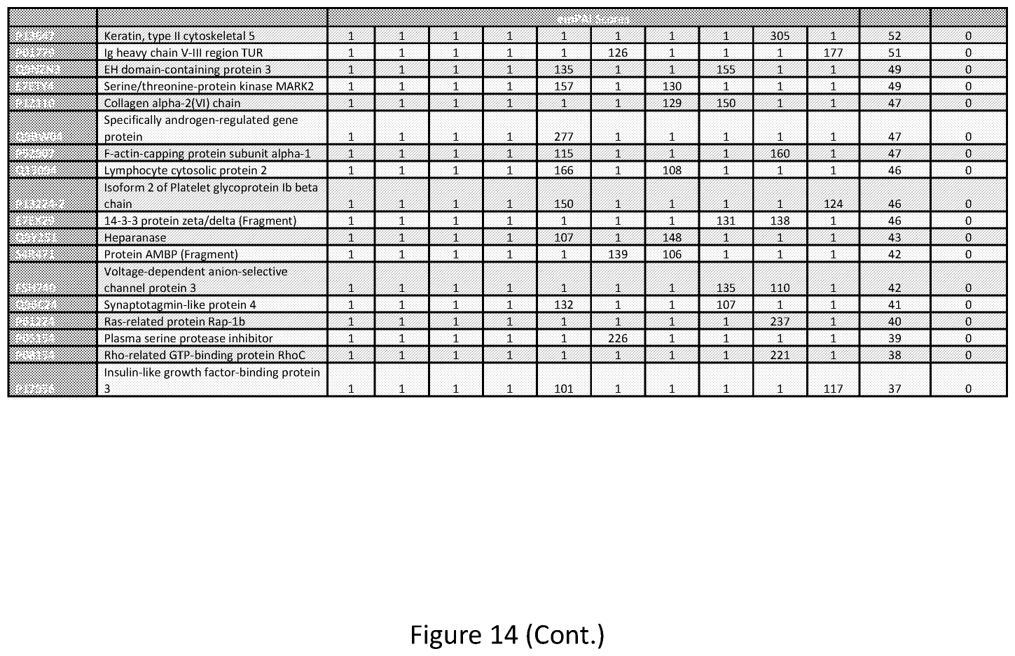

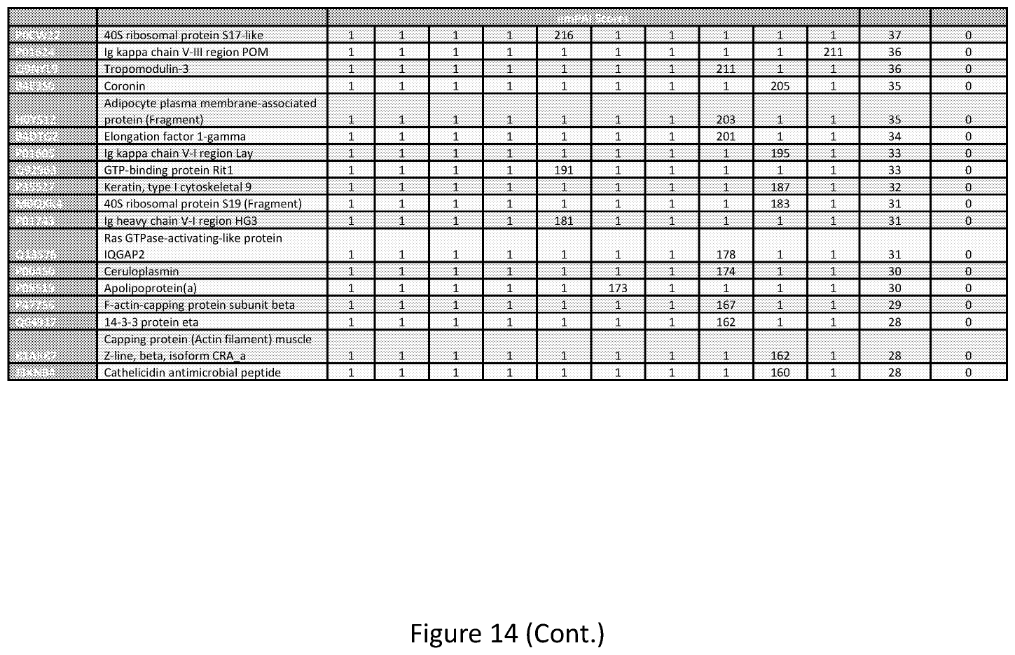

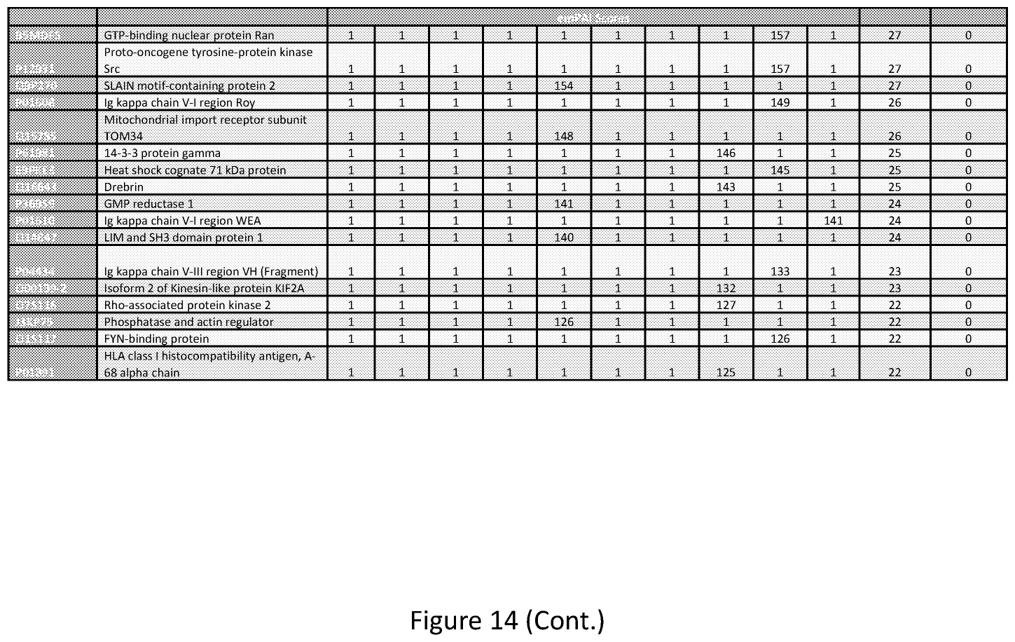

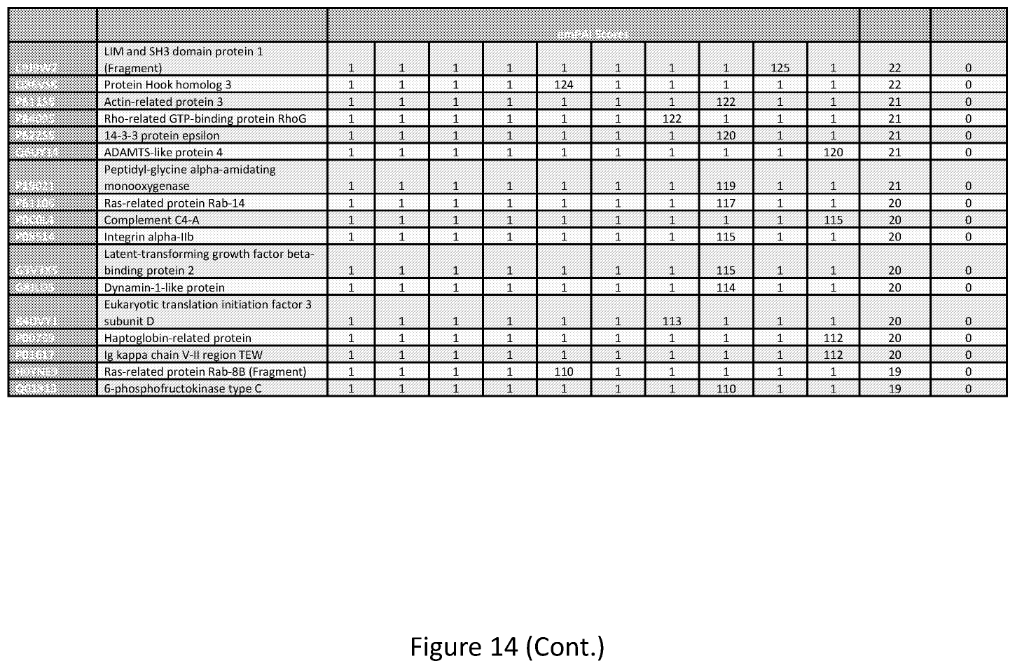

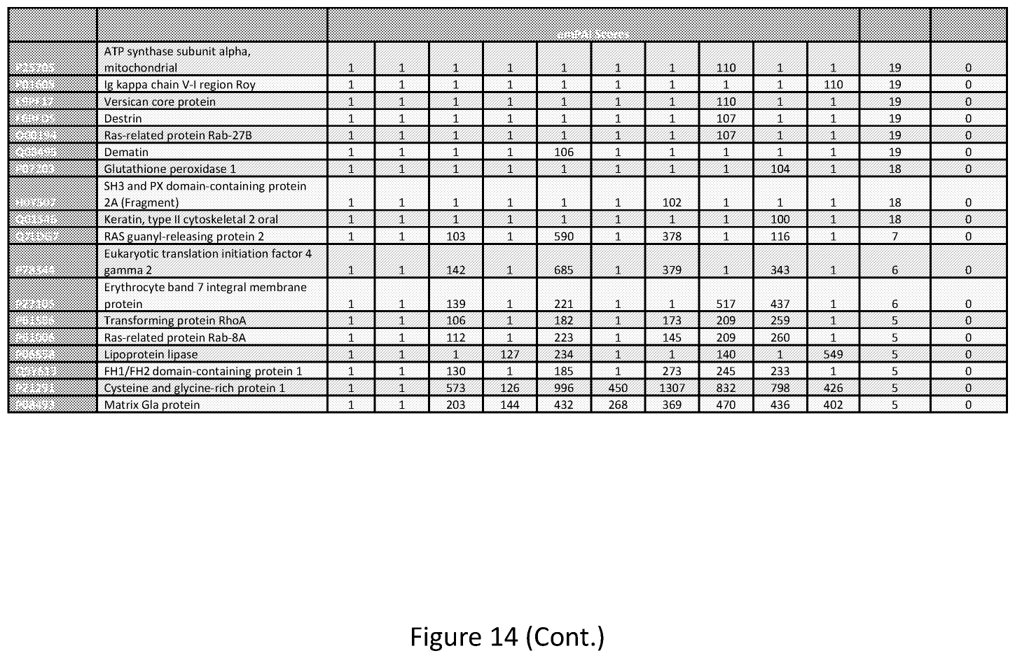

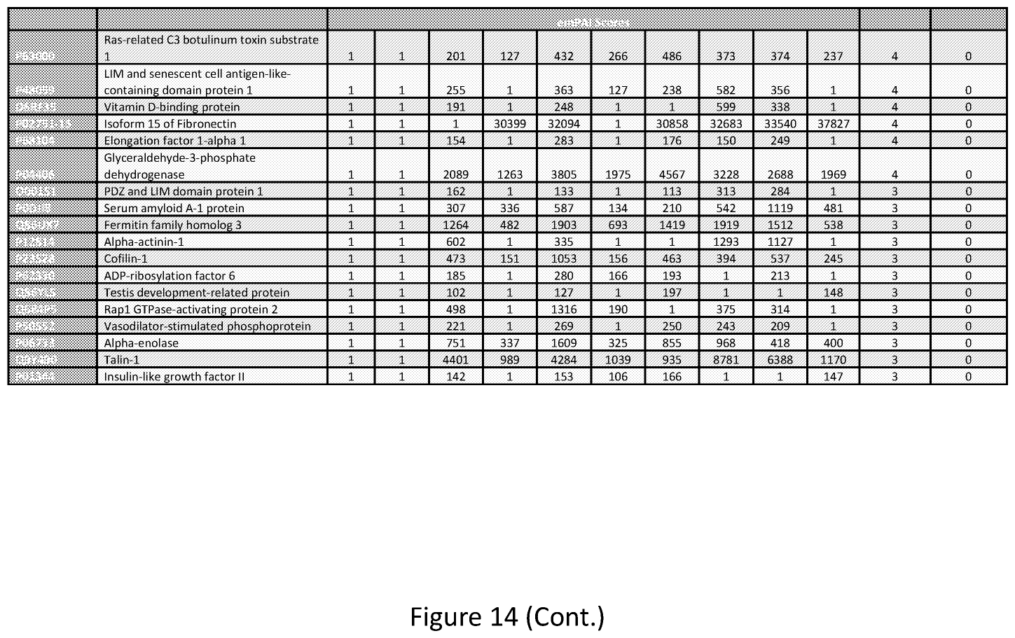

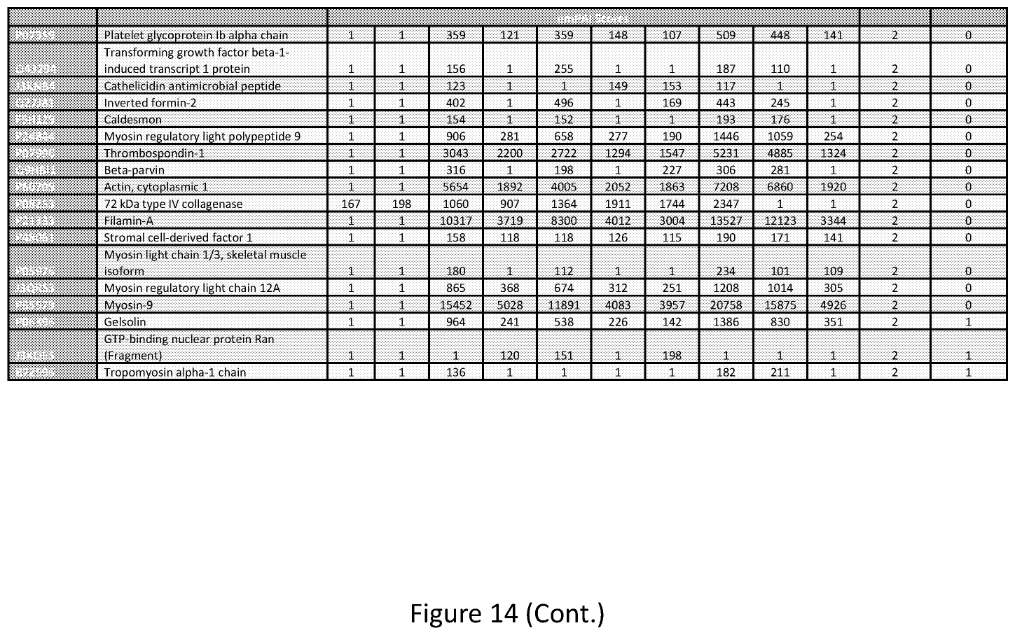

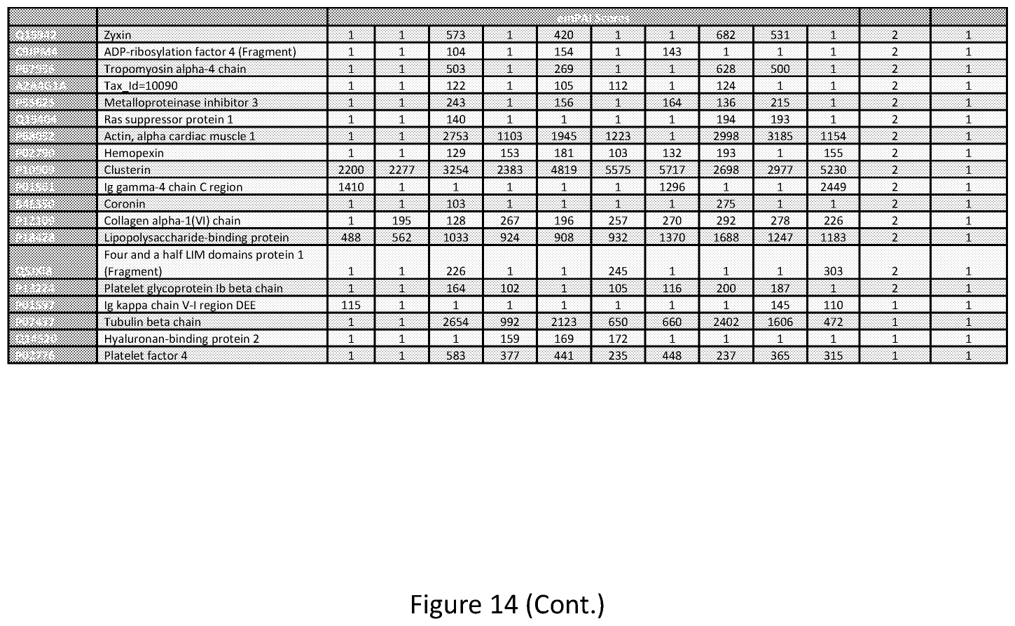

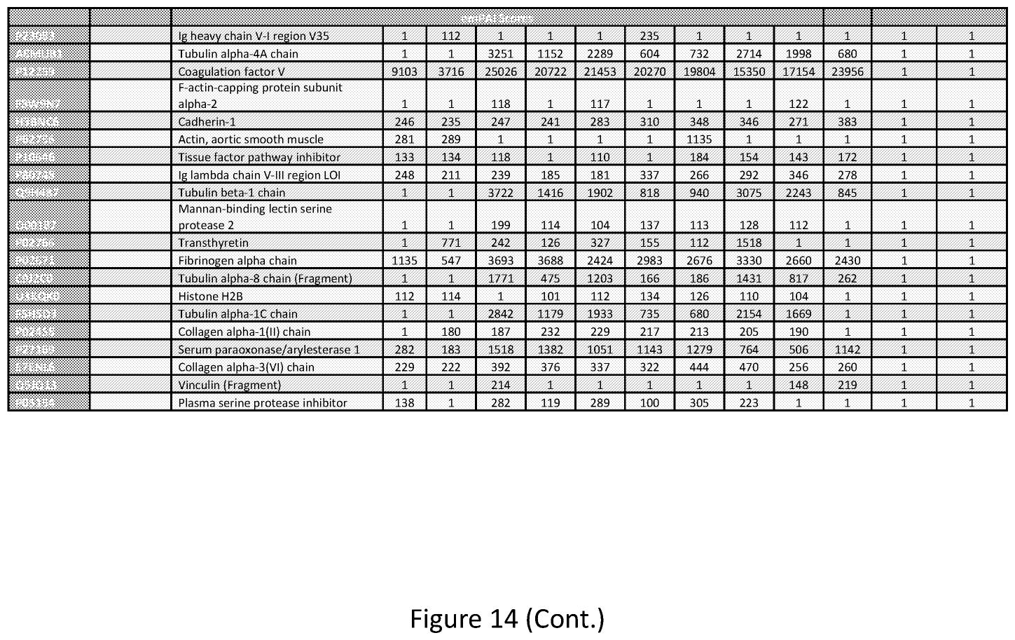

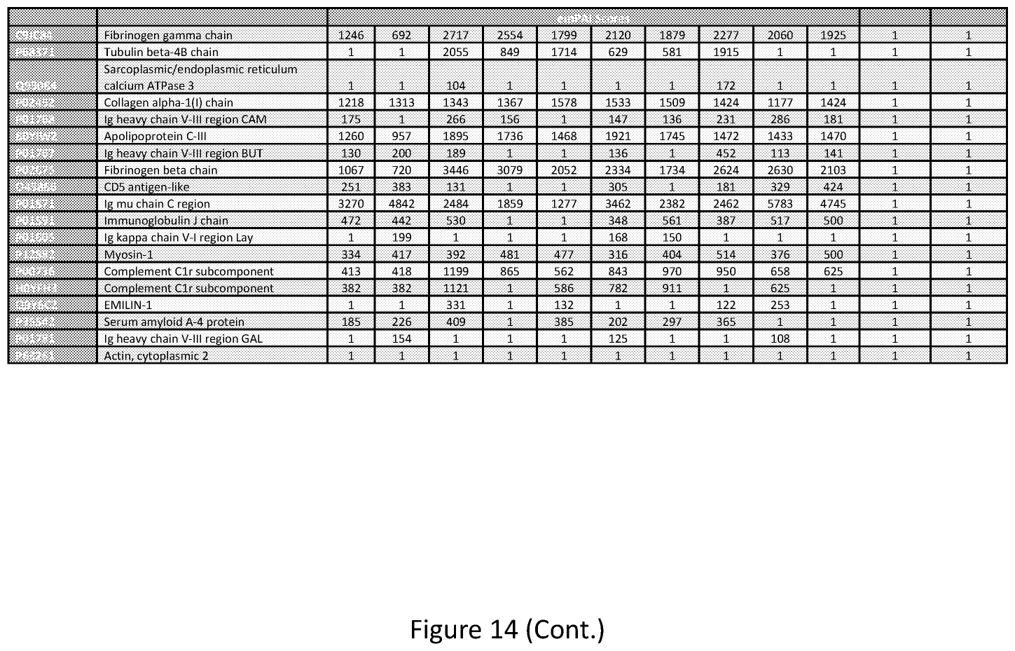

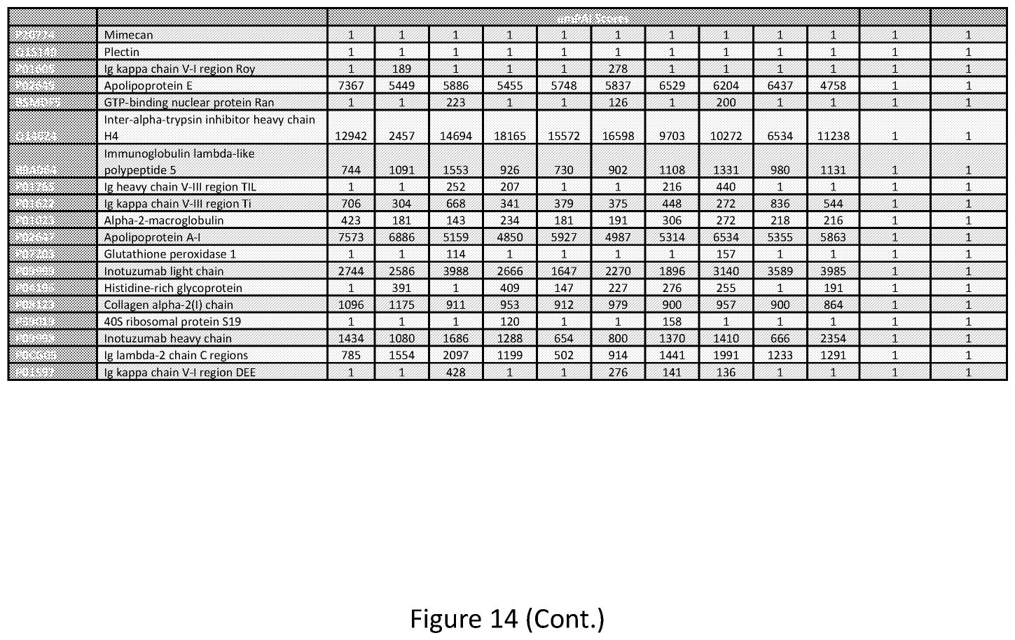

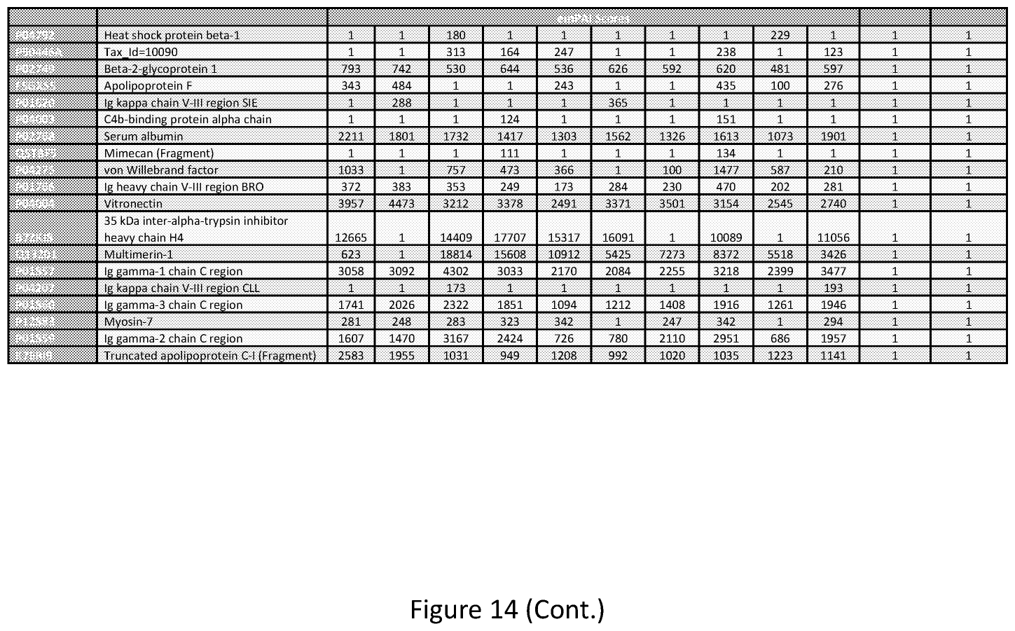

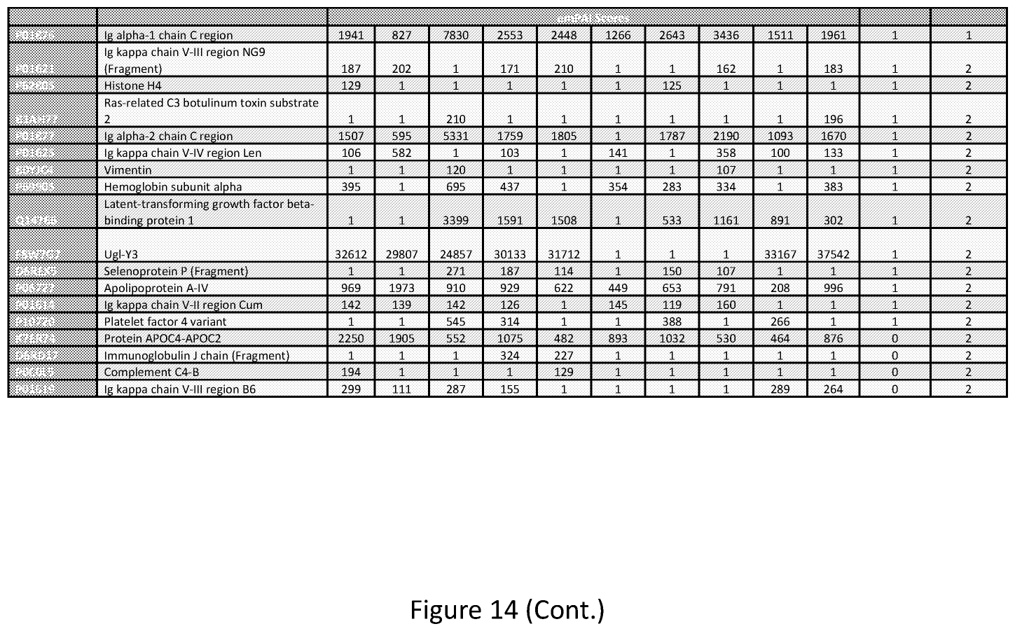

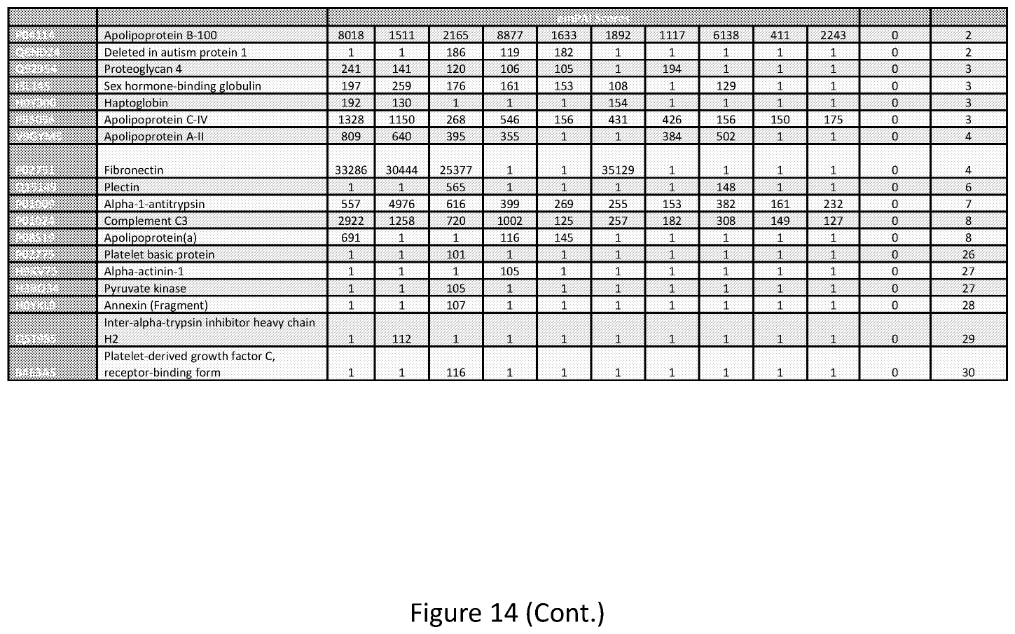

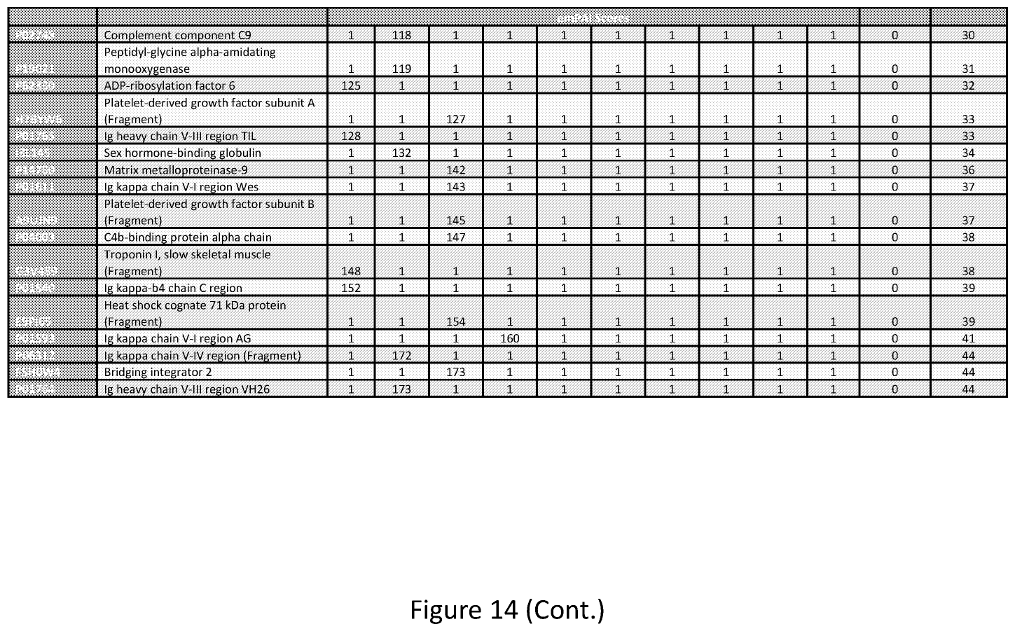

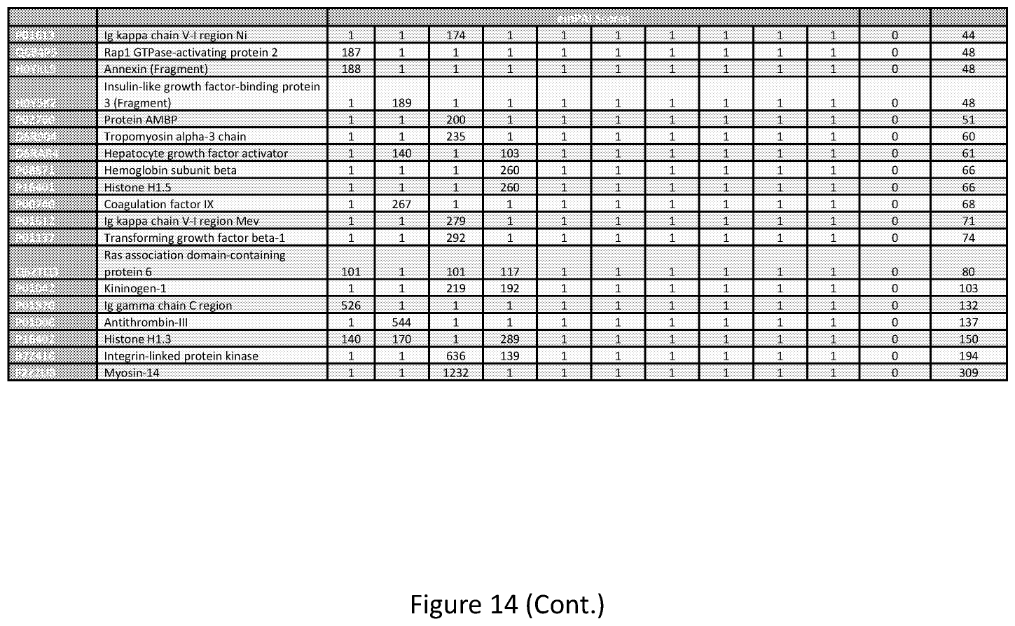

Results of MS, expressed as Exponentially Modified Protein Abundance Index (emPAI), were generated and data was sorted from highest to lowest emPAI scores. Signal to noise (S/N) ratios were calculated for average emPAI scores for ATTRwt/normal scores. The inverse S/N was also calculated. Selected markers were chosen with S/N ratios of 3 or greater (FIG. 14). Criteria for marker selection included having multiple and consistent score values in diseased, normal samples, or both categories. Missing or empty data corresponds to lower than the detectable limit. For the purpose of generating S/N values, empty data was artificially scored with a value of “1.” Eight different markers were selected from the list in Table 1 to pursue as possible markers for ATTRwt (Table 6).

TABLE 6
Selected Mass Spectrometry Hits

| | Signal to Noise emPAI Scores | | |
|---|---|---|---|
| Description | Gene Name | ATTR/Cnt | Cnt/ATTR |
| Integrin-linked protein kinase | ILK | 704 | 0 |
| Isoform 1 of four and a half LIM domains protein 1 | FHL1 | 483 | 0 |
| Transforming growth factor-beta-induced protein Ig-h3 | TGFB1 | 293 | 0 |
| Pyruvate kinase M | PKM | 254 | 0 |
| Metalloproteinase inhibitor 2 | TIMP2 | 118 | 0 |
| LIM and senescent cell antigen-like-containing domain | LIMS1 | 4 | 0 |
| protein 1 | | | |
| Serum amyloid A-1 protein | SAA1 | 3 | 0 |
| Complement C3 | C3 | 0 | 8 |

### Example 2: Patent Cohorts for Biomarker Testing

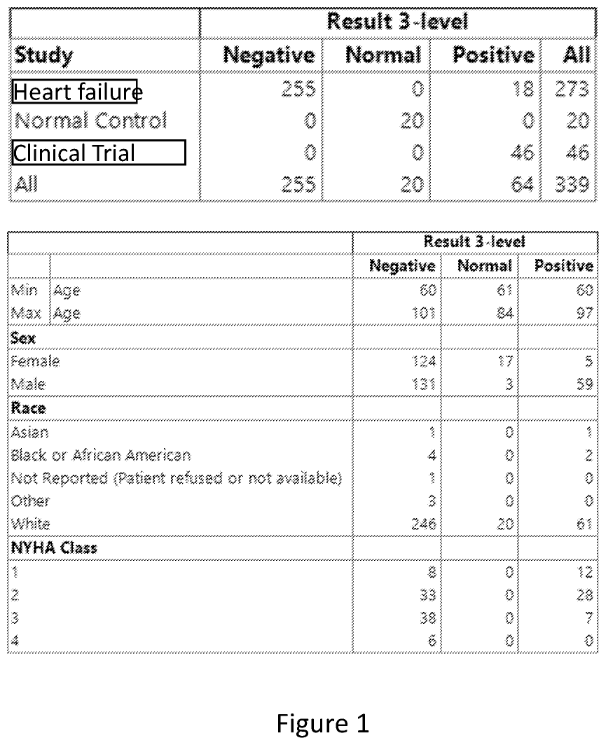

This example provides information regarding patient cohorts used in the analysis of how various ATTR Biomarkers performed in the detection of TTR-CM. As shown in FIG. 1, 273 heart failure patients, 46 clinical trial patients, and 20 independent presumed normal donors participated in this study. All individuals in this study were 60 years of age or older, and were predominantly white. The number of males and females in each subset varied. The NYHA class of the patients also varied among the subsets. Notably, only a small number of positive samples with matching negatives were available in the cohorts.

### Example 3: Summary of Test Results

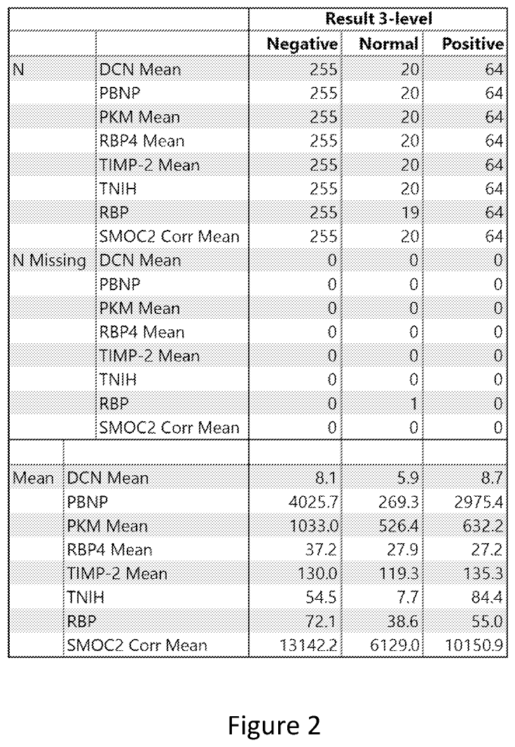

This example summarizes the data obtained from an analysis of how various ATTR Biomarkers performed in the detection of TTR-CM using the patient cohorts described in Example 2. As shown in FIG. 2, only one result was missing across all biomarkers tested. This RBP result could not be obtained due to insufficient quantity of sample for a necessary retest. In total, the in vitro diagnostic assay results were based on one test per sample; to account for higher variability of research only use assays, the mean of two test replicates were utilized.

### Example 4: Single Biomarker Classification Performance

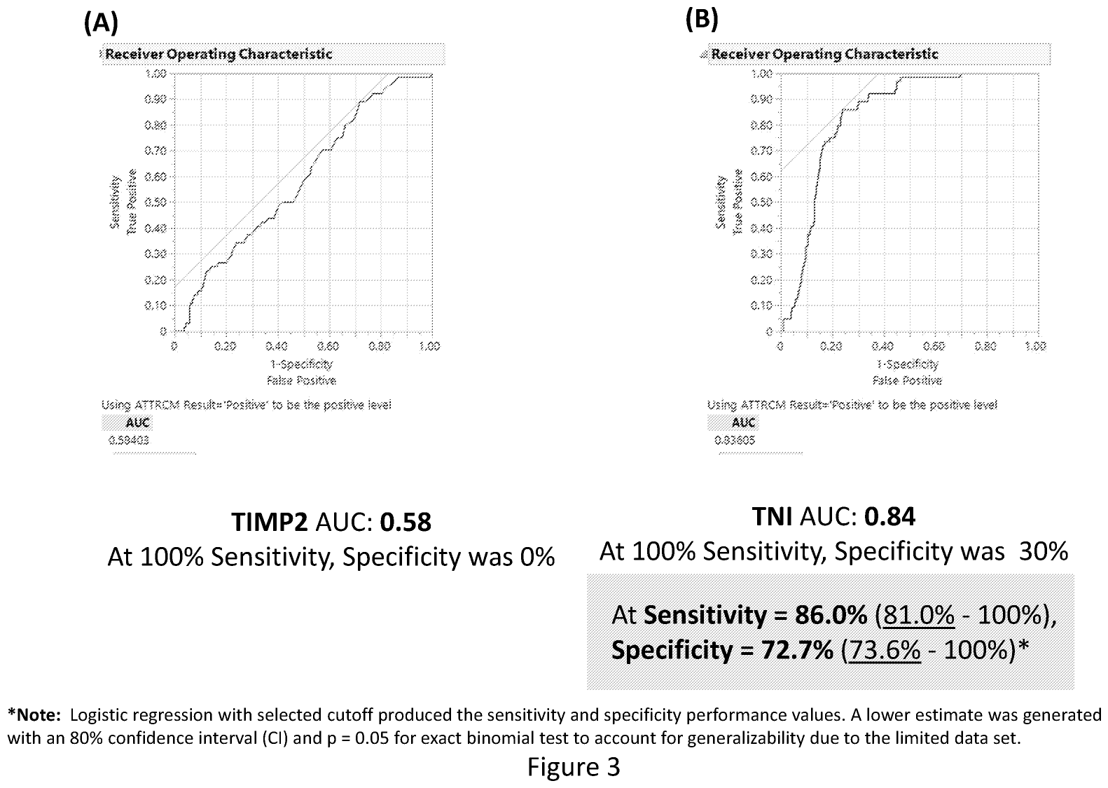

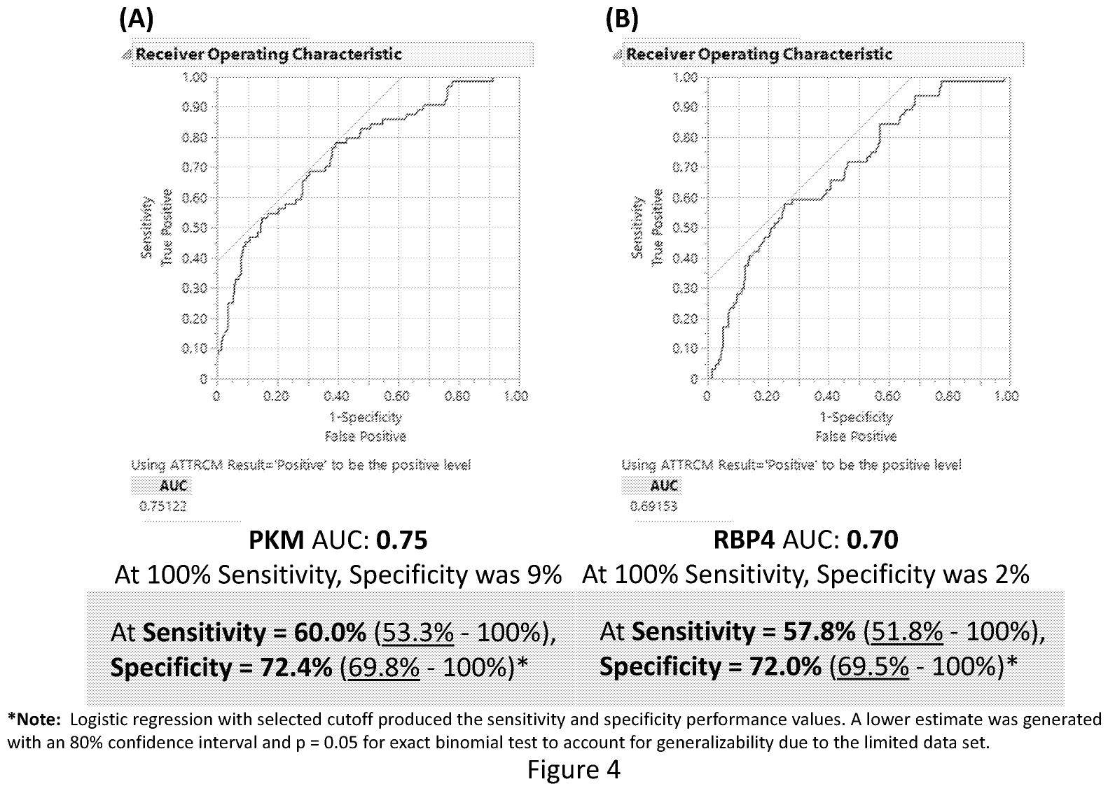

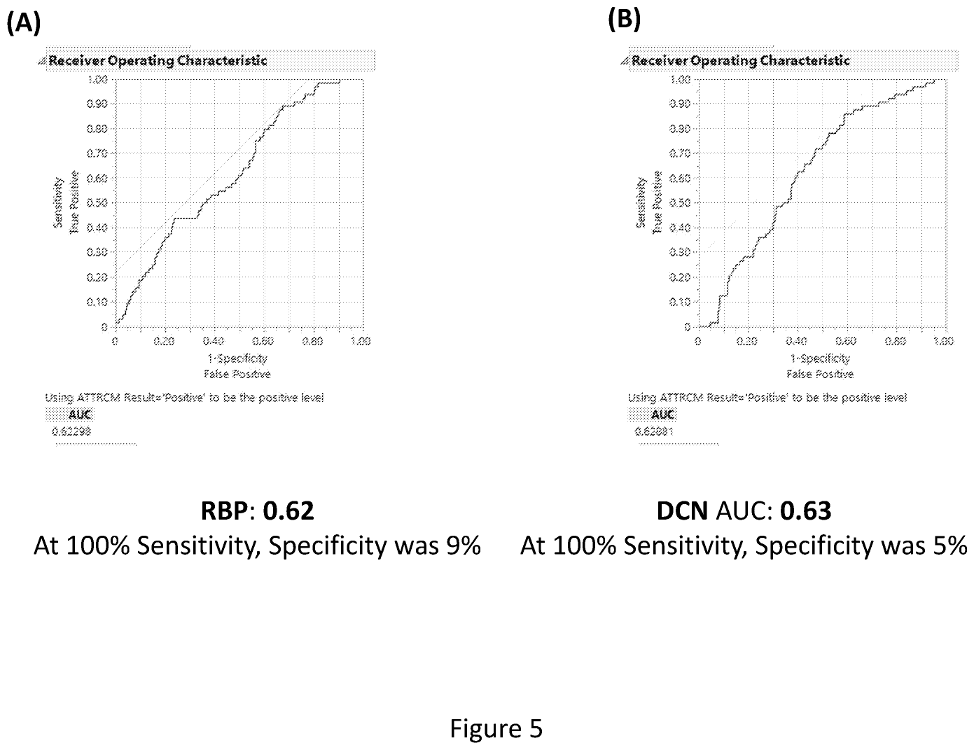

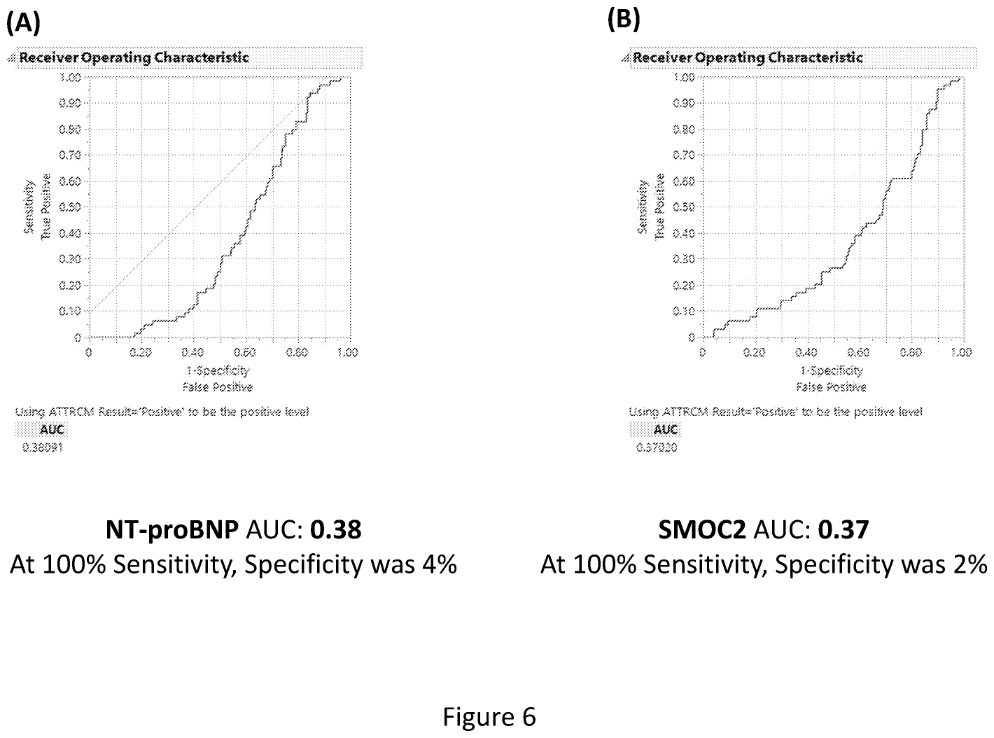

This example demonstrates an assessment of exemplary ATTR Biomarkers described herein for the ability to be used as a single biomarker for the detection of TTR-CM. The results show that certain ATTR Biomarkers described herein can perform at a sensitivity and/or specificity level sufficient that would allow them to be used alone. However, this example also demonstrates that not all ATTR Biomarkers met the criteria for both sensitivity and specificity when used as a single biomarker for assessing TTR-CM. As shown in FIGS. 3-6, TnI met both the sensitivity and specificity criteria, suggesting TnI may be useful as a single biomarker for the detection of TTR-CM. The remaining ATTR Biomarkers assessed did not meet both the sensitivity and specificity criteria set out for use a single biomarker. These biomarkers were then assessed in combination to determine whether combinations of the ATTR Biomarkers could achieve improved sensitivity and specificity when detecting for TTR-CM.

### Example 5: Single Biomarker NYHA Class Response Screening

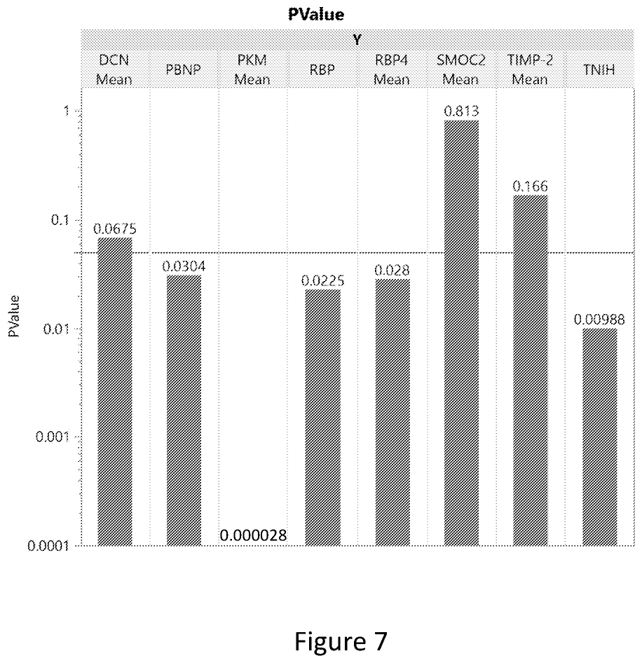

This example demonstrates an assessment of biomarker effects when considered for each NYHA class. As shown in FIG. 7, all biomarker effects for NYHA class were modeled and the p-value for the significance test was reported. The data showed that biomarker effects were statistically significant for PKM, TnI, RBP4, RBP, and NT-proBNP indicating that these ATTR Biomarkers could be useful for the detection of TTR-CM, despite the fact that not of these biomarkers met the sensitivity and specificity criteria, as discussed above in Example 4.

### Example 6: Performance Results

This example demonstrates the screening of generalized regression fits for processing the data obtained from biomarker combinations. Main effects and interactions were screened, and best fits with 4 or less biomarkers were selected for further evaluation.

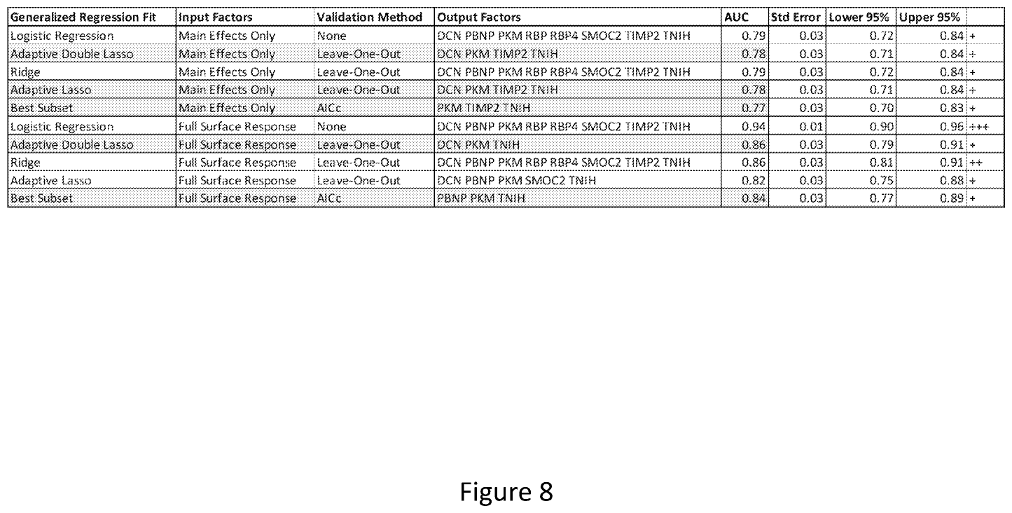

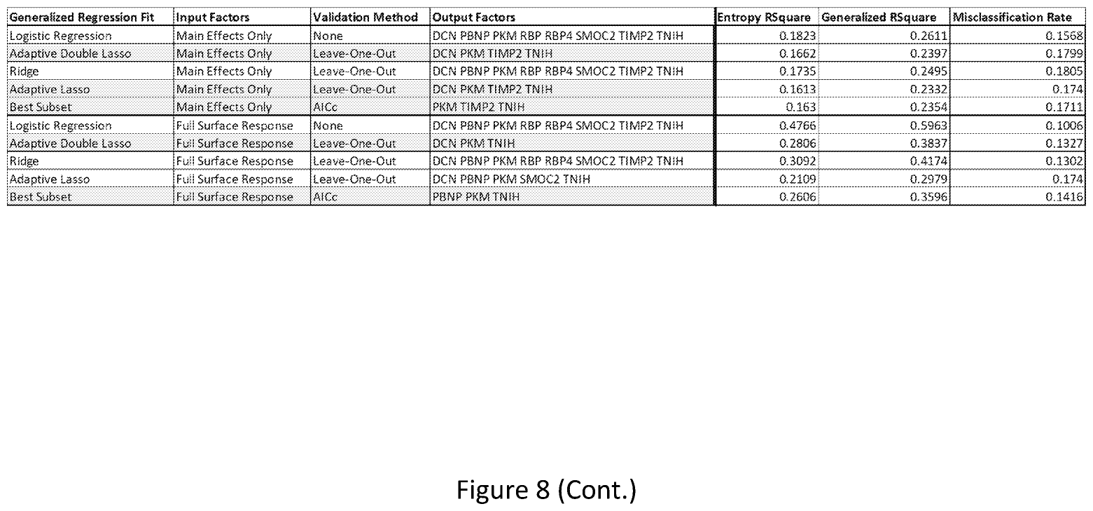

The adaptive double lasso generalized regression method with leave-one-out validation using the complete response surface as input was expected to select a near optimal model using the minimum number of effects with a small data set including some correlated biomarkers. As shown in FIG. 8, depending on the method used, different subsets of biomarkers were found informative. However, based on practical considerations, a limit of 4 biomarkers was determined. Several subsets of biomarkers and predictive models were generated. These biomarkers were further assessed using additional machine learning methods including: PKM, TnI, DCN, TIMP2, and NT-proBNP.

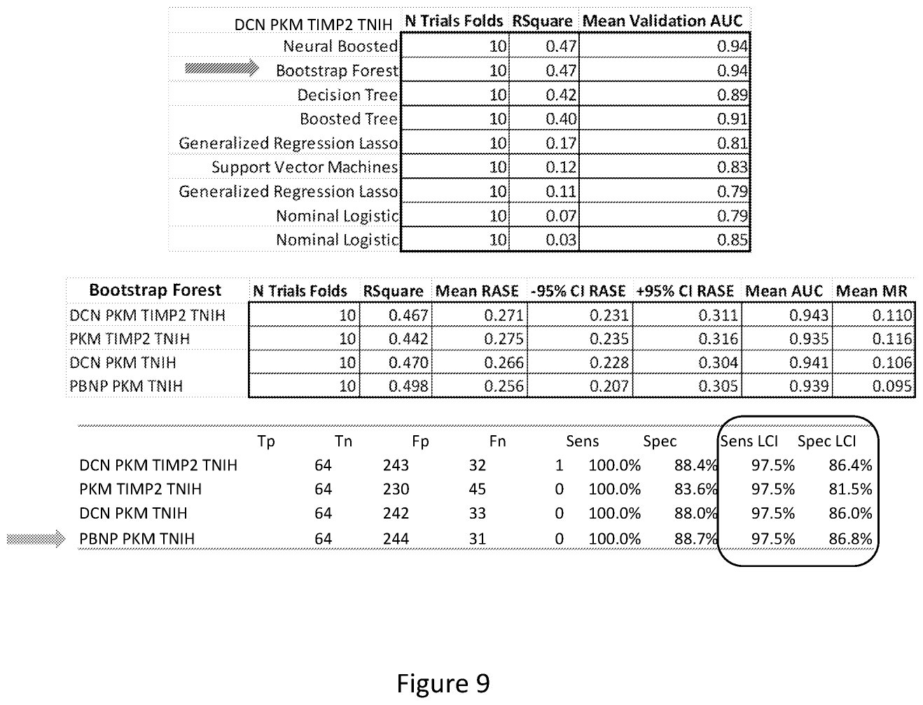

Additional model screening using combinations of PKM, TnI, DCN, TIMP2, and NT-proBNP were performed. As shown in FIG. 9, the bootstrap forest model was found to produce best results compared to other machine learning methods. The neural networks models also produced good results, but experience more false negatives.

These results indicate that three biomarker or four biomarker combinations, including TnI and PKM, in particular with NT-proBNP and/or RBP4, can detect TTR-CM with high sensitivity and specificity.

### Example 7: Identification of PKM and TIMP2 as Biomarkers Elevated in ATTR Amyloidosis Patients

This example demonstrates two identified biomarkers (PKM and TIMP2) that individually and in combination show elevated plasma levels in ATTRwt patients compared with normal human control samples.

Biomarkers selected in Table 2 were tested for immunoreactivity to normal human plasma and ATTR amyloidosis plasma by ELISA (Table 7).

TABLE 7
Biomarker Raw absorbance 450 nm values for ATTRwt and
Normal human EDTA Plasma (Part 1)

| | Raw Absorbance at 450 nm | | | | | | | |
|---|---|---|---|---|---|---|---|---|
| | FHL1 | | ILK | | TGFB1 | | LIMS-1 | |
| | CM | N | CM | N | CM | N | CM | N |
| | 0.03 | 0.09 | 0.04 | 0.04 | 2.31 | 1.70 | 0.49 | 0.30 |
| | 0.05 | 0.10 | 0.06 | 0.03 | 2.15 | 1.97 | 0.49 | 0.30 |
| | 0.03 | 0.07 | 0.09 | 0.06 | 2.14 | 2.04 | 0.51 | 0.30 |
| | 0.07 | 0.03 | 0.04 | 0.03 | 2.39 | 1.69 | 0.48 | 0.51 |
| | 0.06 | 0.04 | 0.09 | 0.06 | 1.38 | 2.39 | 0.32 | 0.41 |
| | 0.12 | 0.02 | 0.06 | 0.03 | 2.72 | 1.62 | 0.36 | 0.50 |
| | 0.02 | 0.14 | 0.03 | 0.07 | 1.69 | 1.75 | 0.29 | 0.23 |
| | 0.05 | 0.06 | 0.07 | 0.06 | 3.64 | 3.04 | 0.33 | 0.23 |
| | 0.02 | 0.07 | 0.06 | 0.05 | 1.18 | 1.66 | 0.36 | 0.25 |
| | 0.03 | 0.05 | 0.12 | 0.03 | 2.45 | 1.72 | 0.45 | 0.24 |
| | 0.02 | 0.02 | 0.06 | 0.03 | 2.19 | 2.36 | 0.34 | 0.22 |
| | 0.05 | 0.04 | 0.06 | 0.02 | 1.65 | 2.50 | 0.37 | 0.27 |
| | 0.06 | 0.03 | 0.07 | 0.03 | 1.38 | 1.96 | 0.33 | 0.30 |
| | 0.02 | 0.07 | 0.09 | 0.02 | 1.30 | 2.11 | 0.34 | 0.38 |
| | 0.05 | 0.03 | 0.04 | 0.68 | 2.41 | 1.49 | 0.40 | 0.20 |
| | 0.02 | 0.02 | 0.03 | 0.03 | 1.63 | 1.76 | 0.31 | 0.21 |
| | 0.02 | 0.08 | 0.04 | 0.08 | 2.68 | 1.44 | 0.32 | 0.27 |
| | 0.03 | 0.04 | 0.05 | 0.07 | 2.65 | 1.92 | 0.42 | 0.33 |
| | 0.03 | 0.03 | 0.05 | 0.07 | 2.47 | 1.91 | 0.46 | 0.20 |
| | 0.02 | 0.02 | 0.04 | 0.02 | 1.78 | 1.79 | 0.64 | 0.23 |
| | | | | | | | 0.67 | 0.25 |
| | | | | | | | 0.27 | 0.30 |
| | | | | | | | 0.37 | 0.25 |
| | | | | | | | 0.45 | 0.17 |
| | | | | | | | 0.32 | 0.23 |
| | | | | | | | 0.46 | 0.18 |
| | | | | | | | 0.36 | 0.26 |
| | | | | | | | 0.41 | 0.32 |
| | | | | | | | 0.44 | 0.31 |
| | | | | | | | 0.40 | 0.22 |
| Avg | 0.04 | 0.05 | 0.06 | 0.08 | 2.11 | 1.94 | 0.41 | 0.28 |

N = Normal human EDTA plasma
CM = ATTRwt human EDTA plasma

TABLE 7
Biomarker Raw absorbance 450 nm values for ATTRwt and Normal
human EDTA Plasma (Part 2)
Raw Absorbance at 450 nm

| C3 | | TIMP-2 | | PKM2 | | SAA | |
|---|---|---|---|---|---|---|---|
| CM | N | CM | N | CM | N | CM | N |
| 0.12 | 0.07 | 1.79 | 0.84 | 0.72 | 0.61 | 0.81 | 0.32 |
| 0.02 | 0.06 | 1.29 | 1.07 | 0.96 | 0.54 | 0.22 | 0.43 |
| 0.05 | 0.03 | 1.73 | 0.82 | 1.19 | 0.47 | 0.29 | 0.79 |
| 0.09 | 0.04 | 0.83 | 1.28 | 1.26 | 0.55 | 0.30 | 0.12 |
| 0.05 | 0.07 | 1.38 | 0.94 | 1.07 | 0.60 | 1.09 | 0.36 |
| 0.03 | 0.11 | 1.32 | 0.98 | 1.29 | 0.82 | 0.26 | 0.75 |
| 0.07 | 0.03 | 1.66 | 0.79 | 1.60 | 0.58 | 1.85 | 0.17 |
| 0.02 | 0.13 | 1.22 | 0.78 | 1.52 | 1.64 | 0.28 | 0.95 |
| 0.10 | 0.03 | 0.86 | 0.99 | 0.88 | 0.63 | 0.33 | 0.20 |
| 0.13 | 0.05 | 1.86 | 0.65 | 1.04 | 0.71 | 0.62 | 0.10 |
| 0.04 | 0.03 | 0.82 | 0.57 | 1.32 | 0.41 | 0.64 | 0.13 |
| 0.10 | 0.09 | 1.27 | 0.68 | 0.39 | 0.37 | 0.35 | 0.10 |
| 0.84 | 0.03 | 0.98 | 1.09 | 1.04 | 0.36 | 0.56 | 0.12 |
| 0.14 | 0.04 | 1.89 | 1.16 | 0.92 | 0.49 | 1.27 | 0.16 |
| 0.64 | 0.03 | 2.71 | 0.75 | 1.93 | 0.36 | 0.43 | 0.15 |
| 0.13 | 0.05 | 1.56 | 0.69 | 0.79 | 0.56 | 0.42 | 0.25 |
| 0.24 | 0.14 | 1.30 | 0.97 | 0.70 | 0.74 | 0.61 | 0.72 |
| 0.03 | 0.09 | 2.49 | 0.59 | 2.05 | 0.69 | 0.67 | 0.69 |
| 0.06 | 0.06 | 1.29 | 0.61 | 1.75 | 0.40 | 1.29 | 0.19 |
| 0.05 | 0.02 | 1.66 | 0.69 | 1.01 | 0.45 | 0.33 | 0.16 |
| 0.06 | 0.04 | 1.95 | 0.78 | 1.86 | 0.46 | 0.84 | 2.03 |
| 0.05 | 0.04 | 1.68 | 0.57 | 0.96 | 0.69 | 0.22 | 0.48 |
| 0.03 | 0.07 | 1.45 | 0.68 | 0.82 | 0.30 | 0.47 | 0.18 |
| 0.09 | 0.06 | 1.95 | 0.67 | 2.25 | 0.41 | 0.36 | 0.27 |
| 0.07 | 0.02 | 1.70 | 1.06 | 1.20 | 0.53 | 2.65 | 0.19 |
| 0.04 | 0.02 | 0.91 | 0.75 | 0.93 | 0.59 | 1.77 | 2.41 |
| 0.12 | 0.04 | 1.31 | 0.57 | 1.16 | 0.39 | 0.55 | 0.31 |
| 0.13 | 0.03 | 0.98 | 0.96 | 0.66 | 0.17 | 0.52 | 0.13 |
| 0.06 | 0.07 | 1.34 | 0.67 | 0.86 | 0.51 | 0.12 | 0.28 |
| 0.08 | 0.04 | 1.67 | 0.62 | 1.18 | 0.39 | 0.22 | 0.13 |
| 0.12 | 0.05 | 1.49 | 0.81 | 1.18 | 0.55 | 0.68 | 0.44 |

N = Normal human EDTA plasma
CM = ATTRwt human EDTA plasma

Signal-to-noise (S/N) ratios (calculated as raw absorbance value of the cardiomyopathy (CM) or normal (N) samples divided by the average of the normal samples for each biomarker) were determined for each biomarker assay. A cutoff value was set as the highest S/N value of the normal plasma samples for each biomarker assay, with the exception of assays for ILK and PKM, where the highest value normal sample from each was excluded for being greater than the average+(4×standard deviation).

Using S/N values and comparing these with a cutoff value for each biomarker, each sample (i.e. CM or N) was scored as positive (>cutoff) or negative (<cutoff) with 1 or 0, respectively (Table 8). Performance ratios for each biomarker, including sensitivity, specificity and accuracy, were also determined by the following calculations: % sensitivity=# of positively scored CM samples/total CM samples; % specificity=(1−# of positively scored Normal samples)×100; % accuracy=(# of reported positive CM samples+# reported negative Normal samples)/total samples (CM and N) tested×100.

TABLE 8
Scored values for CM and Normal samples and performance summary

| | | | | | | | | | TIMP2 + |
|---|---|---|---|---|---|---|---|---|---|
| | FHL1 | ILK | TGFB1 | LIMS-1 | C3 | TIMP2 | PKM2 | SAA | PKM2 |
| | Scored CM samples | | | | | | | | |
| | 0 | 0 | 0 | 0 | 0 | 1 | 0 | 0 | 1 |
| | 0 | 0 | 0 | 0 | 0 | 1 | 1 | 0 | 1 |
| | 0 | 1 | 0 | 1 | 0 | 1 | 1 | 0 | 1 |
| | 0 | 0 | 0 | 0 | 0 | 0 | 1 | 0 | 1 |
| | 0 | 1 | 0 | 0 | 0 | 1 | 1 | 0 | 1 |
| | 0 | 0 | 0 | 0 | 0 | 1 | 1 | 0 | 1 |
| | 0 | 0 | 0 | 0 | 0 | 1 | 1 | 0 | 1 |
| | 0 | 0 | 1 | 0 | 0 | 0 | 1 | 0 | 1 |
| | 0 | 0 | 0 | 0 | 0 | 0 | 1 | 0 | 1 |
| | 0 | 1 | 0 | 0 | 0 | 1 | 1 | 0 | 1 |
| | 0 | 0 | 0 | 0 | 0 | 0 | 1 | 0 | 1 |
| | 0 | 0 | 0 | 0 | 0 | 0 | 0 | 0 | 0 |
| | 0 | 0 | 0 | 0 | 1 | 0 | 1 | 0 | 1 |
| | 0 | 1 | 0 | 0 | 1 | 1 | 1 | 0 | 1 |
| | 0 | 0 | 0 | 0 | 1 | 1 | 1 | 0 | 1 |
| | 0 | 0 | 0 | 0 | 0 | 1 | 0 | 0 | 1 |
| | 0 | 0 | 0 | 0 | 1 | 1 | 0 | 0 | 1 |
| | 0 | 0 | 0 | 0 | 0 | 1 | 1 | 0 | 1 |
| | 0 | 0 | 0 | 0 | 0 | 1 | 1 | 0 | 1 |
| | 0 | 0 | 0 | 1 | 0 | 1 | 1 | 0 | 1 |
| | | | | 1 | 0 | 1 | 1 | 0 | 1 |
| | | | | 0 | 0 | 1 | 1 | 0 | 1 |
| | | | | 0 | 0 | 1 | 1 | 0 | 1 |
| | | | | 0 | 0 | 1 | 1 | 0 | 1 |
| | | | | 0 | 0 | 1 | 1 | 1 | 1 |
| | | | | 0 | 0 | 0 | 1 | 0 | 1 |
| | | | | 0 | 0 | 1 | 1 | 0 | 1 |
| | Scored Normal samples | | | | | | | | |
| | 0 | 0 | 0 | 0 | 0 | 0 | 0 | 0 | 0 |
| | 0 | 0 | 0 | 0 | 0 | 0 | 0 | 0 | 0 |
| | 0 | 0 | 0 | 0 | 0 | 0 | 0 | 0 | 0 |
| | 0 | 0 | 0 | 0 | 0 | 0 | 0 | 0 | 0 |
| | 0 | 0 | 0 | 0 | 0 | 0 | 0 | 0 | 0 |
| | 0 | 0 | 0 | 0 | 0 | 0 | 0 | 0 | 0 |
| | 0 | 0 | 0 | 0 | 0 | 0 | 0 | 0 | 0 |
| | 0 | 0 | 0 | 0 | 0 | 0 | 1 | 0 | 1 |
| | 0 | 0 | 0 | 0 | 0 | 0 | 0 | 0 | 0 |
| | 0 | 0 | 0 | 0 | 0 | 0 | 0 | 0 | 0 |
| | 0 | 0 | 0 | 0 | 0 | 0 | 0 | 0 | 0 |
| | 0 | 0 | 0 | 0 | 0 | 0 | 0 | 0 | 0 |
| | 0 | 0 | 0 | 0 | 0 | 0 | 0 | 0 | 0 |
| | 0 | 0 | 0 | 0 | 0 | 0 | 0 | 0 | 0 |
| | 0 | 1 | 0 | 0 | 0 | 0 | 0 | 0 | 0 |
| | 0 | 0 | 0 | 0 | 0 | 0 | 0 | 0 | 0 |
| | 0 | 0 | 0 | 0 | 0 | 0 | 0 | 0 | 0 |
| | 0 | 0 | 0 | 0 | 0 | 0 | 0 | 0 | 0 |
| | 0 | 0 | 0 | 0 | 0 | 0 | 0 | 0 | 0 |
| | 0 | 0 | 0 | 0 | 0 | 0 | 0 | 0 | 0 |
| | | | | 0 | 0 | 0 | 0 | 0 | 0 |
| | | | | 0 | 0 | 0 | 0 | 0 | 0 |
| | | | | 0 | 0 | 0 | 0 | 0 | 0 |
| | | | | 0 | 0 | 0 | 0 | 0 | 0 |
| | | | | 0 | 0 | 0 | 0 | 0 | 0 |
| | | | | 0 | 0 | 0 | 0 | 0 | 0 |
| | | | | 0 | 0 | 0 | 0 | 0 | 0 |
| | | | | 0 | 0 | 0 | 0 | 0 | 0 |
| | | | | 0 | 0 | 0 | 0 | 0 | 0 |
| | | | | 0 | 0 | 0 | 0 | 0 | 0 |
| Reported Positive | 0 | 4 | 1 | 3 | 4 | 22 | 25 | 1 | 28 |
| Reported | 20 | 19 | 20 | 30 | 30 | 30 | 29 | 30 | 29 |
| Negative | | | | | | | | | |
| Sensitivity | 0% | 20% | 5% | 10% | 13% | 73% | 83% | 3% | 93% |
| Specificity | 100% | 95% | 100% | 100% | 100% | 100% | 97% | 100% | 97% |
| Accuracy | 50% | 58% | 53% | 55% | 57% | 87% | 90% | 52% | 95% |

Performance scores for various biomarkers tested showed high sensitivity, specificity, and accuracy for two individual markers, TIMP2 (72% sensitivity, 100% specificity, 87% accuracy) and PKM (83% sensitivity, 97% specificity, 90% accuracy). When the two markers, TIMP2 and PKM are combined across the same set of samples, sensitivity, specificity, and accuracy significantly improves (93% sensitivity, 97% specificity, 95% accuracy), suggesting that TIMP2 and PKM combined can serve as sensitive and specific markers for the identification of ATTRwt plasma.

### Example 8: Calculation of Biomarker Threshold Values

This example demonstrates identification of threshold values for biomarkers of ATTR amyloidosis.

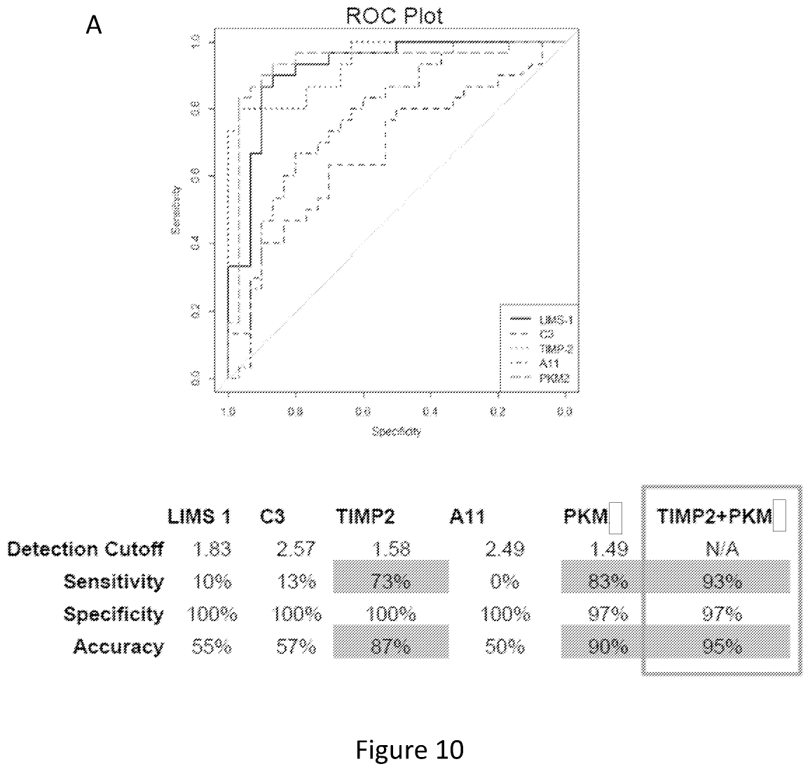

A total of N=30 diagnosed TTR-CM patient plasma samples and N=30 normal donor samples were assessed for expression of PKM, TIMP2, LIMS1, C3 and A11. Sensitivity, specificity, and accuracy were calculated as described above. Sensitivity and specificity calculations assumed no undiagnosed TTR-CM donors. Optimized detection cutoffs for each assay were determined by maximizing sensitivity while retaining approximately 100% specificity (Table 9 and FIG. 10).

The disclosed methods may be performed manually or may be automated. For example, the disclosed methods may use ADVIA CENTAUR® immunoassay system or ATELLICA @™. For example, a system may be automated to perform the following actions:
1. the samples were dispensed into cuvettes.
2. The buffer containing the solid support with anti-human IgM antibody bound thereto is dispensed and incubated, for example, at 37° C. for 18.25 minutes.
3. The solid support is detached/separated, the cuvette is aspirated and washed with washing reagent.
4. The buffer containing the chemiluminescent-tagged NS1 antigen is dispensed and incubated, for example, at 37° C. for 18 minutes.
5. The solid support is detached/separated, the cuvette is aspirated and washed with washing reagent.
6. Dispensing a chemiluminescent reagent to initiate a chemiluminescent reaction.
7. Reporting the result according to the option selected by the user.

In a particular embodiment, the disclosed immunoassays may be suitable for use on the ADVIA CENTAUR immunoassay system (Siemens Healthcare, AG), and/or the ADVIA CENTAUR immunoassay system may be automated to perform the actions described above.

TABLE 9
ROC curve data for optimized detection cutoffs of biomarkers

| LIMS | | | C3 | | | TIMP2 | | | A11 | | |
|---|---|---|---|---|---|---|---|---|---|---|---|
| Cutoff | | | Cutoff | | | Cutoff | | | Cutoff | | |
| Value | Specificity | Sensitivity | Value | Specificity | Sensitivity | Value | Specificity | Sensitivity | Value | Specificity | Sensitivity |
| 0.60 | 0% | 100% | 0.30 | 0% | 100% | 0.70 | 0% | 100% | 0.40 | 0% | 100% |
| 0.60 | 7% | 100% | 0.30 | 7% | 100% | 0.70 | 13% | 100% | 0.40 | 7% | 100% |
| 0.60 | 13% | 100% | 0.30 | 23% | 87% | 0.70 | 20% | 100% | 0.40 | 17% | 100% |
| 0.60 | 20% | 100% | 0.30 | 33% | 83% | 0.70 | 33% | 100% | 0.40 | 23% | 100% |
| 0.60 | 37% | 100% | 0.30 | 50% | 77% | 0.70 | 43% | 100% | 0.40 | 37% | 97% |
| 0.60 | 53% | 100% | 0.30 | 53% | 73% | 0.70 | 47% | 100% | 0.40 | 53% | 87% |
| 0.60 | 60% | 97% | 0.30 | 57% | 63% | 0.70 | 57% | 100% | 0.40 | 63% | 77% |
| 0.60 | 60% | 93% | 0.30 | 70% | 60% | 0.70 | 63% | 97% | 0.40 | 73% | 67% |
| 0.60 | 80% | 90% | 0.30 | 73% | 50% | 0.70 | 67% | 90% | 0.40 | 87% | 50% |
| 0.60 | 87% | 73% | 0.30 | 80% | 47% | 0.70 | 67% | 90% | 0.40 | 90% | 43% |
| 0.60 | 87% | 67% | 0.30 | 83% | 47% | 0.70 | 67% | 87% | 0.40 | 90% | 43% |
| 0.60 | 87% | 50% | 0.30 | 83% | 43% | 0.70 | 73% | 83% | 0.40 | 90% | 37% |
| 0.60 | 90% | 50% | 0.30 | 87% | 40% | 0.70 | 83% | 80% | 0.40 | 90% | 30% |
| 0.60 | 90% | 43% | 0.30 | 90% | 37% | 0.70 | 83% | 80% | 0.40 | 93% | 27% |
| 0.60 | 93% | 37% | 0.30 | 90% | 30% | 0.70 | 90% | 80% | 0.40 | 93% | 23% |
| 0.60 | 93% | 37% | 0.30 | 90% | 30% | 0.70 | 93% | 80% | 0.40 | 93% | 17% |
| 0.60 | 93% | 23% | 0.30 | 93% | 30% | 0.70 | 93% | 80% | 0.40 | 93% | 13% |
| 0.60 | 93% | 17% | 0.30 | 93% | 30% | 0.70 | 97% | 80% | 0.40 | 93% | 3% |
| 0.60 | 93% | 10% | 0.30 | 93% | 17% | 0.70 | 97% | 77% | 0.40 | 97% | 3% |
| 0.60 | 100% | 7% | 0.30 | 97% | 13% | 0.70 | 97% | 77% | 0.40 | 97% | 3% |
| 1.90 | 100% | 7% | 2.60 | 100% | 13% | 1.60 | 100% | 67% | 2.50 | 100% | 0% |
| 0.07 | | | 0.12 | | | 0.05 | | | 0.11 | | |

### Example 9: Patient Assessment

A patient at risk of TTR-CM is tested for a level of each ATTR Biomarker in a combinations of ATTR Biomarkers, as disclosed herein, e.g., in Tables 2-5. A blood sample is taken from the patient and levels are measured using a Siemens Atellica® system or the Siemens Advia Centaur® system to obtain an ATTR Biomarker profile for the patient. As needed, the patient's demographic factors and image-based biomarkers are considered. The patients ATTR Biomarker profile is used to calculate an ATTR Biomarker score. Based on the ATTR Biomarker score, patient is categorized as having TTR-CM. Administration of a TTR stabilizer or other cardiac medication is recommended.

### Example 10: Patient Assessment

A patient at risk of TTR-CM is tested for a level of each ATTR Biomarker in a combinations of ATTR Biomarkers, as disclosed herein, e.g., in Tables 2-5. A blood sample is taken from the patient and levels are measured using a Siemens Atellica® system or the Siemens Advia Centaur® system to obtain an ATTR Biomarker profile for the patient. As needed, the patient's demographic factors and image-based biomarkers are considered. The patient's ATTR Biomarker profile is compared to a reference ATTR Biomarker profile. Based on the comparison, patient is categorized as having TTR-CM. Administration of a TTR stabilizer or other cardiac medication is recommended.

### Example 11: Patient Assessment

A patient at risk of TTR-CM is tested for a level of each ATTR Biomarker in a combinations of ATTR Biomarkers, as disclosed herein, e.g., in Tables 2-5. A blood sample is taken from the patient and levels are measured using a Siemens Atellica® system or the Siemens Advia Centaur® system to obtain an ATTR Biomarker profile for the patient. As needed, the patient's demographic factors and image-based biomarkers are considered. The patient's ATTR Biomarker profile is used to calculate an ATTR Biomarker score. Based on the ATTR Biomarker score, patient is categorized as being at risk of having TTR-CM. Further cardiomyopathy testing is recommended.

### Example 12: Patient Assessment

A patient at risk of TTR-CM is tested for a level of each ATTR Biomarker in a combinations of ATTR Biomarkers, as disclosed herein, e.g., in Tables 2-5. A blood sample is taken from the patient and levels are measured using a Siemens Atellica® system or the Siemens Advia Centaur® system to obtain an ATTR Biomarker profile for the patient. As needed, the patient's demographic factors and image-based biomarkers are considered. The patient's ATTR Biomarker profile is compared to a reference ATTR Biomarker profile. Based on the comparison, patient is categorized as being at risk of having TTR-CM. Further cardiomyopathy testing is recommended.

### Example 13: Population Assessment

1000 patients at risk of TTR-CM are tested for a level of each ATTR Biomarker in a combinations of ATTR Biomarkers, as disclosed herein, e.g., in Tables 2-5. A blood sample is taken from the patient and levels are measured using a Siemens Atellica® system or the Siemens Advia Centaur® system to obtain an ATTR Biomarker profile for the patient. As needed, the patient's demographic factors and image-based biomarkers are considered. The ATTR Biomarker profiles of the patients are used to calculate respective ATTR Biomarker scores. A subset of patients is categorized as being at risk of having TTR-CM. Further cardiomyopathy testing is recommended.

### Example 14: Clinical Utility of Noninvasive, Accurate TTR-CM

This Example demonstrates the benefit to the public of offering a noninvasive TTR-CM assay that is both sensitive and specific, and is easily complied with. This example demonstrates that the reluctance to undergo evasive cardiomyopathy testing is common, and that it can have severe health consequences if TTR-CM is not detected early.

A patient demonstrated symptoms of TTR-CM but refused a heart biopsy. The patient's primary care physician ordered an assessment of the patient's level of each ATTR Biomarker in a combinations of ATTR Biomarkers, as disclosed herein, e.g., in Tables 2-5. The results indicated that the patient was at a high risk for TTR-CM. The patient consulted with their physician and was convinced to schedule further cardiomyopathy testing, which reveal evidence of TTR-CM.

### REFERENCES

1. Gertz, M. A., Benson, M. D., Dyck, P. J., Grogan, M. Coelho, T., Crus, M., Berk, J. L., Plante-Bordeneuve, V., Schmidt, H. H., Merlini, G. Diagnosis, Prognosis, and Therapy of Transthyretin Amyloidosis. JACC 66:2452-2466 (2015).
2. Ashley 2004
3. Krishnamurthy, R., Cheong, B., Muthypillai, R. Tools for cardiovascular magnetic resonance imaging. Current Cardiology Reviews, 9:185-190, 2013.
4. Doltra, A., Amundsen, B. H., Gebker, R., Fleck, E., Kelle, S. Emerging concepts for myocardial late gadolinium enhancement MRI. Curr Cardiol Rev. 9(3):185-90, 2013.
5. Bokhari, S., Castano, A., Pozniakoff, T., Deslisle, S., Latif, F., Maurer, M. S., (99m)Tc-pyrophosphate scintigraphy for differentiating light-chain cardiac amyloidosis from the transthyretin-related familial and senile cardiac amyloidoses. Circ Cardiovasc Imaging. 6(2):195-201, 2013.
6. Crotty, T. B. Li, C. Y., Edwards, W. D., Suman, V. J. Amyloidosis and endomyocardial biopsy: Correlation of extent and pattern of deposition with amyloid immunophenotype in 100 cases. Cardiovascular Pathology 4:39-42, 1995.
7. Arvanitis, M. et al. Identification of Transthyretin Cardiac Amyloidosis Using Serum Retinol-Binding Protein 4 and a Clinical Prediction Model. JAMA Cardiol., 2017.

All publications and patents cited in this disclosure (including those listed above) are herein incorporated by reference as if each individual publication or patent were specifically and individually indicated to be incorporated by reference.

### EQUIVALENTS

Those skilled in the art will recognize, or be able to ascertain using no more than routine experimentation, many equivalents to the specific embodiments of the disclosure described herein. The scope of the present disclosure is not intended to be limited to the above Description, but rather is as set forth in the following claims:

1. A method comprising:
detecting a level of each of two or more transthyretin amyloidosis (ATTR) Biomarkers in a sample, wherein the two or more ATTR Biomarkers comprise:
(i) troponin I (TnI),
(ii) pyruvate kinase muscle isoform 1 (PKM1),
(iii) pyruvate kinase muscle isoform 2 (PKM2),
(iv) N-terminal-pro hormone B-type natriuretic peptide (NT-proBNP),
(v) retinol binding protein 4 (RBP4),
(vi) tissue inhibitor of metalloproteinase 2 (TIMP2),
(vii) neurofilament light chain (NfL), or
(viii) a combination thereof.

2. A method comprising:
detecting a level of each of two or more ATTR Biomarkers in a sample, wherein the two or more ATTR Biomarkers comprise (i) TnI and PKM1, (ii) TnI and PKM2, or (iii) TnI, PKM1, and PKM2.

3. A method comprising:
(a) detecting a level of each of two or more ATTR Biomarkers in a sample obtained from a subject to obtain an ATTR Biomarker profile; and
(b) using the ATTR Biomarker profile to compute a ATTR Biomarker score, wherein the two or more ATTR Biomarkers comprise:
(i) troponin I (TnI),
(ii) pyruvate kinase muscle isoform 1 (PKM1),
(iii) pyruvate kinase muscle isoform 2 (PKM2),
(iv) N-terminal-pro hormone B-type natriuretic peptide (NT-proBNP),
(v) retinol binding protein 4 (RBP4),
(vi) tissue inhibitor of metalloproteinase 2 (TIMP2),
(vii) neurofilament light chain (NfL), or
(viii) a combination thereof, and
wherein using the ATTR Biomarker profile to compute an ATTR Biomarker score comprises applying an algorithm to the ATTR Biomarker profile to compute an ATTR Biomarker score, wherein the algorithm is derived from a decision tree methodology, a neural boosted methodology, a bootstrap forest methodology, a boosted tree methodology, or a support vector machines methodology.

4. The method of claim 3, wherein the subject is a human subject.

5. The method of claim 1, wherein the sample comprises blood, serum, plasma, or cardiac tissue.

6. The method of claim 3, further comprising using the ATTR Biomarker score to determine if a subject from which the sample was obtained is at risk of or suffering from transthyretin amyloid cardiomyopathy (TTR-CM).

7. The method of claim 3, further comprising using the ATTR Biomarker score to diagnose a subject with TTR-CM.

8. The method of claim 3, further comprising using the ATTR Biomarker score to determine if a subject from which the sample was obtained is selected for one or more cardiomyopathy tests.

9. The method of claim 3, further comprising using the ATTR Biomarker score to determine if a subject from which the sample was obtained is selected to receive one or more doses of a TTR stabilizing agent.

10. The method of claim 1, wherein the two or more ATTR Biomarkers do not comprise PKM1.

11. The method of claim 1, wherein the two or more ATTR Biomarkers do not comprise PKM2.

12. The method of claim 1, wherein the two or more ATTR Biomarkers do not comprise either PKM1 or PKM2.

13. The method of claim 1, wherein the two or more ATTR Biomarkers do not comprise SMOC-2.

14. The method of claim 1, wherein the two or more ATTR Biomarkers do not comprise DCN.

15. The method of claim 1, wherein the two or more ATTR Biomarkers do not comprise either SMOC-2 or DCN.

16. A non-transitory computer readable medium containing executable instructions that when executed cause a processor to perform operations comprising the method of claim 1.

17. A composition comprising:
(a) one or more ATTR Biomarkers, wherein the one or more ATTR Biomarkers comprise:
(i) TnI,
(ii) PKM1,
(iii) PKM2,
(iv) NT-proBNP,
(v) RBP4,
(vi) TIMP2,
(vii) NfL, or
(viii) a combination thereof; and
(b) one or more anti-ATTR Biomarker agents, wherein the one or more anti-ATTR Biomarker agents comprise:
(i) an anti-TnI agent,
(ii) an anti-PKM1 agent,
(iii) an anti-PKM2 agent,
(iv) an anti-NT-proBNP agent,
(v) an anti-RBP4 agent,
(vi) an anti-TIMP2 agent,
(vii) an anti-NfL agent, or
(viii) a combination thereof.

18. A kit for detecting TTR-CM, said kit comprising:
(a) one or more anti-ATTR Biomarker agents, wherein the one or more anti-ATTR Biomarker agents comprise:
(i) an anti-TnI agent,
(ii) an anti-PKM1 agent,
(iii) an anti-PKM2 agent,
(iv) an anti-NT-proBNP agent,
(v) an anti-RBP4 agent,
(vi) an anti-TIMP2 agent,
(vii) an anti-NfL agent, or
(viii) a combination thereof; and
(b) instructions for use.

19. A kit for detecting TTR-CM, said kit comprising:
(a) one or more anti-ATTR Biomarker agents, wherein the one or more anti-ATTR Biomarker agents comprise (i) an anti-TnI agent and an anti-PKM1 agent, (ii) an anti-TnI agent and an anti-PKM2 agent, or (iii) an anti-TnI agent, an anti-PKM1 agent, and an anti-PKM2 agent; and
(b) instructions for use.

20. A kit comprising:
(a) one or more anti-ATTR Biomarker agents, wherein the one or more anti-ATTR Biomarker agents comprise an anti-NT-proBNP agent, an anti-TnI agent, and an anti-RBP4 agent; and
(b) instructions for use.

21. The kit of claim 18, wherein the one or more anti-ATTR Biomarker agents comprise one or more antibody agents.

22. The kit of claim 21, wherein one or more of the antibody agents are labeled with a detectable moiety.

23. The kit of claim 18, further comprising one or more control samples.

24. The kit of claim 23, wherein the control samples comprise one or more ATTR Biomarker standards.

25. Use of a kit according to claim 18 in an in vitro diagnostic assay to diagnose TTR-CM in a subject.
